(19) United States
(12) Patent Application Publication
(10) Pub. No.: US 2026/0041647 A1
(43) Pub. Date: Feb. 12, 2026

(54) PRECLINICAL DEVELOPMENT OF A ROMIDEPSIN NANOPARTICLE DEMONSTRATES SUPERIOR TOLERABILITY AND EFFICACY IN MODELS OF HUMAN T-CELL LYMPHOMA AND LARGE GRANULAR LYMPHOCYTE LEUKEMIA

(71) Applicant: University of Virginia Patent Foundation, Charlottesville, VA (US)

(72) Inventors: Owen O'Connor, Scarsdale, NY (US); Ipsita Pal, Charlottesville, VA (US); Anuradha Illendula, Crozet, VA (US)

(73) Assignee: University of Virginia Patent Foundation, Charlottesville, VA (US)

(21) Appl. No.: 19/268,929

(22) Filed: Jul. 14, 2025

Related U.S. Application Data
(60) Provisional application No. 63/670,581, filed on Jul. 12, 2024.

(51) Int. Cl. A61K 9/51; A61K 38/15; A61K 45/06; A61K 47/10; A61K 49/00; A61P 35/02

(52) U.S. Cl. CPC A61K 9/5153; A61K 38/15; A61K 45/06; A61K 47/10; A61K 49/0032; A61K 49/0056; A61P 35/02

(57) ABSTRACT

Provided are compositions that include histone deacetylase (HDAC) inhibitors encapsulated in and/or otherwise associated with detectable nanoparticles, and methods for using the same in medical and veterinary applications including but not limited to treating diseases, disorders, and/or conditions associated with sensitivity to HDAC inhibitors; inhibiting the growth, proliferation, and/or metastasis of a tumor and/or a cancer associated with sensitivity to HDAC inhibitors, and for treating inflammatory and/or an autoimmune diseases, disorders, and/or conditions associated with sensitivity to HDAC inhibitors. Also provided are methods for imaging cells, tissues, organs, and/or other targets in subject.

### CROSS REFERENCE TO RELATED APPLICATION

This application claims benefit of U.S. Provisional Application Ser. No. 63/670,581 filed Jul. 12, 2024, the disclosure of which is incorporated herein by reference in its entirety.

### TECHNICAL FIELD

The presently disclosed subject matter relates in some embodiments to methods for treating diseases, disorders, and conditions with histone deacetylase inhibitors (HDACi), particularly romidepsin. In some embodiments, the disease, disorder, or condition is a tumor and/or a cancer.

### BACKGROUND

The histone deacetylase inhibitors (HDACi) are widely recognized as an important class of drugs for the treatment of T-cell malignancies. To date, four HDACi have been approved globally for the treatment of patients with relapsed or refractory (R/R) cutaneous (CTCL) and peripheral T-cell lymphomas (PTCL). Vorinostat, a hydroxamic acid derivative, was the first HDACi to be approved for the treatment of any disease, being approved in 2006 for the treatment of R/R CTCL (O'Connor et al., 2006; Olsen et al., 2007). Romidepsin is a bicyclic peptide originally approved for patients with R/R CTCL and PTCL in 2011 and 2014 respectively (Piekarz et al., 2009; Whittaker et al., 2010; Coiffier et al., 2012). Belinostat and tucidinostat (formerly known as chidamide), both hydroxamic acid derivates, were approved for R/R PTCL in 2014 (O'Connor et al., 2015), with tucidinostat being approved only by the National Medical Products Administration (NMPA, formerly the CFDA) in China (Shi et al., 2015). Tucidinostat has also received regulatory approval in China in combination with an aromatase inhibitor for post-menopausal women previously treated with an endocrine therapy who have hormone positive, HER2 negative recurrent or locally progressive advanced metastatic breast cancer (Jiang et al., 2019). Panobinostat, a very potent hydroxamic acid HDACi with early promising experiences in T-cell neoplasms (Prince et al., 2009), was approved in 2015 in combination with bortezomib for patients with R/R multiple myeloma. Panobinostat was subsequently withdrawn in 2021 after the sponsor concluded the Phase IV commitment was not feasible. Across patients with R/R PTCL, recognizing that there are no randomized studies of one HDACi against another, these drugs produce overall response rates of approximately 25% with a progression free survival of 3-4 months with a duration of response of approximately one year (Piekarz et al., 2009; Coiffier et al., 2012; O'Connor et al., 2015; Shi et al., 2015). While these drugs appear to benefit patients across the nearly 36 different subtypes of PTCL, there is some retrospective data to suggest that patients with PTCL of a T-follicular helper phenotype (PTCL-TFH) and angioimmunoblastic T-cell lymphoma (AITL) may have slightly greater benefit from an HDACi, with some studies noting the described aberrations in epigenetic genes as one possible explanation for the increased sensitivity (Tao et al., 2023).

While there is little dispute that the HDACi can produce meaningful cytotoxicity across a variety of cancer cell lines, clinically the drugs appear to have a unique therapeutic benefit in patients with T-cell lymphomas, though there are no compelling explanations for this disease specific activity. Histone deacetylases catalyze the deacetylation of histone and non-histone proteins. Deacetylation of histone leads to the condensation of chromatin (to heterochromatin) and transcriptional repression (Saksouk et al., 2015). Inhibition of histone deacetylases prevents deacetylation of key histones like histone 3 (H3) and histone 4 (H4), promoting open chromatin (euchromatin) and transcriptional activation. There are 11 distinct types of histone deacetylases, often categorized into class I, IIA, IIB, III and IV. Class III HDACs are not affected by any of the available HDACi and are often referred to as sirtuins (Sirt) which are known to mediate deacetylation of proteins like p53. Class I HDACs typically comprise HDACs 1, 2, 3 and 8; Class IIA include 4, 5, 7 and 9; Class IIB include 6 and 10, and Class IV is comprised of only HDAC 11 (Seto & Yoshida, 2014). Many studies have sought to characterize the various HDACi as being selective for one or another of these HDAC classes or specific isoforms, though at the relatively high plasma concentrations achieved in patients, the majority of the selectivity is lost. Romidepsin exhibits nanomolar potency against class I HDACs, while the hydroxamic acids generally have a broader pattern of activity and can be more accurately defined as pan-HDAC inhibitors, inhibiting Class I, II and IV (Bradner et al., 2010). While the Kd of any given HDACi against a particular isoform may vary, it is also clear that the profiles of genes activated or repressed by the different HDACi can vary significantly as a function of the HDACi, its concentration, its duration of exposure and the disease specific context. Efforts to ascribe inhibition of a particular HDAC to some predictive biomarker or outcome metric have been largely unsuccessful. As such, these drugs are often considered pleiotropic, and induce a broad spectrum of cellular effects. Complicating the mechanism of action further has been the recognition that HDACs can also deacetylate a host of non-histone proteins like Bcl-6, heat shock protein and others (Glozak et al., 2005). The implications of these effects in any given disease are presently unclear.

Despite the reproducible activity of these drugs in patients with R/R PTCL, a recent Phase IV post-marketing commitment of Romidepsin-CHOP vs. CHOP reported no difference in progression free survival (PFS) or overall survival (OS) between the arms, compelling the sponsor and U.S. FDA to withdrawal the PTCL indication for romidepsin (Bachy et al., 2022). This, coupled with the recognition that pralatrexate is now off patent in the U.S. is leading to reduced access to important drugs used to manage patients with R/R PTCL. In general, chemotherapy regimens, especially those modeled after the paradigms deployed to treat R/R aggressive B-cell malignancies, produce excessive toxicity with minimal clinical benefit in the T-cell lymphomas. As such, the options to treat patients with R/R PTCL are dwindling, leaving physicians and patients with few to no compelling alternatives.

Nanomedicine is a field that has been rapidly growing over recent years. Nanoparticle-based drug delivery systems offer the prospect of improving pharmacokinetic profile, tissue specificity and tumor penetration, resistance to premature enzymatic degradation, increased drug retention, and reduced systemic toxicities (Yao et al., 2020). Liposomes have been the most common method for generating therapeutic nanoparticles for anti-cancer applications. These drugs have generally been shown to have superior tolerability and efficacy, while also exhibiting improved pharmacologic features (Bozzuto & Molinari, 2015; Shi et al., 2017; Bourquin et al., 2018). However, liposomal lipids are subject to oxidation and liposomes face challenges in encapsulating therapeutic doses of many drugs, including hydrophobic ones and especially hydrophilic pharmaceuticals (Tenchov et al., 2021). The development of amphiphilic block co-polymer nanoparticles has markedly expanded the repertoire of drugs that can leverage the advantages of a nanoparticle mediated drug delivery (Gagliardi et al., 2021). These polymers offer biocompatibility, increased stability with superior versatility in the types and combinations of drugs that can be encapsulated. We sought to synthesize a nanoparticle of romidepsin that might overcome some of the historic liabilities associated with the drug, while capitalizing on the benefits of a novel nanoparticulate platform. Herein we report on the development of the first polymer nanoparticle (PNP) of romidepsin, Nanoromidepsin, demonstrating the superior properties of the molecule in contrast to the ‘naked’ version of romidepsin that has been used clinically for decades. Our approach was to design a di-block co-polymer nanoparticle with minimal excipients, in a highly controlled, scalable fabrication process using materials that are already used in clinical settings.

### SUMMARY

This summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter relates to compositions comprising, consisting essentially of, or a histone deacetylase inhibitor (HDACi) encapsulated in and/or otherwise associated with a nanoparticle, wherein the nanoparticle further comprises a detectable agent associated therewith, an additional therapeutic agent associated therewith, or a combination thereof. In some embodiments, the HDACi is selected from the group consisting of vorinostat, romidepsin, belinostat, and panobinostat, or any combination thereof, optionally wherein the HDACi is romidepsin. In some embodiments, the detectable agent comprises a fluorescent moiety, optionally wherein the detectable agent is selected from the group consisting of 3,3′-dioctadecyloxacarbocyanine perchlorate (DiO), rhodamine, indocyanine green, 1,1′-dioctadecyl-3,3,3′,3′-tetramethylindodicarbocyanine, 4-chlorobenzenesulfonate (DiD), and propidium iodide (PI). In some embodiments, the detectable agent comprises DiO. In some embodiments, the composition comprises one or more polymers and/or one or more surfactants, optionally wherein the one or more surfactants comprise a poloxamer, optionally poloxamer 188. In some embodiments, the one or more polymers are selected from the group consisting of a polyester, optionally poly-d,1-lactic acid (PDLLA) and/or poly(lactic acid; PLA), copolymers thereof, and blends thereof. In some embodiments, the polymer comprises a polymer selected from the group consisting of a synthetic polymer; a biodegradable polymer; a biocompatible polymer; an amphiphilic polymer; a diblock co-polymer; and blends thereof. In some embodiments, the polymer comprises a hydrophilic, PEG chain, optionally methoxy PEG, PEG-carboxylic acid, PEG-hydroxyl, and/or PEG amine as end cap and chain length range 0K-10K. In some embodiments, the polymer is a hydrophobic core-forming polymer, optionally a hydrophobic core-forming polymer selected from the group consisting of PDLLA and/or PLA. In some embodiments, the nanoparticle comprises a methyl ether-PEG poly-d,1-lactic acid. In some embodiments, one or more parameters selected from a group consisting of mode of phase addition, HDACi/polymer ratio, HDACi/surfactant ratio, solvent/anti-solvent ratio, rate of addition, and combinations thereof are optimized. In some embodiments, the HDACi/polymer ratio ranges from about 1:10 to about 1:100 W/W, optionally 1:10 to about 1:50 W/W; the HDACi/surfactant ratio ranges from about 1:0.05 to about 1:0.2 W/W; the solvent/anti-solvent ratio ranges from about 1:10 to about 1:1, optionally wherein the anti-solvent is selected from the group consisting of water, PBS, or another ionic buffer solution; and/or the rate of addition ranges from about 10 to about 500 mL/hour, optionally about 10 to about 50 mL/hour.

In some embodiments, the presently disclosed subject matter also relates to methods for treating diseases, disorders, and/or conditions associated with sensitivity to histone deacetylase inhibitors. In some embodiments, the methods comprise administering to a subject in need thereof an effective amount of a composition as disclosed herein. In some embodiments, the disease, disorder, and/or condition associated with sensitivity to an HDACi is a tumor and/or a cancer, an inflammatory disease, disorder, or condition; an autimmune disease, disorder, or condition; or any combination thereof. In some embodiments, the tumor and/or the cancer is selected from the group consisting of cutaneous T cell lymphoma (CTCL), peripheral T cell lymphoma (PTCL), multiple myeloma, large granular lymphocytic leukemia (LGLL), and adult T cell leukemia/lymphoma.

In some embodiments, the presently disclosed subject matter also relates to methods for inhibiting the growth, proliferation, and/or metastasis of tumors and/or cancers associated with sensitivity to a histone deacetylase inhibitors. In some embodiments, the methods comprising administering to a subject in need thereof an effective amount of a nanoparticle composition as disclosed herein. In some embodiments, the tumor and/or the cancer is selected from the group consisting of cutaneous T cell lymphoma (CTCL), peripheral T cell lymphoma (PTCL), multiple myeloma, large granular lymphocytic leukemia (LGLL), and adult T cell leukemia/lymphoma. In some embodiments, the presently disclosed methods further comprise administering to the subject at least one additional therapeutically active agent. In some embodiments, the at least one additional therapeutically active agent is a chemotherapeutic agent. In some embodiments, the chemotherapeutic agent is selected from the group consisting of acitretin, bexarotene, an interferon, optionally an alpha and/or a gamma interferon, cyclosporine A, methotrexate, romidepsin, vorinostat, cyclophosphamide, doxorubicin, vincristine, prednisone, dexamethasone, etoposide, vincristine, brentuximab vedotin, a histone lysine methyltransferase (HMT) enhancer of zeste homolog 1 (EZH1) inhibitor, a histone lysine methyltransferase (HMT) enhancer of zeste homolog 2 (EZH2) inhibitor, a JAK/STAT pathway inhibitor, optionally ruxolitinib, tofacitinib, baricitinib, fedratinib, upadacitinib, abrocitinib, and/or deucravacitinib; a hypomethylating agent, an immunotherapeutic agent, optionally a monoclonal antibody, further optionally alemtuzumab; ranimustine, vindesine, carboplatin, and combinations thereof. In some embodiments, the method comprises a combination therapy comprising a combination of active agents selected from the group consisting of cyclophosphamide/doxorubicin/vincristine/prednisone (CHOP); eoposide/vincristine/doxorubicin/cyclophosphamide/prednisone (CHOEP); clophosphamide/doxorubicin/prednisone/brentuximab vedotin (CHP-BV); brentuximab vedotin/cyclophosphamide/doxorubicin/prednisone (CHP); and vincristine/cyclophosphamide/doxorubicin/prednisone (VCAP) plus doxorubicin/ranimustine/prednisone (AMP), and vindesine/etoposide/carboplatin/prednisone (VECP; VCAP-AMP-VECP).

In some embodiments, the presently disclosed subject matter also relates to methods for treating inflammatory and/or an autoimmune diseases, disorders, and/or conditions associated with sensitivity to histone deacetylase inhibitors. In some embodiments, the methods comprise administering to a subject in need thereof an effective amount of a nanoparticle composition as disclosed herein. In some embodiments, the inflammatory and/or an autoimmune disease, disorder, or condition is selected from the group consisting of fatty liver disease, endometriosis, types 1 and 2 diabetes, inflammatory bowel disease, asthma, obesity, Alzheimer's and Parkinson's diseases, Ankylosing Spondylitis (AS), Antiphospholipid Antibody Syndrome (APS), Gout, Inflammatory Arthritis Center, Myositis, Rheumatoid Arthritis, Scleroderma, Sjogren's Syndrome, Systemic Lupus Erythematosus (SLE, Lupus), vasculitis, Addison's disease, Celiac disease-sprue (gluten-sensitive enteropathy), dermatomyositis, Grave's disease, Hashimoto's thyroiditis, Multiple sclerosis, Myasthenia gravis, Pernicious anemia, Reactive arthritis, Psoriasis/psoriatic arthritis, multiple sclerosis, Systemic lupus erythematosus (SLE), type 1 diabetes, Inflammatory bowel disease (including Crohn's disease and ulcerative colitis), autoimmune vasculitis, Guillain-Barre syndrome, and Chronic inflammatory demyelinating polyneuropathy. In some embodiments, the presently disclosed methods further comprise administering to the subject at least one additional therapeutically active agent. In some embodiments, the at least one additional therapeutically active agent is an anti-inflammatory and/or an immunosuppresant agent.

In some embodiments, the presently disclosed subject matter also relates to methods for imaging cells, tissues, organs, and/or other targets in subjects. In some embodiments, the methods comprise administering to the subject a composition comprising a histone deacetylase inhibitor (HDACi) encapsulated in and/or otherwise associated with a nanoparticle in an amount and via a route such that a detectable quantity of the composition comes in contact with the cell, tissue, organ, or other target in the subject and preferentially accumulates there, wherein the nanoparticle further comprises a detectable agent associated therewith; and detecting a presence of the nanoparticle in and/or on the cell, tissue, organ, or other target in the subject using an imaging system, whereby the cell, tissue, organ, or other target in the subject is imaged in the subject. In some embodiments, the HDACi is selected from the group consisting of vorinostat, romidepsin, belinostat, and panobinostat, or any combination thereof, optionally wherein the HDACi is romidepsin. In some embodiments, the detectable agent comprises a fluorescent moiety, optionally wherein the detectable agent is 3,3′-dioctadecyloxacarbocyanine perchlorate (DiO), rhodamine, indocyanine green, DiD, and PI. In some embodiments, the composition comprises one or more polymers and/or one or more surfactants, optionally wherein the one or more surfactants comprise a poloxamer, optionally poloxamer 188. In some embodiments. the one or more polymers are selected from the group consisting of a polyester, optionally poly-d,1-lactic acid (PDLLA) and/or poly(lactic acid; PLA), copolymers thereof, and blends thereof. In some embodiments, the polymer comprises a polymer selected from the group consisting of a synthetic polymer; a biodegradable polymer; a biocompatible polymer; an amphiphilic polymer; a diblock co-polymer; and blends thereof. In some embodiments, the polymer comprises a hydrophilic, PEG chain, optionally methoxy PEG, PEG-carboxylic acid, PEG-hydroxyl, and/or PEG amine as end cap and chain length range 0K-10K. In some embodiments, the polymer is a hydrophobic core-forming polymer, optionally a hydrophobic core-forming polymer selected from the group consisting of PDLLA and/or PLA. In some embodiments, the nanoparticle comprises a methyl ether-PEG poly-d,1-lactic acid (mPEG-PDLLA), a methyl ether-PEG polylactic acid (mPEG-PLA), or any combination thereof.

The compositions and methods of the presently disclosed subject matter are appropriate for use in medical applications (e.g., diagnostic and/or treatment applications). In some embodiments, the compositions and methods of the presently disclosed subject matter are wherein the subject is a mammal, optionally a human or a veterinary animal.

Accordingly, it is an object of the presently disclosed subject matter to provide compositions that include histone deacetylase (HDAC) inhibitors encapsulated in and/or otherwise associated with detectable nanoparticles, and methods for using the same in medical and veterinary applications.

This and other objects are achieved in whole or in part by the presently disclosed subject matter. Further, an object of the presently disclosed subject matter having been stated above, other objects and advantages of the presently disclosed subject matter will become apparent to those skilled in the art after a study of the following description, Figures, and EXAMPLES, which are incorporated by reference and form part of the specification.

### BRIEF DESCRIPTIONS OF THE FIGURES

The patent or application file contains at least one drawing executed in color. Copies of this patent or patent application publication with color drawing(s) will be provided by the Office upon request and payment of the necessary fee.

FIGS. 1A-1J: Romidepsin nanoparticle synthesis, Physicochemical characterization, and drug activity analysis in PTCL cells in vitro. (FIG. 1A) Carrier selection screening. Romidepsin encapsulation quantified by LC/MS (FIG. 1B) Cryo-EM of (i) Ghost in H2O (ii) Nanoromidepsin in H2O (iii) Nanoromidepsin in PBS (FIG. 1C) DLS graphs (top) and Zeta-potential (bottom) spectra of Nanoromidepsin in H₂O (FIG. 1D) DLS and Zeta potential data of Nanoromidepsin ghost and Nanoromidepsin in H₂O. (FIG. 1E) HH and H9 (CTCL), SUP-M2 (ALK+ALCL) TL1 (LGL-leukemia), NKL (NK cell lymphoblastic leukemia/lymphoma), FM3-29 (melanoma) were treated with free romidepsin and different analogs of Nanoromidepsin (mPEG-PDLLA Nanoromidepsin-H₂O, mPEG-PDLLA Nanoromidepsin-PBS and mPEG-PLGA Nanoromidepsin-H₂O. The cytotoxicity was determined using CELLTITER-GLO® assay after 60 hours of treatment. (FIG. 1F) IC₅₀ (nM) for romidepsin and Nanoromidepsin analogs for the 8 cell lines at 60 hours. Flow cytometry and western blot analysis of (FIG. 1G) Ac-H3-lys27, and Ac-H4-lys16 (FIG. 1H) cleaved PARP expressing in HH cell line after 30 hours of treatment with indicated treatment of increasing concentration of free romidepsin and Nanoromidepsin. Data presented as mean±SD. (FIG. 1I) Western blot analysis of Ac-H3-lys27, Ac-H4-lys16, and cleaved PARP at 24 hours after treatment with Ghost, romidepsin, and Nanoromidepsin mPEG-PDLLA H₂O (FIG. 1J) densitometry analysis of the Western blot analysis.

FIGS. 2A-2F: Scale-up schematic of Romidepsin polymer nanoparticle (Nanoromidepsin) synthesis, physicochemical characterization, and drug activity analysis in PTCL cells in vitro. (FIG. 2A) Schematic representation of Nanoromidepsin fabrication (FIG. 2B) The statistical variability of ten, scale up (20 mL or greater) batches. Of note, these were formulated with different material stocks (polymer, Romidepsin, solvent, etc.). (FIG. 2C) Stability of Nanoromidepsin with respect to (i) size and PDI (ii) concentration. (FIG. 2D) Cryo-EM images of coloaded Nanoromidepsin with DiO and Ghost DiO. (FIG. 2E) HH and H9 (CTCL), SUPM2 and SUDHL-1 (ALK+ALCL) TL1 (LGL-leukemia), NKL (NK cell lymphoblastic leukemia/lymphoma) were treated with free romidepsin and different analogs of Nanoromidepsin (mPEG-PDLLA Nanoromidepsin H₂O and mPEG-PDLLA Nanoromidepsin PBS). The cytotoxicity was determined using CELLTITER-GLO® assay after 48 hours of treatment. (FIG. 2F) Western blot analysis of PARP, Ac-H3-K9, and Ac-H4-K16 in HH, H9, TL1 and SUP-M2 cell lines after 48 hours of treatment with indicated treatment of increasing concentration of free romidepsin and different analogs of Nanoromidepsin (NanoRomi water and NanoRomi PBS).

FIGS. 3A-3E: Effect of Nanoromidepsin on Primary LGL Leukemia patient PBMC samples. (FIG. 3A) Freshly frozen PBMCs from LGL leukemia patients were treated with indicated doses of romidepsin (solid line) or Nanoromidepsin (dotted line) for 48 hours. The cytotoxicity was determined using CELLTITER-GLO® assay after 48 hours of treatment. (FIG. 3B) IC50 (nM) for romidepsin and Nanoromidepsin for 10 LGL leukemia patients at 48 hours. (FIG. 3C) PBMCs from patients with LGL leukemia and healthy donor control were screened by flow cytometry. The lymphocyte and singlet cell gating were performed as described earlier. The CD3+/CD8+/CD57+/− cells were gated from singlet lymphocyte population as indicated. The cleaved PARP or viability dye staining was analyzed in CD3+/CD8+/CD57+ or CD3+/CD8+/CD57− cells as indicated. The flow images were generated from a representative LGL patient (PT No. 03) PBMC sample treated with DMSO or romidepsin (10 nM). Ghost or Nanoromidepsin treated samples were similarly analyzed. (FIG. 3D) Cleaved PARP (apoptosis) and (FIG. 3E) live-dead dye staining (cell viability) after the incubation with free romidepsin and Nanoromidepsin for 48 hours. Data presented as percentage CD3+/CD8+/ CD57+ (more differentiated LGL) or CD3+/CD8+/CD57− (less differentiated LGL) cells positive for cleaved PARP or live-dead dye staining. The data presented after subtracting spontaneous apoptosis or cell viability values from the DMSO-treated controls.

FIGS. 4A-4F: Pharmacokinetics and tissue distribution of Nanoromidepsin in vivo. (FIG. 4A) Plasma concentration-time dependence plot of romidepsin concentration in plasma after intraperitoneal or intravenous administration of a single treatment with free romidepsin or Nanoromidepsin (FIG. 4B) diagram representing experimental time-points associated with Nanoromidepsin co-loaded with a fluorescent dye DiO or free DiO administration and fluorescent images evaluation, as well as organs collection. (FIG. 4C) fluorescence images of H9-dTomato-luc tumor-bearing mice taken at different time points after intravenous injection of free DiO or DiO and romidepsin encapsulated nanoparticle (NanoromiDiO; FIG. 4D) ex vivo fluorescence images and (FIG. 4E) corresponding optical intensity of tumor and major organs (tumor, liver, spleen, kidney, heart, and lung, respectively) dissected at 72 hours post-injection. Statistical significance was determined by using student t test (Mann-Whitney) where *p<0.05; **p<0.01; ***p<0.001. (FIG. 4F) Mice bearing H9-dtomato-luc xenograft were treated with 4 mg/kg romidepsin and Nanoromidepsin. After 24 hours, tumors (n=3) were collected for LCMS based quantification of romidepsin in tumor tissue.

FIGS. 5A-5I: Tolerability of free romidepsin and Nanoromidepsin in vivo. BALB/c mice were administered a single treatment of indicated doses of romidepsin and Nanoromidepsin by (FIG. 5A) intraperitoneal (FIG. 5B) intravenous route of administration. Tolerability of various doses of free romidepsin and Nanoromidepsin in BALB/c mice was assessed by monitoring body weight and overall health conditions after a single treatment. X represents dead mice. (FIG. 5C) Mice were administered a 4 mg/kg treatment of romidepsin or Nanoromidepsin by intravenous route of administration for arrow indicated days (1, 8, and 15 days) in H9-dTmatoluc xenograft bearing NSG mice. Depicted are the percentage of body weight changes as a percentage of starting weights with SEM (FIG. 5D) clinical score. Mice were administered an 8 mg/kg treatment of romidepsin or Nanoromidepsin by intravenous route of administration for arrow indicated days (1 and 15 days) in H9-dTmato-luc xenograft bearing NSG mice. Depicted are the (FIG. 5E) percentage of body weight changes as a percentage of starting weights with SEM and (FIG. 5F) clinical score as defined in Materials and Methods. (FIG. 5G) The hepatic parenchyma from ghost, romidepsin, and Nanoromidepsin treated mice display normal microarchitecture, without evidence of drug-induced liver injury (original magnifications X200; H&E stain). Soft tissue-based tumors from ghost-treated mice contain sheet-like infiltrates of large atypical lymphocytes with pleomorphic nuclei, distinct nucleoli and amphophilic cytoplasm; mitotic activity is brisk (original magnification X1000; H&E stain). Romidepsin and Nanoromidepsin-treated tumors are associated with varying degrees of treatment-related necrosis (original magnifications X1000; H&E stain). Red arrows mark mitotic figures and black arrow indicates necrosis/apoptosis. (FIG. 5H) LC-MS based quantification of liver (FIG. 5I) LC-MS based quantification of plasma collected from 4C experiment after 1 and 24 hours. Statistical significance was determined by using student t test (Mann-Whitney) where *p<0.05; ** p<0.01; ***, p<0.001.

FIGS. 6A-6E: Nanoromidepsin showed superior activity but similar survival rate compared to free romidepsin in TCL xenograft bearing NSG mice. (FIG. 6A) Diagram representing the inoculation and dosing schedule of free romidepsin and Nanoromidepsin in H9-dtomato-luc xenograft bearing mice (FIG. 6B) and (FIG. 6D) Region-of-interest analysis of BLIs (readout for tumor growth) from different treatment groups were recorded at various time points over the course of 8 weeks. Statistical significance was determined by using student t test (Mann-Whitney) where *p<0.05; ** p<0.01; *** p<0.0001 (FIG. 6C) Survival curves for romidepsin-treated, Nanoromidepsin-treated, ghost nanoparticle and control mice (n=9 per group). The arrows indicate treatment days. Statistical significance was determined by using log rank test where *p<0.05; **p<0.01; ***p<0.0001 (FIG. 6E) Whole-body bioluminescence images of H9-dTomato-luc xenograft-bearing mice taken at the indicated day. Red box indicates dead mouse.

FIGS. 7A-7D: Dosing schedule change of Romidepsin encapsulated nanoparticle showed superior activity and survival rate compared to free romidepsin in CTCL xenograft bearing NSG mice. (FIG. 7A) Diagram representing the inoculation and dosing schedule of free romidepsin and Nanoromidepsin in TCL xenograft bearing mice (FIG. 7B) Region-of-interest analysis of BLIs (readout for tumor growth) from different treatment groups at various time points during the course of treatment and plotted as bar graph. (FIG. 7C) Whole-body bioluminescence images of H9-dTomatoluc xenograft-bearing mice taken at the indicated day. (FIG. 7D) Survival curves for romidepsin-treated, Nanoromidepsin-treated, ghost nanoparticle and control mice (n=9 per group). The arrows indicate treatment days. Statistical significance was determined by using log rank test where *p<0.05; **p<0.01; ***p<0.0001.

FIG. 8. HH (CTCL), SUP-M2 (ALK+ALCL) TL1 (LGL-leukemia), NKL (NK cell lymphoblastic leukemia/lymphoma), FM3-29 (melanoma) were treated with different analogs of ghost PNP. The cytotoxicity was determined using CELLTITER-GLO® assay after 60 hours of treatment.

FIG. 9. IC₅₀ (nM) for romidepsin and Nanoromidepsin analogs for the 8 cell lines at 48 hours.

FIG. 10. Toxicity of free romidepsin and Nanoromidepsin in mice: BALB/c mice were administered a single treatment of indicated doses of romidepsin and Nanoromidepsin by (upper panel) intraperitoneal; (lower panel) intravenous route of administration. Tolerability of various doses of free romidepsin and Nanoromidepsin in balb/c mice was assessed by monitoring clinical score after a single treatment.

FIG. 11. Mice were administered a treatment of romidepsin or Nanoromidepsin or ghost PNP by intraperitoneal route of administration for 1, 8 and 15 days in H9-dTmato-luc xenograft bearing NSG mice. Depicted are the percentage of body weight changes as a percentage of starting weights with SEM and clinical score. X represents dead mice.

FIG. 12. Mice were administered a 2 and 3 mg/kg treatment of romidepsin or Nanoromidepsin or ghost PNP by intravenous route of administration for arrow indicated days (1, 4, 8 and 11 days) in H9-dTmato-luc xenograft bearing NSG mice. Depicted are the percentage of body weight changes as a percentage of starting weights with SEM and clinical score. X represents dead mice.

FIG. 13. Mice were administered a 2.5 and 5 mg/kg treatment of romidepsin or Nanoromidepsin or ghost PNP by intravenous route of administration for arrow indicated days in H9-dTmato-luc xenograft bearing NSG mice. Depicted are the percentage of body weight changes as a percentage of starting weights with SEM and clinical score. X represents dead mice.

### DETAILED DESCRIPTION

### I. Definitions

The terminology used herein is for the purpose of describing particular embodiments only and is not intended to be limiting of the presently disclosed subject matter.

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

In describing the presently disclosed subject matter, it will be understood that a number of techniques and steps are disclosed. Each of these has individual benefit and each can also be used in conjunction with one or more, or in some cases all, of the other disclosed techniques.

Accordingly, for the sake of clarity, this description will refrain from repeating every possible combination of the individual steps in an unnecessary fashion. Nevertheless, the specification and claims should be read with the understanding that such combinations are entirely within the scope of the presently disclosed and claimed subject matter.

Following long-standing patent law convention, the terms “a”, “an”, and “the” refer to “one or more” when used in this application, including in the claims. Similarly, the phrase “at least one”, when employed herein to refer to an entity, refers to, for example, 1, 2, 3, 4, 5, 6, 7, 8, 9, 10, 15, 20, 25, 30, 35, 40, 45, 50, 75, 100, or more of that entity, including but not limited to whole number values between 1 and 100 and in some embodiments greater than 100.

Unless otherwise indicated, all numbers expressing quantities of ingredients, reaction conditions, and so forth used in the specification and claims are to be understood as being modified in all instances by the term “about”. The term “about”, as used herein when referring to a measurable value such as an amount of mass, weight, time, volume, concentration, or percentage, is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods and/or employ the disclosed compositions. Accordingly, unless indicated to the contrary, the numerical parameters set forth in this specification and attached claims are approximations that can vary depending upon the desired properties sought to be obtained by the presently disclosed subject matter.

Numerical ranges recited herein by endpoints include all numbers and fractions subsumed within that range (e.g., 1 to 5 includes 1, 1.5, 2, 2.75, 3, 3.90, 4, 4.24, and 5). Similarly, numerical ranges recited herein by endpoints include subranges subsumed within that range (e.g., 1 to 5 includes 1-1.5, 1.5-2, 2-2.75, 2.75-3, 3-3.90, 3.90-4, 4-4.24, 4.24-5, 2-5, 3-5, 1-4, and 2-4).

A disease or disorder is “alleviated” if the severity of a symptom of the disease, condition, or disorder, or the frequency at which such a symptom is experienced by a subject, or both, are reduced.

As used herein, the term “and/or” when used in the context of a list of entities, refers to the entities being present singly or in combination. Thus, for example, the phrase “A, B, C, and/or D” includes A, B, C, and D individually, but also includes any and all combinations and subcombinations of A, B, C, and D.

The terms “additional therapeutically active compound” and “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease, or disorder being treated.

As used herein, the term “adjuvant” refers to a substance that elicits an enhanced immune response when used in combination with a specific antigen.

As use herein, the terms “administration of” and/or “administering” a compound should be understood to refer to providing a compound of the presently disclosed subject matter to a subject in need of treatment.

The term “comprising”, which is synonymous with “including” “containing”, or “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting essentially of” limits the scope of the related disclosure or claim to the specified materials and/or steps, plus those that do not materially affect the basic and novel characteristic(s) of the disclosed and/or claimed subject matter. For example, a pharmaceutical composition can “consist essentially of” a pharmaceutically active agent or a plurality of pharmaceutically active agents, which means that the recited pharmaceutically active agent(s) is/are the only pharmaceutically active agent(s) present in the pharmaceutical composition. It is noted, however, that carriers, excipients, and/or other inactive agents can and likely would be present in such a pharmaceutical composition and are encompassed within the nature of the phrase “consisting essentially of”.

As used herein, the phrase “consisting of” excludes any element, step, or ingredient not specifically recited. It is noted that, when the phrase “consists of” appears in a clause of the body of a claim, rather than immediately following the preamble, it limits only the element set forth in that clause; other elements are not excluded from the claim as a whole.

With respect to the terms “comprising”, “consisting of”, and “consisting essentially of”, where one of these three terms is used herein, the presently disclosed and claimed subject matter can include the use of either of the other two terms. For example, a composition that in some embodiments comprises a given active agent also in some embodiments can consist essentially of that same active agent, and indeed can in some embodiments consist of that same active agent.

“Amphiphilic polymers” as used herein, describe polymer materials comprising both hydrophilic and hydrophobic unit chains. In some embodiments, various polymers control NP properties, polymers include but are not limited to m-PEG-PDLLA and m-PEG-PLA of various respective chain lengths of hydrophobic core and PEG, which can confer a “stealth” property.

The term “aqueous solution” as used herein can include other ingredients commonly used, such as sodium bicarbonate described herein, and further includes any acid or base solution used to adjust the pH of the aqueous solution while solubilizing a peptide.

“Batch-to-batch” as used herein, describes the manner by which the formulation is reproducible, with optimal variation between batches in the context of physio-chemical properties especially for drug loading.

The term “binding” refers to the adherence of molecules to one another, such as, but not limited to, enzymes to substrates, ligands to receptors, antibodies to antigens, DNA binding domains of proteins to DNA, and DNA or RNA strands to complementary strands.

“Binding partner”, as used herein, refers to a molecule capable of binding to another molecule.

The term “biocompatible”, as used herein, refers to a material that does not elicit a substantial detrimental response in the host.

“Biodegradable” as used herein, refers to the materials that can break down inside the body to non-toxic natural products and can be easily eliminated. In general, biocompatible and biodegradable are often associated with PLA polymers comprising ester bonds. In some embodiments, the breakdown of these polymers is due to cellular or in vivo biological actions not by hydrolysis. The polymers with biodegradable properties play a role in the drug release. In some embodiments, drug release is governed by cleavage of polymer bonds, erosion of polymer matrix, and diffusion of encapsulated drug from the particles.

As used herein, the terms “biologically active fragment” and “bioactive fragment” of a peptide encompass natural and synthetic portions of a longer peptide or protein that are capable of specific binding to their natural ligand and/or of performing a desired function of a protein, for example, a fragment of a protein of larger peptide which still contains the epitope of interest and is immunogenic.

The term “biological sample”, as used herein, refers to samples obtained from a subject, including but not limited to skin, hair, tissue, blood, plasma, cells, sweat, and urine.

“Centrifugal filters” as used herein, describe the materials used to process nanoparticles to remove excess unencapsulated drug by centrifugation or ultracentrifugation methods. In some embodiments, a centrifugal filter that is employed in the methods of the presently disclosed subject matter has a cutoff range of about 3K to about 100K.

A “coding region” of a gene comprises the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein with reference to drugs and drug interactions refers to an interaction or interactions among one or more therapeutic agents that results in a greater benefit to a subject than would have occurred if the one or more therapeutic agents were not given to the subject. In some embodiments, a complementary drug interaction results in a synergistic benefit to the subject.

“Complementary” as used herein with reference to biomolecules refers to the broad concept of subunit sequence complementarity between two nucleic acids (e.g., two DNA molecules). When a nucleotide position in both of the molecules is occupied by nucleotides normally capable of base pairing with each other at a given position, the nucleic acids are considered to be complementary to each other at this position. Thus, two nucleic acids are complementary to each other when a substantial number (in some embodiments at least 50%) of corresponding positions in each of the molecules are occupied by nucleotides that can base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. By way of example and not limitation, the first region comprises a first portion and the second region comprises a second portion, whereby, when the first and second portions are arranged in an antiparallel fashion, in some embodiments at least about 50%, in some embodiments at least about 75%, in some embodiments at least about 90%, and in some embodiments at least about 95% of the nucleotide residues of the first portion are capable of base pairing with nucleotide residues in the second portion. In some embodiments, all nucleotide residues of the first portion are capable of base pairing with nucleotide residues in the second portion.

A “compound”, as used herein, refers to a polypeptide, an isolated nucleic acid, a small molecule, or other agent used in the compositions and/or methods of the presently disclosed subject matter.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a condition, disease, or disorder for which the test is being performed.

A “test”cell is a cell being examined.

A “pathoindicative” cell is a cell that, when present in a tissue, is an indication that the animal in which the tissue is located (or from which the tissue was obtained) is afflicted with a condition, disease, or disorder.

A “pathogenic” cell is a cell that, when present in a tissue, causes or contributes to a condition, disease, or disorder in the animal in which the tissue is located (or from which the tissue was obtained).

A tissue “normally comprises” a cell if one or more of the cell are present in the tissue in an animal not afflicted with a condition, disease, or disorder.

“Combinatorial” and “combinatorial fashion” as used herein, describes the process of utilizing multiple parameters in a single experiment to compare the physiochemical properties of nanoparticles.

“Concentration”, as used herein, is a measurement of the quantity of drug in the nanoparticles. In general, concentration of drug in nanoparticles is impacted by both mechanical process and formulation process, including solvent and anti-solvent ratio, drug to polymer ratio, mixing speed, solvent properties including density, D, dielectric constant, polarity, viscosity, eluent strength, etc. In some embodiments, particles can contain romidepsin in a concentration including, but not limited to 20μg/ml to 1000μg/mL.

As used herein, the terms “condition”, “disease condition”, “disease”, “disease state”, and “disorder” refer to physiological states in which diseased cells or cells of interest can be targeted with the compositions of the presently disclosed subject matter. In some embodiments, a disease is T cell lymphoma. In some embodiments, a disease is a tumor and/or a cancer, optionally a cancer of the blood. In some embodiments, a disease is large granular lymphocyte leukemia.

“Controlled addition” as used herein, describes the addition of one phase to another phase by fixed rate of addition (in some embodiments, from about 10 to about 500 mL/hour) by using a syringe pump to produce particles with reproducible properties including but not limited to size, PDI, zeta potential, and drug loading.

“Cryo-protectant”, as used herein, describes the excipient or a stabilizer to protect the stability of NPs during the lyophilization process, including but not limited to an excipient such as mannitol, glucose, and the like.

As used herein, the term “diagnosis” refers to detecting a risk or propensity to a condition, disease, or disorder. In any method of diagnosis exist false positives and false negatives. Any one method of diagnosis does not provide 100% accuracy.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” or “therapeutically effective amount” refers to an amount of a compound or composition sufficient to produce a selected effect, such as but not limited to alleviating symptoms of a condition, disease, or disorder. In the context of administering compounds in the form of a combination, such as multiple compounds, the amount of each compound, when administered in combination with one or more other compounds, may be different from when that compound is administered alone. Thus, an effective amount of a combination of compounds refers collectively to the combination as a whole, although the actual amounts of each compound may vary. The term “more effective” means that the selected effect occurs to a greater extent by one treatment relative to the second treatment to which it is being compared.

“Encapsulation efficiency” as used herein, designates % of drug encapsulated with in the particle compared to the total drug used.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA, and mRNA) or a defined sequence of amino acids and the biological properties resulting therefrom. Thus, a gene encodes a protein if transcription and translation of an mRNA corresponding to or derived from that gene produces the protein in a cell or other biological system and/or an in vitro or ex vivo system. Both the coding strand, the nucleotide sequence of which is identical to the mRNA sequence (with the exception of uracil bases presented in the latter) and is usually provided in Sequence Listing, and the non-coding strand, used as the template for transcription of a gene or cDNA, can be referred to as encoding the protein or other product of that gene or cDNA.

As used herein, an “essentially pure” preparation of a particular protein or peptide is a preparation wherein in some embodiments at least about 95% and in some embodiments at least about 99%, by weight, of the protein or peptide in the preparation is the particular protein or peptide.

“Formulation variables” as used herein describe a variable or parament to be considered for modification in a reaction mixture for a nanoparticle, including but not limited to drug to polymer ratio, choice of surfactant, surfactant addition to solvent or nonsolvent, water to organic ratio, a parameter that contributes to improvement of encapsulation efficiency.

A “fragment”, “segment”, or “subsequence” is a portion of an amino acid sequence, comprising at least one amino acid, or a portion of a nucleic acid sequence comprising at least one nucleotide. The terms “fragment”, “segment”, and “subsequence” are used interchangeably herein. In some embodiments, a “fragment”, “segment”, or “subsequence” of a biomolecule (e.g., a peptide, polypeptide, or nucleic acid) is characterized by a level of a particular biological activity relative to the full length biologically active biomolecule to which it is related, although it is understood that the level of the activity need not be identical to that of the full length biologically active biomolecule to which it is related provided that the level of activity satisfies a desired use of the “fragment”, “segment”, or “subsequence”.

As used herein, a “functional” biological molecule is a biological molecule in a form in which it exhibits a property by which it can be characterized. A functional enzyme, for example, is one that exhibits the characteristic catalytic activity by which the enzyme can be characterized.

“Hydrophilic” as used herein, refers to substances containing high polar groups that readily interact with and/or are soluble in water.

“Hydrophobic” as used herein, refers to substances containing less polar groups and are typically characterized by low solubility in water.

As used herein, the phrases “inhibitor of HDAC activity”, “HDAC inhibitors (HDACi)”, and grammatical variants thereof refer to inhibitors of at least one biological activity of at least one histone deacetylase (HDAC), whether in vitro, in vivo, or ex vivo. Exemplary HDAC inhibitors include, but are not limited to romidepsin, vorinostat, panobinostat, belinostat, and chidamide (i.e., N-(2-Amino-4-fluorophenyl)-4-[[[(E)-3-pyridin-3-ylprop-2-enoyl]amino]methyl]benzamide). Other compounds that can serve as HDAC inhibitors include valproic acid, trichostatin A, butyric acid and its derivatives including but not limited to 4-phenylbutyric acid, entinostat, givinostat, droxinostat, tubastatin A, pracinostat, and others.

As used herein “injecting”, “applying”, and administering” include administration of a compound of the presently disclosed subject matter by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

As used herein, a “ligand” is a compound that specifically binds to a target compound or molecule. A ligand “specifically binds to” or “is specifically reactive with” a compound when the ligand functions in a binding reaction which is determinative of the presence of the compound in a sample of heterogeneous compounds.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, such as but not limited to through ionic or hydrogen bonds or van der Waals interactions.

“Lyophilization” as used herein, describes the process utilized to create powder form of NPs with or without cryo-protectants to improve the shelf-life, dosage logistics, and storage logistics.

“Mean particle size” used herein, generally refers in some embodiments to spherical particles' hydrodynamic diameter. In general, NP size plays a role in macrophage uptake over a surface chemistry, such as a PEG surface chemistry. In some embodiments, a lower molecular weight of a polymer used to prepare a NP contributes to smaller size NPs resulting in altered drug release kinetics, higher circulation, less accumulation in organs like liver and spleen, larger exposure of drug contributes to enhanced biological activity.

The terms “measuring the level of expression” and “determining the level of expression” as used herein refer to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

The phrase “molecular weight”, as used herein, refers to the chain length of bulk polymer. In some embodiments, physical properties like solubility, viscosity, crystallinity, mechanical strength, and degradation rate can depend on the molecular weight of the polymer.

“Monodisperse” and homogenous solutions are used as synonyms or interchangeably, used herein, particles of same or almost same diameter distribution is referred as monodisperse.

“Multi-variant” as used herein, describes the number of variables included into the preparation of NPs that determines the physio-chemical properties of the drug.

The terms “nano”, “nanomaterial”, “nanoparticle”, and “NP” as used herein, refer to a structure having at least one region with a dimension and/or size (e.g., length, width, diameter, etc.) less than or equal to about 1,000 nm including all integers or fractional integers in between (such as but not limited to 30, 40, 50, 60, 70, 80, 90, 100, 200, 500, 1,000 nm). In some embodiments, the dimension is smaller (e.g., less than about 500 nm, less than about 250 nm, less than about 200 nm, less than about 150 nm, less than about 125 nm, less than about 100 nm, less than about 80 nm, less than about 70 nm, less than about 60 nm, less than about 50 nm, less than about 40 nm, less than about 30 nm or even less than about 20 nm). In some embodiments, the dimension is between about 20 nm and about 250 nm (e.g., about 20, 30, 40, 50, 60, 70, 80, 90, 100, 110, 120, 130, 140, 150, 160, 170, 180, 190, 200, 210, 220, 230, 240, or 250 nm). In general, particles occupy size ranges between that which induce size-dependent rapid renal clearance and prevent size-dependent liver accumulation. The present disclosed compositions might exist in a variety of shapes, including, but not limited to core-shell, circular, spherical, spheroidal, and micellar. In general, nanoparticles having a spherical shape are referred to as Nano spheres.

In some embodiments, nanoparticles, including “core-shell” nanoparticles, are formed from biocompatible, biodegradable, block co-polymers in amphiphilic nature. In some embodiments, a core-shell nanoparticle is a nanoparticle made out of di-block polymers wherein the shell is hydrophilic and the core is hydrophobic.

“Nanoprecipitation” as used herein refers in some embodiments to the method of making polymer NPs by bottom-up approach. In some embodiments of this method, one phase is added to another phase under moderate magnetic stirring. In some embodiments, nanoprecipitation is employed to encapsulate hydrophobic drugs. It is chosen because of its simplicity, scalability, and batch-to-batch reproducibility by controlled addition. This method facilitates multi-parameter optimization in a combinatorial fashion to achieve the desired properties such as size, zeta potential, and drug loading.

“Operating variables” as used herein, describe a mechanical variable or parament to be considered for modification including, but not limited to mechanical speed, mixing solvents, mode of addition, rate of addition, centrifugation speed, and time to improve the physiochemical properties of colloidal solution.

“Optimization” as used herein, describes the process of finding a desirably effective drug concentration in particles to exert therapeutic effect. In some embodiments, a multi-pronged approach is used to optimize drug concentration by including process and formulation parameters in a combinatorial fashion. “Multi-pronged” as used herein, describes the process of approach to engineering the nanoprecipitation method to optimize the physiochemical properties of Nanoparticles. In general, nanoprecipitation method facilitates parameter optimization. In other embodiments, a syringe pump with a multi-channel syringe system is applied to engineer nanoparticles.

The term “otherwise identical sample”, as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

“Parallel” as used herein refers to an approach for nanoparticle fabrication involving multi-parameter optimization in a combinatorial fashion and it is often used as synonym of combinatorial approach.

“Parameter” as used herein, describes a parameter to be considered for modification with respect to physiochemical properties of NPs. In general, there are process and formulation related parameters which impact size, charge, and drug encapsulation efficiency.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, intratumoral, and kidney dialytic infusion techniques.

In some embodiments, “particle count” or “population” refer to a group of particles including nanoparticles, including particles with uniform size, charge, shape, and composition, including nanoparticles with uniform size, charge, shape, and composition.

The term “pharmaceutical composition” refers to a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

“Pharmaceutically acceptable” means physiologically tolerable, for either human or veterinary application. Similarly, “pharmaceutical compositions” include formulations for human use and/or for veterinary use.

As used herein, the term “pharmaceutically acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality”means at least two.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” refers to non-naturally occurring peptides or polypeptides. Synthetic peptides or polypeptides can be synthesized, for example, using an automated polypeptide synthesizer. Various solid phase peptide synthesis methods are known to those of skill in the art.

As used herein, the term “prevent” means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

A “preventive” or “prophylactic” treatment is a treatment administered to a subject who does not exhibit signs, or exhibits only early signs, of a condition, disease, or disorder. A prophylactic or preventative treatment is administered for the purpose of decreasing the risk of developing pathology associated with developing the condition, disease, or disorder.

The term “protein” typically refers to large polypeptides (e.g., in some embodiments polypeptides of greater than 50 amino acids, in some embodiments polypeptides of greater than 100 amino acids, in some embodiments polypeptides of greater than 250 amino acids, in some embodiments polypeptides of greater than 500 amino acids, and in some embodiments polypeptides of greater than 1000 amino acids). Conventional notation is used herein to portray polypeptide sequences: the left-hand end of a polypeptide sequence is the amino-terminus; the right-hand end of a polypeptide sequence is the carboxyl-terminus.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process.

A “highly purified” compound as used herein refers to a compound that is in some embodiments greater than 90% pure, that is in some embodiments greater than 95% pure, and that is in some embodiments greater than 98% pure.

As used herein, the term “mammal” refers to any member of the class Mammalia, including, without limitation, humans and non-human primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Scalability” as used herein, describes the process of taking the nanoformulation from small scale to bulk to conduct in vitro and in vivo experiments to test the PK and PD effect of formulation.

As used herein, the phrase “sensitivity to histone deacetylase inhibitors” refers to a cell, tissue, or organ in which one or more undesirable histone deacetylase biological activities occur and/or have an effect that can be improved by treatment with a histone deacetylase inhibitor. In some embodiments, the undesirable histone deacetylase biological activity is associated with a disease, disorder, or condition that at least one symptom of which is improved and/or inhibited by treatment with a histone deacetylase inhibitor either alone or in combination with other treatments.

“Solvent” as used herein, refers in some embodiments to an organic substance used and, in some embodiments, acts as solvent for film-forming materials, romidepsin, and polymer. In some embodiments polar solvents, including water-miscible solvents, ethanol, dimethylsulfoxide (DMSO), acetone, tetrahydrofuran (THF), acetonitrile, or a combination of solvents are used to dissolve drug and polymer for preparation of nanoparticles. The physical properties of solvent influence the solubility of drug and polymer including the physio-chemical properties of NPs and overall nanoprecipitation method of making NPs. Acetonitrile is a particular example of solvent used to dissolve drug, polymer, and surfactant to prepare NPs by in a nanoprecipitation method. In some embodiments, acetonitrile (ACN) provided the greatest concentration, lowest PDI, and an average size. In addition to ACN, other solvents would be apparent to one of ordinary skill in the art upon a review of the instant disclosure.

“Anti-solvent” as used herein, refers an aqueous solvent, including but not limited to water or a buffer solution such as PBS, that can be employed to disperse solvent containing drug and polymer. Non-solvent and anti-solvent terms are interchangeable. In some embodiments, hydrophilic excipients can also be added to a non-solvent.

The term “subject” as used herein refers to a member of species for which treatment and/or prevention of a disease or disorder using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichthyes (bony fish), Amphibia (amphibians), Reptilia (reptiles), Aves (birds), and Mammalia (mammals), and all Orders and Families encompassed therein.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like. As such, the compositions and methods of the presently disclosed subject matter are appropriate for use in humans as well as in veterinary applications.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

A “subject” of analysis, diagnosis, or treatment is an animal. Such animals include mammals, in some embodiments, humans.

As used herein, a “subject in need thereof” is a patient, animal, mammal, or human, who will benefit from the compositions and/or methods of the presently disclosed subject matter.

The term “substantially pure” describes a compound, e.g., a protein or polypeptide, which has been separated from components which naturally accompany it. Typically, a compound is substantially pure when in some embodiments at least 10%, in some embodiments at least 20%, in some embodiments at least 50%, in some embodiments at least 60%, in some embodiments at least 75%, in some embodiments at least 90%, and in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

“Surfactant” is used herein as a material that supports NPs stability. In some embodiments, a surfactant is a compound that decreases the surface tension between two liquids, between a gas and a liquid, or between a liquid and a solid. In some embodiments, a surfactant composition includes but is not limited to a polaxamer (e.g., polaxamer 188, polaxamer 237, polaxamer 338, and polaxamer 407), Tween 20, Tween 80, polyvinyl alcohol (PVA), etc. In general, these materials are used as emulsifiers and can avoid aggregation.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

As used herein, the phrase “therapeutic agent” refers to an agent that is used to, for example, treat, inhibit, prevent, mitigate the effects of, reduce the severity of, reduce the likelihood of developing, slow the progression of, and/or cure, a disease or disorder.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

As used herein, the terms “vector”, “cloning vector”, and “expression vector” refer to a vehicle by which a polynucleotide sequence (e.g., a foreign gene) can be introduced into a host cell so as to transduce and/or transform the host cell in order to promote expression (e.g., transcription and translation) of the introduced sequence. Vectors include plasmids, phages, viruses, etc.

As used herein, the phrase “zeta potential” refers to a measurement of surface potential of a particle. In some embodiments of the presently disclosed subject matter, the particles have a zeta potential in the range of −25 to +25.

All genes, gene names, and gene products disclosed herein are intended to correspond to homologs and/or orthologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates.

### III. Exemplary Embodiments

In some embodiments, the presently disclosed subject matter relates to the compositions comprising detectable HDACi polymer nanoparticles and methods for using the same to diagnose and/or treat various diseases, disorders, and conditions associated with undesirable HDAC biological activities. An exemplary nanoparticle is a poly(D,L-lactide)-PEG-methyl ether (mPEG-PDLLA) nanoparticle. Some nanoparticles may employ or include other polymers, surfactants, lipids, or a combination of polymer and lipid. They may also employ other methods to produce core-shell, circular, sphere-shaped, spherical, micellar, mono, and bilayer polymersomes to compose one or more histone HDACis.

Thus, in some embodiments, the presently disclosed subject matter relates in some embodiments to compositions for use in preventing and/or treating a disease, disorder, and/or condition associated with sensitivity to HDAC inhibitors. In some embodiments, the presently disclosed subject matter relates to compositions comprising, consisting essentially of, or consisint of one or more histone deacetylase inhibitors (HDACi). Exemplary HDACi include vorinostat, romidepsin, belinostat, panobinostat, and chidamide.

In some embodiments, the HDACi is encapsulated in and/or otherwise associated with a nanoparticle. An exemplary nanoparticle is a poly(D,L-lactide)-PEG-methyl ether (mPEG-PDLLA) nanoparticle, although nanoparticles may employ or include other lipids, organic molecules, and/or inorganic molecules.

As such, in some embodiments the presently disclosed subject matter related to compositions comprising, consisting essentially of, or consisting of a histone deacetylase inhibitor (HDACi) encapsulated in and/or otherwise associated with a nanoparticle. In some embodiments, the HDACi is selected from the group consisting of vorinostat, romidepsin, belinostat, panobinostat, and chidamide, or any combination thereof, optionally wherein the HDACi is romidepsin. In some embodiments, the nanoparticle is a poly(D, L-lactide)-PEG-methyl ether (mPEG-PDLLA) nanoparticle. In some embodiments, a composition of the presently disclosed subject matter comprises one or more polymers and/or one or more surfactants. In some embodiments, the one or more polymers are selected from the group consisting of a polyester, optionally PDLLA and/or PLA, copolymers thereof, and blends thereof. In some embodiments, the polymer comprises a polymer selected from the group consisting of a synthetic polymer; a biodegradable polymer; a biocompatible polymer; an amphiphilic polymer; a diblock co-polymer; and blends thereof. In some embodiments, the polymer comprises a hydrophilic, PEG chain, optionally methoxy PEG, PEG-carboxylic acid, PEG-hydroxyl, and/or PEG amine as end cap and chain length range 2K-10K. In some embodiments, the polymer is a hydrophobic core-forming polymer, optionally a hydrophobic core-forming polymer selected from the group consisting of PDLLA and/or PLA. In some embodiments, the nanoparticle comprises a methyl ether-PDLLA (50:50). In some embodiments, one or more parameters of the composition selected from a group consisting of mode of phase addition, HDACi/polymer ratio, HDACi/surfactant ratio, solvent/anti-solvent ratio, rate of addition, and combinations thereof are optimized. In some embodiments, the HDACi/polymer ratio ranges from about 1:10 to about 1:100 W/W, optionally 1:10 to about 1:50 W/W; the HDACi/surfactant ratio ranges from about 1:0.05 to about 1:0.2 W/W; the solvent/anti-solvent ratio ranges from about 1:10 to about 1:1, optionally wherein the anti-solvent is selected from the group consisting of water, PBS, or another ionic buffer solution; and/or the rate of addition ranges from about 10 to about 500 mL/hour, optionally about 10 to about 50 mL/hour.

The nanoparticles of the presently disclosed subject matter include one or more detectable agents. As used herein, the phrase “detectable agent” refers to any moiety that, when complexed to and/or otherwise associated with the presently disclosed nanoparticles, renders the nanoparticles detectable in vitro, ex vivo, and/or in vivo using any methodology. Exempalry detection methodologies include but are not limited to ultrasound, magnetic resonance imaging, positron emission tomography, computed tomography, and photoacoustic imaging.

### III. A. Formulations

The compositions (e.g., HDAC NPs) of the presently disclosed subject matter can be administered in any formulation or route that would be expected to deliver the compositions to whatever target site might be appropriate.

The compositions of the presently disclosed subject matter comprise in some embodiments a composition that includes a carrier, particularly a pharmaceutically acceptable carrier, such as but not limited to a carrier pharmaceutically acceptable in humans. Any suitable pharmaceutical formulation can be used to prepare the compositions for administration to a subject.

For example, suitable formulations can include aqueous and non-aqueous sterile injection solutions that can contain anti-oxidants, buffers, bacteriostatics, bactericidal antibiotics, and solutes that render the formulation isotonic with the bodily fluids of the intended recipient.

It should be understood that in addition to the ingredients particularly mentioned above the formulations of the presently disclosed subject matter can include other agents conventional in the art with regard to the type of formulation in question. For example, sterile pyrogen-free aqueous and non-aqueous solutions can be used.

The therapeutic regimens and compositions of the presently disclosed subject matter can be used with additional adjuvants or biological response modifiers including, but not limited to, cytokines and other immunomodulating compounds.

### III. B. Administration

Suitable methods for administration of the compositions of the presently disclosed subject matter include, but are not limited to intravenous administration and delivery directly to the target tissue or organ (e.g., a tumor, a cancer, or endothelial tissue associated therewith). Exemplary routes of administration include parenteral, enteral, intravenous, intraarterial, intracardiac, intrapericardial, intraosseal, intracutaneous, subcutaneous, intradermal, subdermal, transdermal, intrathecal, intramuscular, intraperitoneal, intrasternal, parenchymatous, oral, sublingual, buccal, inhalational, and intranasal. The selection of a particular route of administration can be made based at least in part on the nature of the formulation and the ultimate target site where the compositions of the presently disclosed subject matter are desired to act. In some embodiments, the method of administration encompasses features for regionalized delivery or accumulation of the compositions at the site in need of treatment. In some embodiments, the compositions are delivered directly into the site to be treated.

### III. C. Doses

An effective dose of a composition of the presently disclosed subject matter is administered to a subject in need thereof. A “treatment effective amount” or a “therapeutic amount” is an amount of a therapeutic composition sufficient to produce a measurable response (e.g., a biologically or clinically relevant response in a subject being treated, such as but not limited to a reduction in the growth and/or proliferation of a tumor and/or a cancer, and/or a reduction in the extent to and/or timing at which a disease, disorder, and/or condition develops in a subject.). Actual dosage levels of active ingredients in the compositions of the presently disclosed subject matter can be varied so as to administer an amount of the active compound(s) that is effective to achieve the desired therapeutic response for a particular subject. The selected dosage level will depend upon the activity of the composition, the route of administration, combination with other drugs or treatments, the severity of the disease, disorder, and/or condition being treated, and the condition and prior medical history of the subject being treated. However, it is within the skill of the art to start doses of the compositions of the presently disclosed subject matter at levels lower than required to achieve the desired therapeutic effect and to gradually increase the dosage until the desired effect is achieved. The potency of a composition can vary, and therefore a “treatment effective amount” can vary. However, using the methods described herein, one skilled in the art can readily assess the potency and efficacy of a composition of the presently disclosed subject matter and adjust the therapeutic regimen accordingly.

After review of the disclosure of the presently disclosed subject matter presented herein, one of ordinary skill in the art can tailor the dosages to an individual subject, taking into account the particular formulation, method of administration to be used with the composition, and particular disease, disorder, and/or condition treated. Further calculations of dose can consider subject height and weight, severity and stage of symptoms, and the presence of additional deleterious physical conditions. Such adjustments or variations, as well as evaluation of when and how to make such adjustments or variations, are well known to those of ordinary skill in the art of medicine.

In some embodiments, the presently disclosed subject matter also relates to methods for treating a disease, disorder, or condition associated with sensitivity to histone deacetylase inhibitors, the method comprising administering to a subject in need thereof an effective amount of a composition as disclosed herein. In some embodiments, the disease, disorder, or condition associated with sensitivity to histone deacetylase inhibitors is a tumor and/or a cancer. In some embodiments, the tumor and/or the cancer is selected from the group consisting of cutaneous T cell lymphoma (CTCL), peripheral T cell lymphoma (PTCL), and multiple myeloma. In some embodiments, the disease, disorder, or condition associated with sensitivity to histone deacetylase inhibitors is an autoimmune disease, disorder, or condition, which in some embodiments can be large granular lymphocytic leukemia.

As such, the presently disclosed subject matter also relates in some embodiments to methods for treating diseases, disorders, and/or conditions associated with sensitivity to histone deacetylase inhibitors. In some embodiments, the methods comprise, consist essentially of, or consist of administering to a subject in need thereof an effective amount of a composition comprising, consisting essentially of, or consisting of a histone deacetylase inhibitor (HDACi) encapsulated in and/or otherwise associated with a nanoparticle. In some embodiments, the disease, disorder, and/or condition associated with senstivity to histone deacetylase inhibitors is a tumor and/or a cancer. In some embodiments, the tumor and/or the cancer is selected from the group consisting of cutaneous T cell lymphoma (CTCL), peripheral T cell lymphoma (PTCL), multiple myeloma, large granular lymphocytic leukemia (LGLL), and adult T cell leukemia/lymphoma.

The presently disclosed subject matter also relates in some embodiments to methods for treating diseases, disorders, and/or conditions associated with sensitivity to a histone deacetylase inhibitor (HDACi) by administering to a subject in need thereof an effective amount of a composition as disclosed herein. In some embodiments, the disease, disorder, or condition associated with sensitivity to an HDACi is a tumor and/or a cancer, an inflammatory disease, disorder, or condition; an autimmune disease, disorder, or condition; or any combination thereof. In some embodiments, the tumor and/or the cancer is selected from the group consisting of cutaneous T cell lymphoma (CTCL), peripheral T cell lymphoma (PTCL), multiple myeloma, large granular lymphocytic leukemia (LGLL), and adult T cell leukemia/lymphoma.

In some embodiments, the presently disclosed subject matter also relates to methods for inhibiting the growth, proliferation, and/or metastasis of a tumor and/or a cancer associated with sensitivity to histone deacetylase inhibitors. In some embodiments, the methods comprise, consist essentially of, or consist of administering to a subject in need thereof an effective amount of a composition as disclosed herein. In some embodiments, the tumor and/or the cancer is selected from the group consisting of cutaneous T cell lymphoma (CTCL), peripheral T cell lymphoma (PTCL), and multiple myeloma.

As such, the presently disclosed subject matter also relates in some embodiments to methods for inhibiting the growth, proliferation, and/or metastasis of a tumor and/or a cancer associated with sensitivity to histone deacetylase inhibitors. In some embodiments, the methods comprise, consist essentially of, or consist of administering to a subject in need thereof an effective amount of a composition comprising, consisting essentially of, or consisting of a histone deacetylase inhibitor (HDACi) encapsulated in and/or otherwise associated with a nanoparticle. In some embodiments, the tumor and/or the cancer is selected from the group consisting of cutaneous T cell lymphoma (CTCL), peripheral T cell lymphoma (PTCL), and multiple myeloma. In some embodiments, the presently disclosed methods further comprise, consist essentially of, or consist of administering to the subject at least one additional therapeutically active agent. In some embodiments, the at least one additional therapeutically active agent is a chemotherapeutic agent. In some embodiments, the chemotherapeutic agent is selected from the group consisting of acitretin, bexarotene, an interferon, optionally an alpha and/or a gamma interferon, cyclosporine A, methotrexate, romidepsin, vorinostat, cyclophosphamide, doxorubicin, vincristine, prednisone, dexamethasone, etoposide, vincristine, brentuximab vedotin, a histone lysine methyltransferase (HMT) enhancer of zeste homolog 1 (EZH1) inhibitor, a histone lysine methyltransferase (HMT) enhancer of zeste homolog 2 (EZH2) inhibitor, a JAK/STAT pathway inhibitor, optionally ruxolitinib, tofacitinib, baricitinib, fedratinib, upadacitinib, abrocitinib, and/or deucravacitinib; a hypomethylating agent, an immunotherapeutic agent, optionally a monoclonal antibody, further optionally alemtuzumab; ranimustine, vindesine, carboplatin, and combinations thereof. In some embodiments, the method comprises a combination therapy comprising a combination of active agents selected from the group consisting of cyclophosphamide/doxorubicin/vincristine/prednisone (CHOP); etoposide/vincristine/doxorubicin/cyclophosphamide/prednisone (CHOEP); cyclophosphamide/doxorubicin/prednisone/brentuximab vedotin (CHP-BV); brentuximab vedotin/cyclophosphamide/doxorubicin/prednisone (CHP); and vincristine/cyclophosphamide/doxorubicin/prednisone (VCAP) plus doxorubicin/ranimustine/prednisone (AMP), and vindesine/etoposide/carboplatin/prednisone (VECP; VCAP-AMP-VECP).

The presently disclosed subject matter also relates in some embodiments to methods for inhibiting the growth, proliferation, and/or metastasis of a tumor and/or a cancer associated with sensitivity to a histone deacetylase inhibitor (HDACi). In some embodiments, the methods comprise, consist essentially of, or consist of administering to a subject in need thereof an effective amount of a composition as disclosed herein. In some embodiments, the tumor and/or the cancer is selected from the group consisting of cutaneous T cell lymphoma (CTCL), peripheral T cell lymphoma (PTCL), multiple myeloma, large granular lymphocytic leukemia (LGLL), and adult T cell leukemia/lymphoma.

In some embodiments, the presently disclosed methods further comprise, consist essentially of, or consist of administering to the subject at least one additional therapeutically active agent. In some embodiments, the at least one additional therapeutically active agent is a chemotherapeutic agent. In some embodiments, the chemotherapeutic agent is selected from the group consisting of acitretin, bexarotene, an interferon, optionally an alpha and/or a gamma interferon, cyclosporine A, methotrexate, romidepsin, vorinostat, cyclophosphamide, doxorubicin, vincristine, prednisone, dexamethasone, etoposide, vincristine, brentuximab vedotin, a histone lysine methyltransferase (HMT) enhancer of zeste homolog 1 (EZH1) inhibitor, a histone lysine methyltransferase (HMT) enhancer of zeste homolog 2 (EZH2) inhibitor, a JAK/STAT pathway inhibitor, optionally ruxolitinib, tofacitinib, baricitinib, fedratinib, upadacitinib, abrocitinib, and/or deucravacitinib; a hypomethylating agent, an immunotherapeutic agent, optionally a monoclonal antibody, further optionally alemtuzumab; ranimustine, vindesine, carboplatin, and combinations thereof. In some embodiments, the chemotherapeutic agent is selected from the group consisting of acitretin, bexarotene, an interferon, optionally an alpha and/or a gamma interferon, cyclosporine A, methotrexate, romidepsin, vorinostat, cyclophosphamide, doxorubicin, vincristine, prednisone, dexamethasone, etoposide, vincristine, brentuximab vedotin, a histone lysine methyltransferase (HMT) enhancer of zeste homolog 1 (EZH1) inhibitor, a histone lysine methyltransferase (HMT) enhancer of zeste homolog 2 (EZH2) inhibitor, a JAK/STAT pathway inhibitor, optionally ruxolitinib, tofacitinib, baricitinib, fedratinib, upadacitinib, abrocitinib, and/or deucravacitinib; a hypomethylating agent, an immunotherapeutic agent, optionally a monoclonal antibody, further optionally alemtuzumab; ranimustine, vindesine, carboplatin, and combinations thereof.

In some embodiments, the presently disclosed methods are part of a combination therapy. In some embodiments, the combination therapy comprises administration of a combination of active agents, wherein the combination of active agents are selected from the group consisting of cyclophosphamide/doxorubicin/vincristine/prednisone (CHOP); etoposide/vincristine/doxorubicin/cyclophosphamide/prednisone (CHOEP); cyclophosphamide/doxorubicin/prednisone/brentuximab vedotin (CHP-BV); brentuximab vedotin/cyclophosphamide/doxorubicin/prednisone (CHP); and vincristine/cyclophosphamide/doxorubicin/prednisone (VCAP) plus doxorubicin/ranimustine/prednisone (AMP), and vindesine/etoposide/carboplatin/prednisone (VECP; VCAP-AMP-VECP).

### III. D. Combination Therapies

In some embodiments, the presently disclosed subject matter relates to combination therapies in which a given disease, disorder, or condition associated with sensitivity to histone deacetylase inhibitors is treated with an HDAC inhibitor and also one or more additional therapeutic agents that are appropriate for the disease, disorder, or condition to be treated. Thus, in some embodiments, the presently disclosed methods can further comprise, consist essentially of, or consist of administering to the subject at least one additional therapeutically active agent. In some embodiments wherein the disease, disorder, or condition to be treated is a tumor and/or a cancer, the at least one additional therapeutically active agent can be a chemotherapeutic agent. Chemotherapeutic (cytotoxic) agents including, but are not limited to, 5-fluorouracil, bleomycin, busulfan, camptothecins, carboplatin, chlorambucil, cisplatin (CDDP), cyclophosphamide, dactinomycin, daunorubicin, doxorubicin, estrogen receptor binding agents, etoposide (VP16), farnesyl-protein transferase inhibitors, gemcitabine, ifosfamide, mechlorethamine, melphalan, mitomycin, navelbine, nitrosurea, plicomycin, procarbazine, raioxifene, tamoxifen, taxol, temazolomide (an aqueous form of DTIC), transplatinum, vinblastine, methotrexate, vincristine, and any analogs and/or derivatives or variants of the foregoing. Most chemotherapeutic agents fall into the categories of alkylating agents, antimetabolites, antitumor antibiotics, corticosteroid hormones, mitotic inhibitors, and nitrosoureas, hormone agents, miscellaneous agents, and any analog, derivative, or variant thereof. In some embodiments, the chemotherapeutic agent is selected from the group consisting of acitretin, bexarotene, an interferon, optionally an alpha and/or a gamma interferon, cyclosporine A, methotrexate, romidepsin, vorinostat, cyclophosphamide, doxorubicin, vincristine, prednisone, dexamethasone, etoposide, vincristine, brentuximab vedotin, a histone lysine methyltransferase (HMT) enhancer of zeste homolog 1 (EZH1) inhibitor, a histone lysine methyltransferase (HMT) enhancer of zeste homolog 2 (EZH2) inhibitor, a JAK/STAT pathway inhibitor, optionally ruxolitinib, tofacitinib, baricitinib, fedratinib, upadacitinib, abrocitinib, and/or deucravacitinib; a hypomethylating agent, an immunotherapeutic agent, optionally a monoclonal antibody, further optionally alemtuzumab; ranimustine, vindesine, carboplatin, and combinations thereof. In some embodiments, the at least one additional therapeutically active agent is cyclosporine A (CSA), a hypomethylating agent, cladribine, or pralatrexate. In some embodiments, the at least one additional therapeutically active agent is an active agent that targets PI3K, Bcl-2, BTK, HDAC, DNMT, BRAF, or MEK, and/or is an active agent that is classified as targeting epigenetic phenomena, and/or is an immunologic therapeutic such as but not limited to a monoclonal antibody, an antibody/drug conjugate, a bispecific antibody, and/or an adoptive cellular therapy such as but not limited to a CAR-T cell or CAR-T-based therapeutic including but not limited to commercially available T cell therapeutics.

In some embodiments, a combination therapy of the presently disclosed subject matter relates to methods comprising administration of a combination of active agents, wherein the combination of active agents are in some embodiments selected from the group consisting of cyclophosphamide/doxorubicin/vincristine/prednisone (CHOP); etoposide/vincristine/doxorubicin/cyclophosphamide/prednisone (CHOEP); cyclophosphamide/doxorubicin/prednisone/brentuximab vedotin (CHP-BV); brentuximab vedotin/cyclophosphamide/doxorubicin/prednisone (CHP); and vincristine/cyclophosphamide/doxorubicin/prednisone (VCAP) plus doxorubicin/ranimustine/prednisone (AMP), and vindesine/etoposide/carboplatin/prednisone (VECP; VCAP-AMP-VECP).

In some embodiments, the tumor and/or the cancer is sensitive to and/or refractory, relapsed, and/or resistant to one or more chemotherapeutic agents such as, but not limited to a platinum-based agent, a taxane, an alkylating agent, an anthracycline (e.g., doxorubicin including but not limited to liposomal doxorubicin), an antimetabolite, and/or a vinca alkaloid. In some embodiments, the cancer is an ovarian cancer, and the ovarian cancer is refractory, relapsed, or resistant to a platinum-based agent (e.g., carboplatin, cisplatin, oxaliplatin), a taxane (e.g., paclitaxel, docetaxel, larotaxel, cabazitaxel), and/or an anthracycline (e.g., doxorubicin including but not limited to liposomal doxorubicin). In some embodiments, the cancer is colorectal cancer, and the cancer is refractory, relapsed, or resistant to an antimetabolite (e.g., an antifolate (e.g., pemetrexed, floxuridine, raltitrexed) a pyrimidine analogue (e.g., capecitabine, cytrarabine, gemcitabine, 5FU)), and/or a platinum-based agent (e.g., carboplatin, cisplatin, oxaliplatin). In some embodiments, the cancer is lung cancer, and the cancer is refractory, relapsed, or resistant to a taxane (e.g., paclitaxel, docetaxel, larotaxel, cabazitaxel), a platinum-based agent (e.g., carboplatin, cisplatin, oxaliplatin), a vinca alkaloid (e.g., vinblastine, vincristine, vindesine, vinorelbine), a vascular endothelial growth factor (VEGF) pathway inhibitor, an epidermal growth factor (EGF) pathway inhibitor) and/or an antimetabolite (e.g., an antifolate including but not limited to pemetrexed, floxuridine, or raltitrexed), and a pyrimidine analogue (e.g., capecitabine, cytrarabine, gemcitabine, 5FU). In some embodiments, the cancer is breast cancer, and the cancer is refractory, relapsed, or resistant to a taxane (e.g., paclitaxel, docetaxel, larotaxel, cabazitaxel), a VEGF pathway inhibitor, an anthracycline (e.g., daunorubicin, doxorubicin including but not limited to liposomal doxorubicin, epirubicin, valrubicin, idarubicin), a platinum-based agent (e.g., carboplatin, cisplatin, oxaliplatin), and/or an antimetabolite (e.g., an antifolate including but not limited to pemetrexed, floxuridine, or raltitrexed), and a pyrimidine analogue (e.g., capecitabine, cytrarabine, gemcitabine, 5FU). In some embodiments, the cancer is gastric cancer, and the cancer is refractory, relapsed, or resistant to an antimetabolite (e.g., an antifolate including but not limited to pemetrexed, floxuridine, raltitrexed) and a pyrimidine analogue (e.g., capecitabine, cytrarabine, gemcitabine, 5FU) and/or a platinum-based agent (e.g., carboplatin, cisplatin, oxaliplatin). In some embodiments, the provision of the HDACi as part of a nanoparticle overcomes the tumor's and/or the cancer's nature as being refractory, relapsed, and/or resistant to one or more chemotherapeutic agents. Alternatively or in addition, the compositions and methods of the presently disclosed subject matter can include active agents that are routinely used in the treatment of, for example, lymphoma including but not limited to alkylating agents (e.g., cyclophosphamide, ifosphamide dacarbazine, and BCNU), anthracyclines, vinca alkaloids, platinum analogs, antimetabolites (e.g., methotrexate, Ara-C, gemcitabine), topoisomerase inhibitors, steroids, and combinations thereof.

In some embodiments wherein the disease, disorder, or condition to be treated is an inflammatory disease, disorder, or condition. Exemplary, non-limiting inflammatory diseases, disorders, or conditions include Fatty liver disease, endometriosis, types 1 and 2 diabetes, inflammatory bowel disease, asthma, obesity, Alzheimer's and Parkinson's diseases, Ankylosing Spondylitis (AS), Antiphospholipid Antibody Syndrome (APS), Gout, Inflammatory Arthritis Center, Myositis, Rheumatoid Arthritis, Scleroderma, Sjogren's Syndrome, Systemic Lupus Erythematosus (SLE, Lupus), Vasculitis, and tumors/cancers. In some embodiments, the at least one additional therapeutically active agent can thus be any anti-inflammatory agent typically employed in the treatment/management of any of these diseases, disorders, or conditions. Exemplary anti-inflammatory agents include, but are not limited to Alclofenac; Alclometasone Dipropionate; Algestone Acetonide; Alpha Amylase; Amcinafal; Amcinafide; Amfenac Sodium; Amiprilose Hydrochloride; Anakinra; Anirolac; Anitrazafen; Apazone; Balsalazide Disodium; Bendazac; Benoxaprofen; Benzydamine Hydrochloride; Bromelains; Broperamole; Budesonide; Carprofen; Cicloprofen; Cintazone; Cliprofen; Clobetasol Propionate; Clobetasone Butyrate; Clopirac; Cloticasone Propionate; Cormethasone Acetate; Cortodoxone; Deflazacort; Desonide; Desoximetasone; Dexamethasone Dipropionate; Diclofenac Potassium; Diclofenac Sodium; Diflorasone Diacetate; Diflumidone Sodium; Diflunisal; Difluprednate; Diftalone; Dimethyl Sulfoxide; Drocinonide; Endrysone; Enlimomab; Enolicam Sodium; Epirizole; Etodolac; Etofenamate; Felbinac; Fenamole; Fenbufen; Fenclofenac; Fenclorac; Fendosal; Fenpipalone; Fentiazac; Flazalone; Fluazacort; Flufenamic Acid; Flumizole; Flunisolide Acetate; Flunixin; Flunixin Meglumine; Fluocortin Butyl; Fluorometholone Acetate; Fluquazone; Flurbiprofen; Fluretofen; Fluticasone Propionate; Furaprofen; Furobufen; Halcinonide; Halobetasol Propionate; Halopredone Acetate; Ibufenac; Ibuprofen; Ibuprofen Aluminum; Ibuprofen Piconol; Ilonidap; Indomethacin; Indomethacin Sodium; Indoprofen; Indoxole; Intrazole; Isoflupredone Acetate; Isoxepac; Isoxicam; Ketoprofen; Lofemizole Hydrochloride; Lornoxicam; Loteprednol Etabonate; Meclofenamate Sodium; Meclofenamic Acid; Meclorisone Dibutyrate; Mefenamic Acid; Mesalamine; Meseclazone; Methylprednisolone Suleptanate; Momiflumate; Nabumetone; Naproxen; Naproxen Sodium; Naproxol; Nimazone; Olsalazine Sodium; Orgotein; Orpanoxin; Oxaprozin; Oxyphenbutazone; Paranyline Hydrochloride; Pentosan Polysulfate Sodium; Phenbutazone Sodium Glycerate; Pirfenidone; Piroxicam; Piroxicam Cinnamate; Piroxicam Olamine; Pirprofen; Prednazate; Prifelone; Prodolic Acid; Proquazone; Proxazole; Proxazole Citrate; Rimexolone; Romazarit; Salcolex; Salnacedin; Salsalate; Sanguinarium Chloride; Seclazone; Sermetacin; Sudoxicam; Sulindac; Suprofen; Talmetacin; Talniflumate; Talosalate; Tebufelone; Tenidap; Tenidap Sodium; Tenoxicam; Tesicam; Tesimide; Tetrydamine; Tiopinac; Tixocortol Pivalate; Tolmetin; Tolmetin Sodium; Triclonide; Triflumidate; Zidometacin; Zomepirac Sodium; methotrexate, dexamethasone, dexamethasone alcohol, dexamethasone sodium phosphate, fluromethalone acetate, fluromethalone alcohol, lotoprendol etabonate, medrysone, prednisolone acetate, prednisolone sodium phosphate, difluprednate, rimexolone, hydrocortisone, hydrocortisone acetate, lodoxamide tromethamine, glucocorticoids, diclofenac, and any combination thereof.

In some embodiments wherein the disease, disorder, or condition to be treated is an autoimmune disease, disorder, or condition. Exemplary autoimmune diseases, disorders, or conditions include Addison's disease, Celiac disease-sprue (gluten-sensitive enteropathy), dermatomyositis, Grave's disease, Hashimoto's thyroiditis, Multiple sclerosis, Myasthenia gravis, Pernicious anemia, Reactive arthritis, Rheumatoid arthritis, Psoriasis/psoriatic arthritis, multiple sclerosis, Sjögren's syndrome, Systemic lupus erythematosus (SLE), type 1 diabetes, Inflammatory bowel disease (including Crohn's disease and ulcerative colitis), autoimmune vasculitis, Guillain-Barre syndrome, and Chronic inflammatory demyelinating polyneuropathy. In some embodiments, the at least one additional therapeutically active agent can thus be any therapeutic agent typically employed in the treatment/management of any of these diseases, disorders, or conditions, including but not limited to the anti-inflammatoires listed above and/or steroids (including but not limited to prednisone, methylprednisolone, and dexamethasone), colchicine, hydroxychloroquine (Plaquenil), Sulfasalazine, dapsone, methotrexate, Mycophenolate Mofetil (Cellcept, Myfortic), Azathioprine (Imuran), anti-IL-1 biologics including but not limited to Anakinra/Kineret, Canakinumab/Ilaris, and Rilonacept/Arcalyst), anti-TNF biologics (including but not limited to nfliximab/Remicade, Adalimumab/Humira, Golimumab/Simponi, Etanercept/Enbrel, and Certolizumab/Cimzia), anti-IL-6 biologics (including but not limited to Tocilizumab/Actemra and Sarilumab/Kevzara), complement inhibitors (including but not limited to Eculizumab), anti-CD20 biologics (including but not limited to Rituximab/Rituxan), B cell growth factor targeting biologics (including but not limited to Belimumab/Benlysta), therapeutics targetted to adaptive immunity-T cells (including but not limited to cyclosporine), therapeutics targetted to T cell co-stimulation and/or activation (including but not limited to Abatacept/Orencia), anti-IL-17 biologics (including but not limited to Secukinumab/Cosentyx), Ixekizumab/Taltz), Brodalumab/Siliq), anti-IL-23 biologics (including but not limited to Guselkumab/Tremfya), anti-IL-12/23 biologics (including but not limited to Ustekinumab/Stelara), anti-IL-5 biologics (including but not limited to Mepolizumab/Nucala, Reslizumab/Cinqair, Benralizumab/Fasenra), anti-IL-4/IL-23 biologics (including but not limited to Dupilumab/Dupixent), biologics targeting IgE (including but not limited to Omalizumab/Xolair), agents targeting lymphocyte movement/trafficking (including but not limited to Vedolizumab/Entyvio), small molecule inhibitors of any biological activity that is associated with any autoimmune disease, disorder, or condition, including but not limited to JAK inhibitors (such as but not limited to Tofacitinib/Xeljanz, Upadacitinib/Rinvoq, and Baricitinib/Olumiant), or any combination of any of these agentsor any pharmaceutically acceptable salt or derivative thereof. See U.S. Pat. No. 10,092,584, the entire disclosure of which is incorporated herein by reference.

As such, the presently disclosed subject matter also relates in some embodiments to methods for treating an inflammatory and/or an autoimmune disease, disorder, or condition. In some embodiments, the inflammatory and/or an autoimmune disease, disorder, or condition is selected from the group consisting of fatty liver disease, endometriosis, types 1 and 2 diabetes, inflammatory bowel disease, asthma, obesity, Alzheimer's and Parkinson's diseases, Ankylosing Spondylitis (AS), Antiphospholipid Antibody Syndrome (APS), Gout, Inflammatory Arthritis Center, Myositis, Rheumatoid Arthritis, Scleroderma, Sjogren's Syndrome, Systemic Lupus Erythematosus (SLE, Lupus), vasculitis, Addison's disease, Celiac disease-sprue (gluten-sensitive enteropathy), dermatomyositis, Grave's disease, Hashimoto's thyroiditis, Multiple sclerosis, Myasthenia gravis, Pernicious anemia, Reactive arthritis, Rheumatoid arthritis, Psoriasis/psoriatic arthritis, multiple sclerosis, Sjögren's syndrome, Systemic lupus erythematosus (SLE), type 1 diabetes, Inflammatory bowel disease (including Crohn's disease and ulcerative colitis), autoimmune vasculitis, Guillain-Barre syndrome, and Chronic inflammatory demyelinating polyneuropathy. In some embodiments, the presently disclosed methods comprise administering to a subject in need thereof a composition of the presently disclosed subject matter in combination with at least one additional therapeutically active agent. In some embodiments, the at least one additional therapeutically active agent is an anti-inflammatory and/or an immunosuppresant agent.

The presently disclosed subject matter also relates in some embodiments to methods for treating inflammatory and/or autoimmune diseases, disorders, and/or conditions associated with sensitivity to a histone deacetylase inhibitor (HDACi). In some embodiments, the method comprises, consists essentially of, or consists of administering to a subject in need thereof an effective amount of a composition as disclosed herein. In some embodiments, the inflammatory and/or an autoimmune disease, disorder, or condition is selected from the group consisting of fatty liver disease, endometriosis, types 1 and 2 diabetes, inflammatory bowel disease, asthma, obesity, Alzheimer's and Parkinson's diseases, Ankylosing Spondylitis (AS), Antiphospholipid Antibody Syndrome (APS), Gout, Inflammatory Arthritis Center, Myositis, Rheumatoid Arthritis, Scleroderma, Sjogren's Syndrome, Systemic Lupus Erythematosus (SLE, Lupus), vasculitis, Addison's disease, Celiac disease-sprue (gluten-sensitive enteropathy), dermatomyositis, Grave's disease, Hashimoto's thyroiditis, Multiple sclerosis, Myasthenia gravis, Pernicious anemia, Reactive arthritis, Psoriasis/psoriatic arthritis, multiple sclerosis, Systemic lupus erythematosus (SLE), type 1 diabetes, Inflammatory bowel disease (including Crohn's disease and ulcerative colitis), autoimmune vasculitis, Guillain-Barre syndrome, and Chronic inflammatory demyelinating polyneuropathy.

In some embodiments, the presently disclosed methods further comprise, consist essentially of, or consist of administering to the subject at least one additional therapeutically active agent. In some embodiments, the at least one additional therapeutically active agent is an anti-inflammatory and/or an immunosuppresant agent.

### III. E. Nanoparticle Synthesis

In some embodiments, the presently disclosed subject matter provides methods for synthesizing nanoparticles, the useful approach in the methods, and the nanoparticles (NPs) made by the method and the composition thereof. In some embodiments, the presently disclosed subject matter relates to the synthesis of a nanoparticle comprising a histone deacetylase inhibitor, in some embodiments, romidepsin. In some embodiments, the method described herein involves utilizing a multi-channel derived controlled addition of a phase to a phase in a “high throughput parallel manner” to optimize NP properties on a multi-point stirrer. The particles are formed at the interface of the two solutions. In some embodiments, the method is semiautomatic and generates monodispersed NPs with well-defined morphology. The method herein involves mixing of one or more materials that form nanoparticles with a second solution or anti-solvent. The population of particles produced using the methods have a range of uniform sizes and shapes. The particles produced in accordance with some embodiments of the presently disclosed method can achieve relatively precise desired concentrations of romidepsin. In some embodiments, the produced particles have negative zeta potential. In some embodiments, the method created herein is an iterative, rapidly optimizing, low-cost fabrication technique to generate stable and scalable formulations. In some embodiments, the method describes highly effective reproducible scale-up synthesis.

As such, the presently disclosed subject matter also relates in some embodiments to methods for fabricating nanoparticles comprising one or more drug molecules. In some embodiments, the methods comprise, consist essentially of, or consist of: (a) varying in one or more iterations two or more parameters of a first or subsequent reaction mixture comprising a drug molecule and one or more polymers; (b) selecting a desired combination of parameters for a further reaction mixture based on the varying of step (a); and (c) precipitating a nanoparticle comprising the drug molecule from the further reaction mixture. In some embodiments, the reaction mixture further comprises a reaction mixture selected from the group consisting of a solvent, a non-solvent, a surfactant, and combinations thereof. In some embodiments, the solvent is an organic solvent. In some embodiments, the non-solvent is an aqueous solvent, water, or PBS buffer.

In some embodiments, the particles are prepared from polymers. The polymeric materials can be biocompatible and biodegradable. In some embodiments, the composition comprising a methoxy poly (ethylene glycol)-b-poly(D, L-Lactide), methoxy poly(ethylene glycol)-b-poly(lactic-co-glycolic acid) di-block co-polymers. In some embodiments, the population of produced particles have a monomethyl polyethylene glycol (methoxy PEG, or mPEG). In some embodiments, the core forming hydrophobic polymer chain lengths and molecular weights are variable. In some embodiments, the shell forming hydrophilic polyethylene glycol ratios are variable and controlled. It is often considered that the formulation containing polyethylene glycol protects the particles from aggregation and “opsonization”. It is often desirable to produce biocompatible particles with “stealth” properties. In some embodiments, a stealth property involves evading the immune system, metabolic routes of clearance, and the like. In some embodiments, the nanoparticle composition produced using this (these) method(s) is stable and comprises a nonionic surfactant, such as poloxamer 188. The compositions may have properties which facilitate improved pharmacologic disposition and pharmacokinetic features, toxicity and efficacy. In some embodiments, the presently disclosed subject matter provides a detailed method for the synthesis and optimization of a romidepsin polymer nanoparticle formulation for therapeutic use.

To address challenges associated with the established physio-chemical properties and toxicities associated with romidepsin, the presently disclosed subject matter provides in some embodiments an optimized nanotechnology approach to synthesize versions of the drug with improved pharmacologic behavior. These approaches can allow for a highly loading and release of the drug in an efficient prescribed manner. The unique properties of nanoparticles, such as their small size, large surface-to-volume ratios, the ability to create rational combinatorial nanotherapeutics, and the ability to bioconjugate honing motifs on their surface, provides many advantages over traditional small molecule drug design and discovery.

Based on the properties of nanoparticles and the methodology described herein, a nanoparticle comprising romidepsin (embodiments of which are referred to herein as “NanoRomidepsin” and “NanoRomi”) produces a nanotherapeutic with substantially improved drug properties, rendering it a more advantageous drug for the treatment of disease. The presently disclosed subject matter relates in some embodiments to novel polymer nanoparticle formulated with romidepsin. The formulation of the nanoparticle has been engineered to facilitate the expected desired size, encapsulation efficiency, and pharmacokinetic parameters. One or more of the listed parameters, including some or all of the listed parameters, can play a role in influencing the nanoparticle's stability, size, polydispersity index (PDI), zeta potential, and encapsulation of drug. Exemplary parameters can include one or more of the following: PDI in a range of 0 to about 0.3, size in the range of about 30-150 nanometers, a morphology selected from the group consisting of spherical, rod, and cylindrical, optionally spherical; a zeta potential in the range of about −30 to about +30, about 50-60% encapsulation efficiency; a concentration of about 500 to about 600μg/mL; use of a PEG in a range of about 2K to about 10K, and a polymer size of about 4K to about 25K. The factors, including but not limited to the chemical nature of core-forming block polymer, molecular mass of hydrophilic block polymer, concentration of the polymer, controlled addition of solvent, ratio of solvent to anti-solvent, pH of anti-solvent, choice and percent concentration of surfactant, and the core shell nature of particles have a role on the physio-chemical properties of the nanoparticle. The presently disclosed nanoparticles have been shown to improve the pharmacokinetic features of the drug.

In some embodiments, the presently disclosed methods comprise optimization of one or more parameters selected from a group consisting of mode of phase addition, a drug/polymer ratio, a drug/surfactant ratio, solvent/anti-solvent ratio, rate of addition, and combinations thereof. In some embodiments, the drug is HDACi, optionally romidepsin. In some embodiments, the HDACi/polymer ratio ranges from about 1:10 to about 1:100 W/W, optionally 1:10 to about 1:50 W/W; the HDACi/surfactant ratio ranges from about 1:0.05 to about 1:0.2 W/W; the solvent/anti-solvent ratio ranges from about 1:10 to about 1:1, optionally wherein the anti-solvent is selected from the group consisting of water, PBS, or another ionic buffer solution; and/or the rate of addition ranges from about 10 to about 500 mL/hour, optionally about 10 to about 50 mL/hour.

An exemplary method for preparing the compositions of the presently disclosed subject matter is disclosed in PCT International Patent Application Publication No. WO 2023/06463, which is incorporated herein by reference in its entirety.

### EXAMPLES

The following EXAMPLES provide illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

Without further description, it is believed that one of ordinary skill in the art can, using the preceding description and the following illustrative EXAMPLES, make and utilize the compounds of the presently disclosed subject matter and practice the methods of the presently disclosed subject matter. The following EXAMPLES therefore particularly point out embodiments of the presently disclosed subject matter and are not to be construed as limiting in any way the remainder of the disclosure.

### Materials and Methods for the Examples

Cell Lines: The T cell lymphoma cell lines HH and H9 (both cutaneous T cell lymphomas; CTCL), SUP-T1 (T cell Lymphoblastic Lymphoma) and SUP-M2 (ALK-negative anaplastic large cell lymphoma; ALCL), were obtained from ATCC. FEPD is a gift from Dr. Salvia Jain. NKL (natural killer cell lymphoblastic leukemia/lymphoma) cell line was kindly provided by Dr. Howard Young at the National Cancer Institute. The cutaneous melanoma cell line FM3-29 was obtained from DSMZ. The Large Granular Lymphocyte (LGL) leukemia cell line TL-1 was generated in the lab of Dr. Thomas Loughran (Ren et al., 2013). All cells were grown at 37° C. and 5% CO2 in a humidified incubator. Cell lines were authenticated by short tandem repeat DNA profiling (Genetica DNA laboratories) and tested for mycoplasma contamination routinely using the MYCOALERT® PLUS detection kit (Lonza Catalog No. LT07-710) and Mycoplasma detection kit from Southern Biotech. Experiments were performed within 6 weeks of thawing. HH, H9, SUP-T1, FEPD and FM3-29 cells were cultured in RPMI-1640 (Corning, Glendale, AZ) with 10%-20% FBS (Thermofisher Scientific, Waltham, MA); SUP-M2 cells were cultured in RPMI-1640 with 20% FBS; TL-1 cells were cultured in RPMI-1640 with 10% FBS and supplemented with 200 U/mL IL-2 (Miltenyi Biotec Catalog No. 130-097-743) and NKL cells were cultured in RPMI-1640 with 10% FBS and supplemented with 100 U/mL IL-2.

Chemicals: Romidepsin was purchased from eNovations Chemicals LLC, mPEG-PDLLA and mPEG-PLGA were purchased from AKiNA PolySciTech, 3,3′-dioctadecyloxacarbocyanine perchlorate (DiO) was procured from Cayman chemical company and Poloxamer 188 was acquired from Sigma Aldrich. Acetonitrile was purchased from Fisher Scientific. Amicon ultra centrifugal filters of MWCO of 30 kDa, 50 kDa and 100 kDa were purchased from Millipore, Sigma.

Fabrication of Nanoromidepsin: We adopted a tandem parallel synthesis approach in order to achieve optimal physicochemical properties (>500 μg/mL romidepsin, <100 nm particle size, and <0.2 polydispersity index (PDI)) using a versatile nanoprecipitation method (see PCT International Patent Application Publication No. WO 2023/06463, which is incorporated herein by reference in its entirety). We explored the influence of selected parameters of the nanoprecipitation method including (but not limited to) solvent to anti-solvent ratio and drug to polymer ratio to produce romidepsin loaded nanoparticles meeting the determined criteria. Briefly, Nanoromidepsin was synthesized using romidepsin, diblock copolymers either mPEG-PDLLA or mPEG-PLGA, and the surfactant poloxamer 188. Constituents were dissolved in acetonitrile and added dropwise at a defined rate by a syringe pump into water while stirring. Solutions were left to evaporate for 3-4 hours to allow nanoparticle assembly. The nanoparticle colloidal solution was processed to remove unencapsulated drug and polymer by using 100 kDa molecular weight cut-off centrifugal filters at 1460 relative centrifugal force (rcf) at 25° C. for 20-60 minutes. The purified samples were collected from the filter and reconstituted into ultrapure Milli-Q water or PBS (Nanoromidepsin PDLLA H2O and Nanoromidepsin PDLLA PBS respectively), and analyzed using dynamic light scattering (DLS) and mass spectroscopy. For biodistribution studies, DiO, a fluorescent hydrophobic dye, was dissolved in DMSO and used as a stock solution and diluted with acetonitrile to a concentration of 1 mg/mL, then mixed with the drug and polymer solution. The drug to dye ratio was maintained at a ratio of 10:1 w/w. Co-loaded Nanoromidepsin-DiO polymer nanoparticle and ghost-DiO polymer nanoparticles were prepared using a similar method as described above.

Encapsulation Efficiency: The encapsulation efficiency (EE) of romidespsin in Nanoromidepsin was determined after the PNPs were dissolved in methanol. The concentration of entrapped romidepsin was estimated using LC-MS/MS. The following equation was used to calculate the encapsulation efficiency: EE (%)=(Weight of the drug loaded in the polymeric nanoparticles)/ (Weight of the drug initially used in the fabrication of the nanoparticle)×100

Dynamic Light Scattering: Hydrodynamic size (diameter) and Polydispersity Index (PDI) of the PNP was measured in aqueous solutions using dynamic light scattering (DLS; Malvern Instruments model ZEN 3690, Malvern, Worcestershire, WR141XZ, United Kingdom) at 25° C. This measurement includes the intensity-weighted average diameter of the particles (Z-avg), PDI, the volume-weighted average diameter over the major volume peak (Vol-Peak) and its percentage of the total population (Vol-Peak % Vol).

Electron Microscopy: The size and morphology of Nanoromidepsin and ghost PNP were measured using a FEI Tecnai F20 (FEI, Hillsboro, OR) transmission electron microscope operating as a 120 kV cryo-Electron microscopy (cryo-EM). The cryo-EM samples were prepared using a standard vitrification method. An aliquot of ˜3 μl sample solution was applied onto a glow-discharged perforated carbon-coated grid (2/1-3C C-Flat; Protochips, Raleigh, NC, USA) where the excess solution was blotted with filter paper. The samples were then quickly plunged into a reservoir of liquid ethane at −180 C. The vitrified samples were stored in liquid nitrogen and transferred to a Gatan 626 cryogenic sample holder (Gatan, Pleasentville, CA) and then maintained in the microscope at −180 C. All images were recorded with a Gatan 4K×4K pixel CCD camera under cryo-condition at a magnification of 9,600× or 29,000× with a pixel size of 1.12 nm or 0.37 nm, respectively, at the specimen level, and at a nominal defocus ranging from −1 to −3 μm. The unfiltered samples were recorded at 9,600×.

Liquid Chromatography and Mass spectrometry: Romidepsin quantification: Internal standard (romidepsin-d7), was spiked into 25 μL of plasma and the protein precipitated by the addition of 100 μl of cold 5:3 methanol: acetonitrile and centrifuged for 10 minutes at 4° C. and 13000× g. The supernatant was subsequently analyzed by liquid chromatography-mass spectrometry. Liquid chromatography was performed on an I-class Acquity (Waters, Milford, MA) with an Acquity BEH C18 2.1×50 mm 1.7 μm column maintained at 50° C. The mobile phases were water or methanol, each with 0.1% formic acid. The flow (0.5 mL/min) was maintained at 5% methanol for 5 minutes before a linear gradient to 85% over 4 minutes. The column was subsequently washed in 100% methanol for 2 minutes before being equilibrated at starting conditions. The elute was analyzed by a Waters TQS mass spectrometer with the capillary set at 2.00 kV, desolvation temperature: 500° C., and desolvation gas flow: 900 L/hr, multiple reaction monitoring was used to analyze romidepsin (541.4>424.2 (quantifier) and 272,4, (qualifier)) and romidepsin-d7 (548.2>424.4) with argon as the collision gas. Peak areas were determined in TargetLynx against a standard curve prepared in plasma. Romidespsin concentration in all nanopolymers was quantified similarly with a shorter chromatography gradient from 20% methanol to 100% methanol over 1 minute, held for 0.5 minutes, before re-equilibrating for an additional 0.5 minutes.

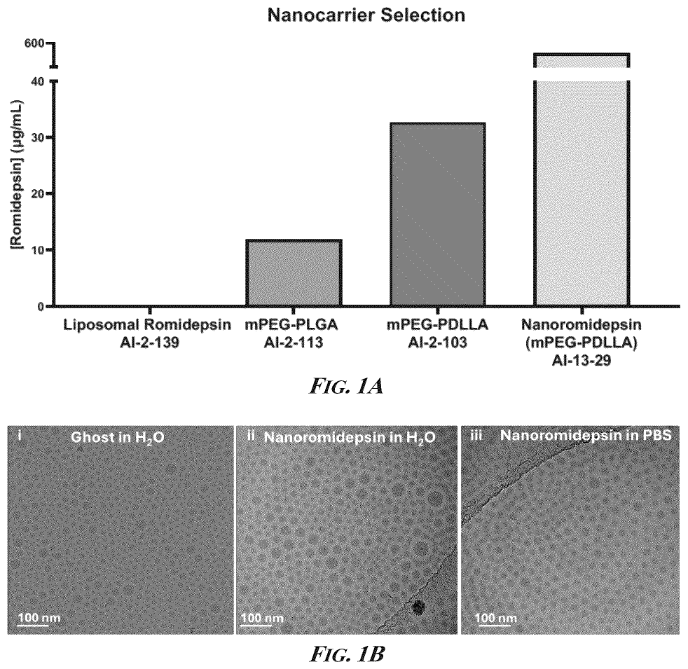

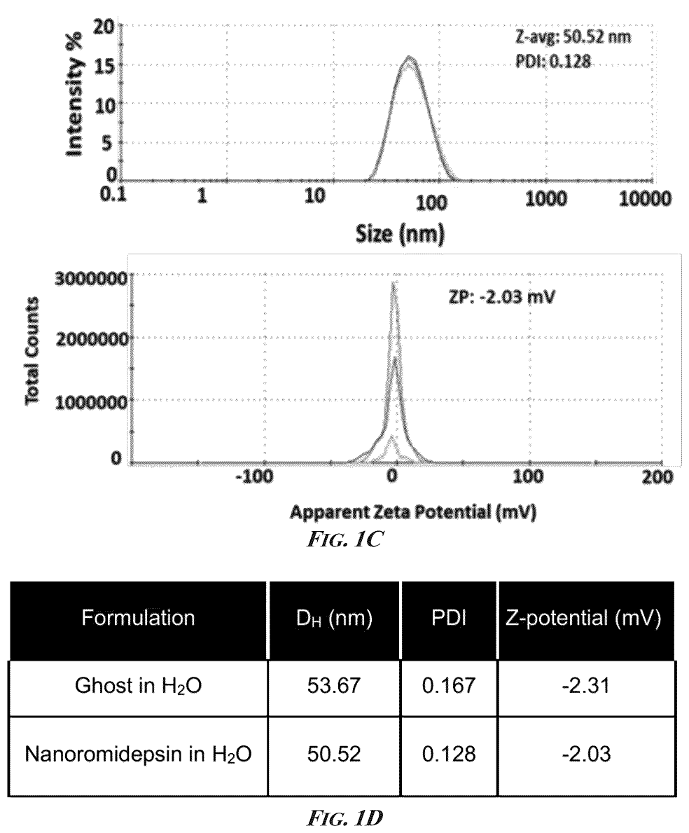

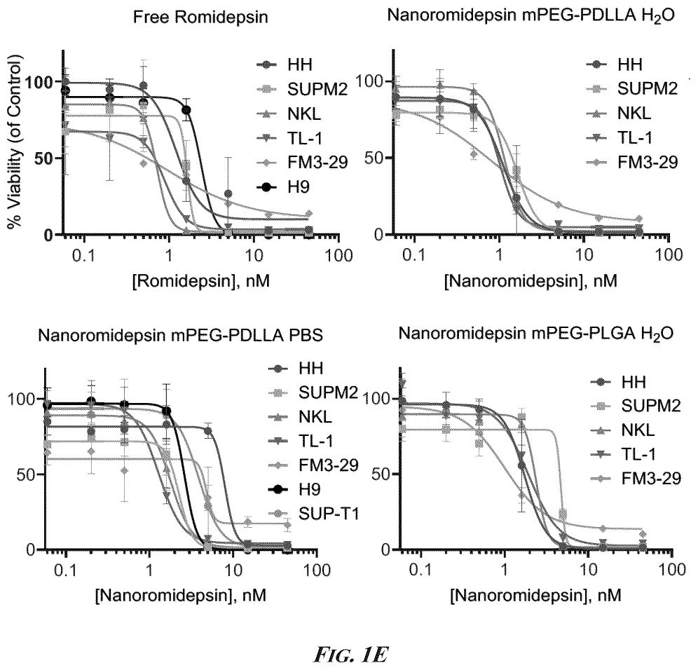

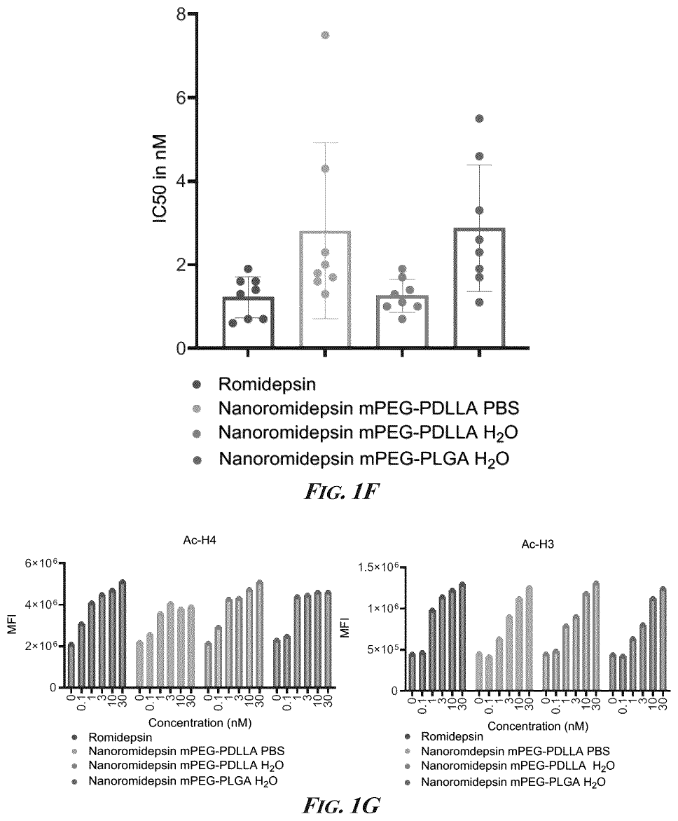

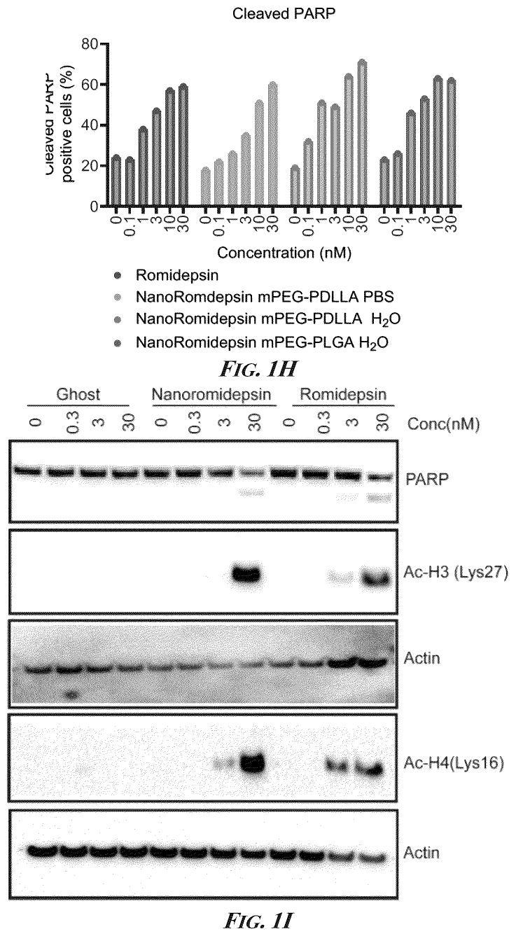

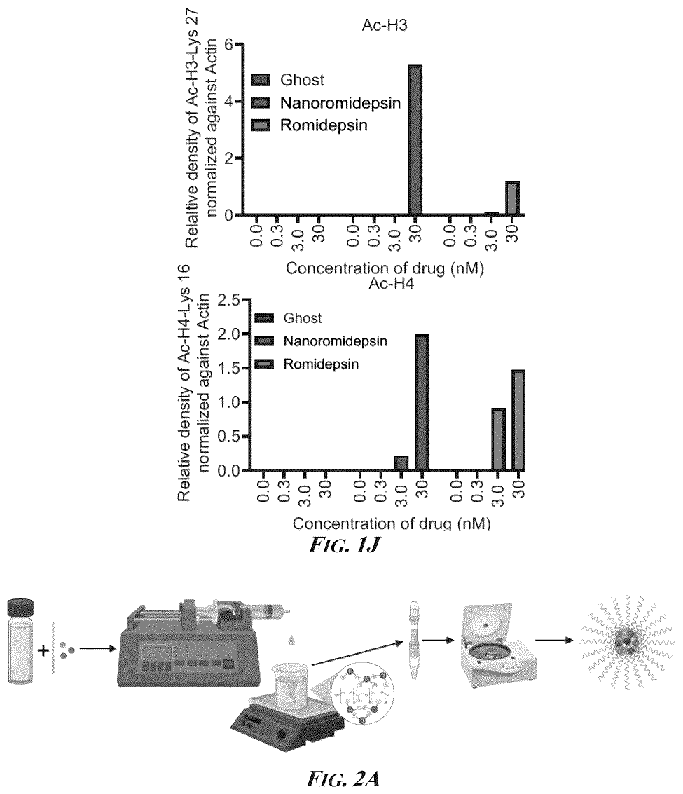

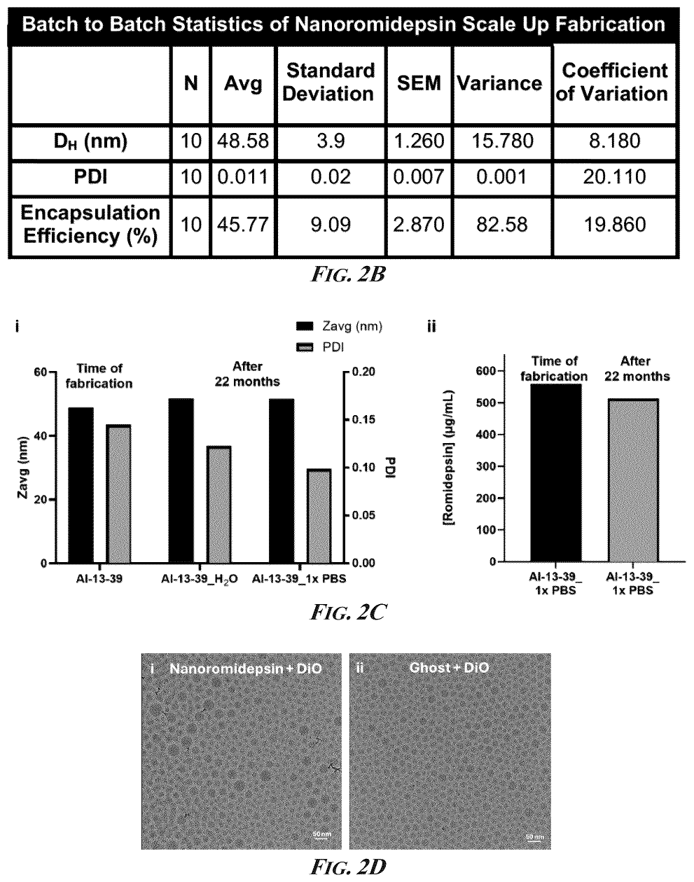

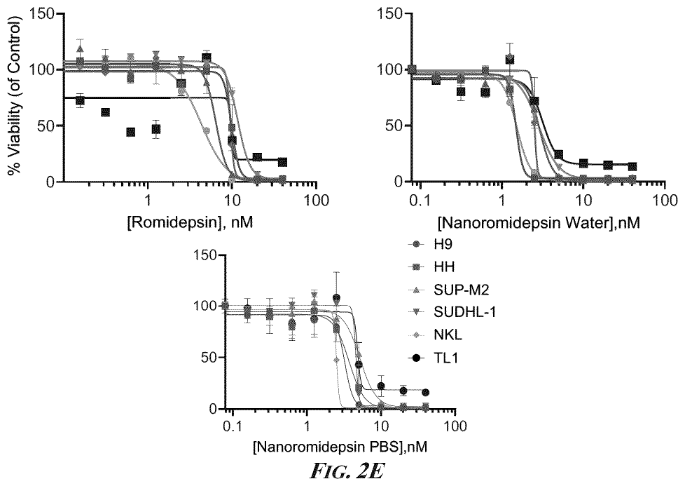

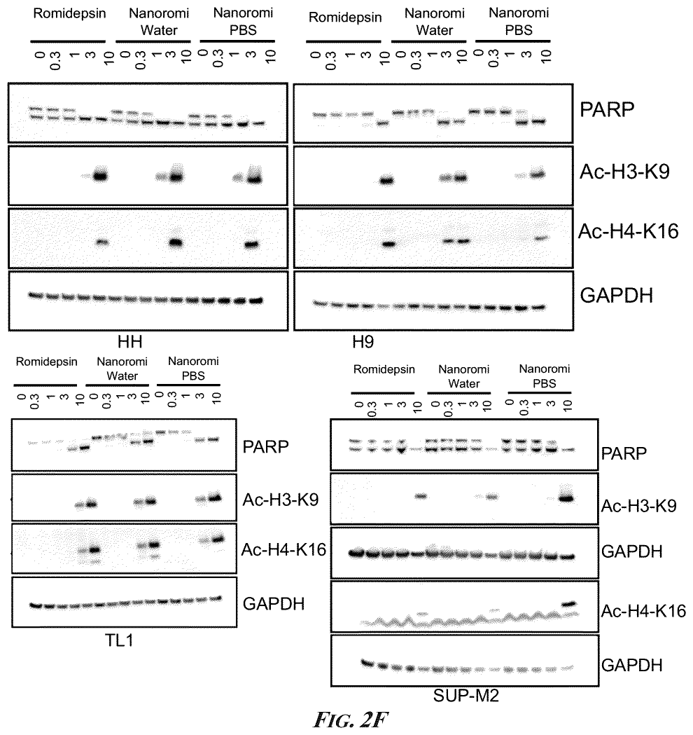

Cell Viability Assay: Cell lines were plated at the appropriate cell densities (SUP-T1, HH, H9, FEPD, NKL and TL-1 at 100,000 cells/ml/well and SUP-M2 and FM3-29 at 50,000 cells/ml/well) in a 48-well plate. Nanoromidepsin mPEG PDLLA PBS, Nanoromidepsin mPEG PDLLA H2O, Nanoromidepsin mPEG PLGA H₂O, or free romidepsin were added to the cells at concentrations ranging from 0.03 nM to 30 nM with a two to three-fold serial dilution. Ghost PNP of mPEG PDLLA and mPEG PLGA were added to the various cell lines as a standard control at varying dilutions in order to achieve the same concentrations of the polymers as replicated in the romidepsin PNP. Cells were harvested following 60 (FIG. 1), and 48 hours (FIG. 2) of drug exposure at 37° C., 5% CO₂ and assayed for cell viability using CELLTITER-GLO®® (Promega) by following manufacturer instructions. Briefly, approximately 15 minutes after the addition of CELLTITER-GLO®® (at a 1:1 ratio), luminescence was read on a GLOMAX® Discover Microplate Reader (Promega). Luminescence was normalized to untreated control which was defined as 100% viability.

Flow Cytometry: Cells were harvested after 30 hours of treatment, washed twice with PBS, fixed with 4% paraformaldehyde solution and permeabilized with 70% methanol before incubating with the relevant antibodies, including Rabbit anti-HDAC1 polyclonal antibody (Proteintech; Cat no 10197-1-AP), Rabbit anti-Acetyl Histone H3 K9/K14 polyclonal antibody (Millipore Sigma; Catalog No. 06-599) and Rabbit Anti-Acetyl Histone H4 polyclonal antibody (Millipore Sigma; Catalog No. 06-598). After 30 minutes of incubation at room temperature cells were washed twice with PBS and FITC conjugated secondary Goat anti-rabbit polycclonal antibodies (Thermofisher; Catalog No. 31635) were added. Cells were incubated on ice for 30 minutes and then washed in PBS. After the second wash, cells were resuspended in PBS with 4% paraformaldehyde prior to signal acquisition on a flow cytometer (Attune, Thermo Fisher Scientific). Apoptosis in cell lines was assessed by the presence of cleaved Poly (ADP-ribose) polymerase-1 (PARP) using a PE conjugated mouse anti-cleaved PARP (Asp214) monoclonal antibody (BD Biosciences: Catalog No. 552933, clone F21-852). After staining, the cells were acquired using an Attune Flow Cytometer and the data analyzed using FloJo analysis software. The Mean Fluorescence intensity (MFI) was indicative of the level of expression of each marker.

Western Blots: Cells were incubated with the indicated concentrations of each drug and PNPs under normal growth conditions for 24 hours. Proteins from total cell lysates were resolved on 12% to 20% SDS-PAGE and transferred to nitrocellulose membranes. Membranes were blocked in phospho-buffered saline containing 0.05% Triton X-100 containing 5% skim milk powder, which were then probed overnight with specific primary antibodies. Antibodies were detected with the corresponding horseradish peroxidase-linked secondary antibodies. Blots were developed using Bio-Rad CLARITY™ and CLARITY MAX™ ECL chemiluminescent substrate detection reagents. Signal detection and imaging was captured and analyzed using a CHEMIDOC™ System (Bio-Rad) with signal quantification. The monoclonal and polyclonal antibodies used were as follows: acetylated histone H3, acetylated histone H4, vinculin, and β-actin (all from Cell Signaling Technology).

Generation of H9-dTomato-luc Cell Line: pUltra-Chili-Luc was purchased from Addgene (Catalog No. 48688; Fujiwara et al., 2021) and was transfected into HEK293T/17 cells with lentiviral packaging plasmids VSV-G, PLP1, and PLP2 (Invitrogen) by Lipofectamine 3000 (Invitrogen; a gift from Dr. Su-Fern Tan, University of Virginia). Viral particles were collected after 48 and 72 hours post transfection. Virus particles were added to cells every 24 hours for 2 days with 7 μg/ml polybrene. Transduced cells were sorted by flow-cytometer with strongest dTomato expression and maintained in 20% FBS.

Single and Mutiple Dose In Vivo Toxicity Study: 5 to 7 week old female BALB/c mice (n=5) were randomly assigned to treatment cohorts based on body weight. Mice were divided into two cohorts: (i) Nanoromidepsin or (ii) free romidepsin. Mice from both cohorts were administered a single treatment of 1, 2, 3, 5, or 8 mg/kg body weight by intraperitoneal (IP) injection, and 1, 2, 3, 5, 8 or 10 mg/kg body weight by intravenous (IV) administration. The mice were monitored for 14 days post-treatment. For repeat dose maximum tolerated dose (MTD) studies, three million H9 cells expressing a fluorescent protein, Chili (dTomato-absorption max and emission max at 554 nm and 581 nm, respectively) and the bioluminescence generating protein firefly luciferase (Luc), were injected subcutaneously into the right flank of 5 to 7 week old female NSG mice (NOD-Cg-Prkdcscid Il2rgtm1 Wjl/SzJ mice; The Jackson Laboratory). After the tumor reached the bioluminescent intensity (photon/s/cm²/sr) of 10⁶ or higher, the H9-dTomato-luc xenograft-containing NSG mice were treated IV as follows: (i) 2 or 3 mg/kg ghost PNP (equivalent dose), free romidepsin or Nanoromidepsin twice a week for 2 weeks; (ii) 2.5 mg/kg ghost PNP (equivalent dose), free romidepsin or Nanoromidepsin once a week for three weeks; (iii) 4 mg/kg ghost PNP (equivalent dose), free romidepsin or Nanoromidepsin once a week for three weeks, and (iv) 5 or 8 mg/kg ghost (equivalent dose), free romidepsin or Nanoromidepsin once every two weeks. The mice were sacrificed the day after the last treatment. Nanoromidepsin was diluted under sterile conditions in phosphate buffered saline (PBS). Romidepsin was prepared in DMSO and then diluted with PBS under sterile condition. For single dose and repeat dose toxicity studies, weight loss and clinical score were quantitated as a function of time. Clinical signs were scored by observing activity, appearance (hair coat and eyes/nose), posture, and body condition with a maximum of 3 points ascribed to each criterion (0, normal; 1, slight deviation from normal; 2, moderate deviation from normal, 3, severe deviation from normal). Criteria leading to euthanasia included weight loss of >20% or a clinical score >6.

Pharmacokinetic Study: 5 to 7 week old female BALB/c mice were divided into two treatment cohorts including Nanoromidepsin or free romidepsin. Each treatment cohort was further divided into two sub-cohorts depending upon the route of administration (IP or IV). Each sub-cohort (n=21) received a single treatment of one half MTD as defined from our single dose toxicity study (2.5 mg/kg body weight) of Nanoromidepsin or free romidepsin. Mice were sacrificed (n=3 per time point) at 1, 3, 6, 18, 24, 48, and 72 hours after the treatment. Blood (˜1 mL) was collected via terminal cardiac puncture under isoflurane anesthesia and collected in EDTA-coated K3EDTA tubes followed by centrifugation (2,000× g for 15 minutes) to isolate plasma. Plasma was placed in cryopreservation vials and preserved by snap freezing using liquid nitrogen. Blood from three untreated mice was collected at the beginning of the treatment (T=0) and used as a control. The level of romidepsin was quantified by a validated method based on reversed-phase liquid chromatography coupled to tandem mass-spectrometric detection with a standard curve derived with stock romidepsin, as described above. Blood was collected by sub-mandibular bleeding after 1 and 24 hours following the last treatment with 4 mg/kg free romidepsin and Nanoromidepsin as described above in the repeated dose study. Plasma was collected and the romidepsin was quantified as described above. Liver and tumor were harvested, fixed in formalin for pathological analysis following H&E staining and processed for LC-MS based quantification of romidepsin.

Biodistribution Study: To measure the biodistribution of Nanoromidepsin, NSG mice were injected with H9-dTomato-luc cells subcutaneously. The tumor growth was monitored by in vivo bioluminescence intensity up to twice weekly. Prior to imaging, each mouse was injected by the IP route with the bioluminescence substrate (Nanolight luciferin, 135 mg/kg body weight was given by IP with an injection volume of approximately ˜200 ul). After the tumor reached a bioluminescence intensity (photon/s/cm²/sr) of 10⁶ or higher, tumor-bearing NSG mice were randomly assigned into two groups (n=3) and injected intravenously with Nanoromidepsin co-loaded with DiO (fluorescent dye) or free DiO at an equivalent dose (3.7 mg/kg). Whole-body fluorescence imaging was performed at predetermined times on a cryogenically cooled Lago X (Spectral Instruments Imaging system). Three mice from each group were sacrificed after 72 hours. Tumor and all vital organs were harvested. An ex vivo imaging was performed of tumor and other organs.

Survival and Efficacy Study: H9-dTomato-luc cells were injected subcutaneously into the right flank of 5 to 7-week-old female NOD Cg-Prkdcscid Il2rgtm1Wjl/SzJ mice (The Jackson Laboratory). Mice were imaged twice a week starting six days after inoculation of cells. Once tumors reached the pre-specified bioluminescence intensity (>10⁶ photon/s/cm²/sr), mice were randomized to four treatment groups of 9 mice each: (i) control group treated with normal PBS; (ii) ghost PNP; (iii) romidepsin (3.5 mg/kg), or (iv) Nanoromidepsin (3.5 mg/kg). All drugs were administered by tail vein once a week. Baseline imaging data were recorded for all mice the day before the first dose of the drug. The number of animals, study design, and treatment of animals were reviewed and approved by the Institutional Animal Care and Use Committee (IACUC) of the University of Virginia, Charlottesville. In vivo BLI analysis was conducted on a cryogenically cooled Lago X (Spectral Instruments Imaging system). A second efficacy/survival study was performed using similar methods with 4 different treatment groups of 9 animals each, including: (i) control group treated with normal PBS; (ii) ghost PNP; (iii) romidepsin (4 mg/kg) and (iv) Nanoromidepsin (4 mg/kg).

Ex vivo Cytotoxicity and Flow Cytometry Study: Primary LGL leukemia cells were collected from patients (LGL Leukemia Registry at University of Virginia) who met the criteria of T-LGL (CD3+/CD8+) leukemia with increased numbers of LGL cells in the peripheral blood. Peripheral blood samples were obtained and informed consent signed for all LGL leukemia patients according to protocols approved by the Institutional Review Board of University of Virginia School of Medicine. PBMCs were isolated by Ficoll-Paque gradient separation and viably cryopreserved (LeBlanc et al., 2020). Samples with viability >90% were then used for further downstream analysis. PBMC were seeded with 100,000 cells/well in a 96 well plate and treated with indicated doses of ghost PNP, romidepsin and NanoRomidepsin. Cytotoxicity assay was performed using Cell Titer Glo as described above. Flow cytometry analysis was performed as described earlier (Jayappa et al., 2017; Jayappa et al., 2021). Briefly, PBMCs from the same patients used in the cytotoxicity assay were treated with the indicated doses of ghost PNP, romidepsin and Nanoromidepsin for 48 hours. The PBMCs were stained with Live/Dead near-infrared viability dye. Cells were then fixed in paraformaldehyde (1.6%) and stained for surface markers using anti-CD3BV605(clone OKT3), anti-CD8 BV421(clone SK1), and anti-CD57 BB515 (clone NK-1), for overnight at 4° C. PBMCs were washed and permeabilized using saponin and stained with anti-Cleaved PARP-PE clone (F21-852). Flow cytometry data analysis was performed using FlowJo software.

Statistical Analysis: Results are presented as the mean±SD, unless indicated otherwise. Statistical significance was determined by 1-way ANOVA or 2-tailed Student's t test or log rank test, unless specified otherwise, using GraphPad Prism software, and a p-value of less than 0.05 was considered statistically significant.

### Example 1

### Engineering of Nanoromidepsin and DiO Loaded Polymer Nanoparticles (PNP)

Several different NPs of romidepsin were synthesized using generally regarded as safe (GRAS) amphiphilic di-block copolymers and FDA approved lipids for liposomes. Liposomes did not achieve romidepsin encapsulation and were not pursued further. PNPs were synthesized using mPEG-PDLLA and mPEG-PLGA, and the surfactant poloxamer-188 using a solvent displacement or nanoprecipitation technique as described above. LC/MS confirmed an average romidepsin concentration in liposomes and polymer nanoparticles of <0.1μg/mL and >500 μg/mL respectively. (FIG. 1A). m-PEG-PDLLA nanoparticles exhibited higher drug concentrations of approximately 540μg/mL with an average EE of 48%. Cryo-EM (FIG. 1B) revealed that both ghost and romidepsin loaded PNPs exhibited uniform spherical morphology, homogeneous size of 35 nm with minimal to no agglomeration. DLS (FIGS. 1C and 1D) revealed a unimodal distribution of the particles with an average size of 46.25 nm and a PDI of 0.145.

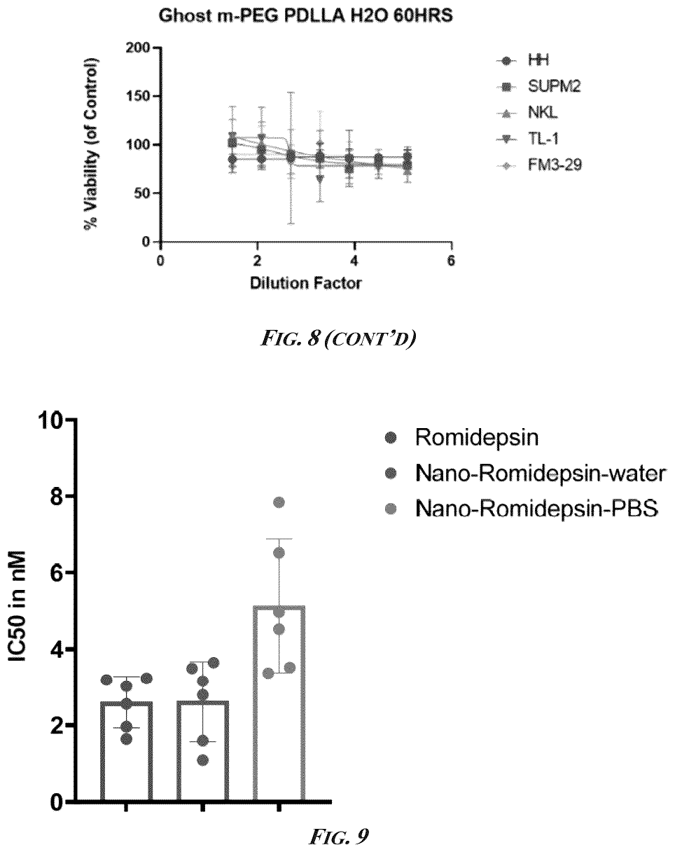

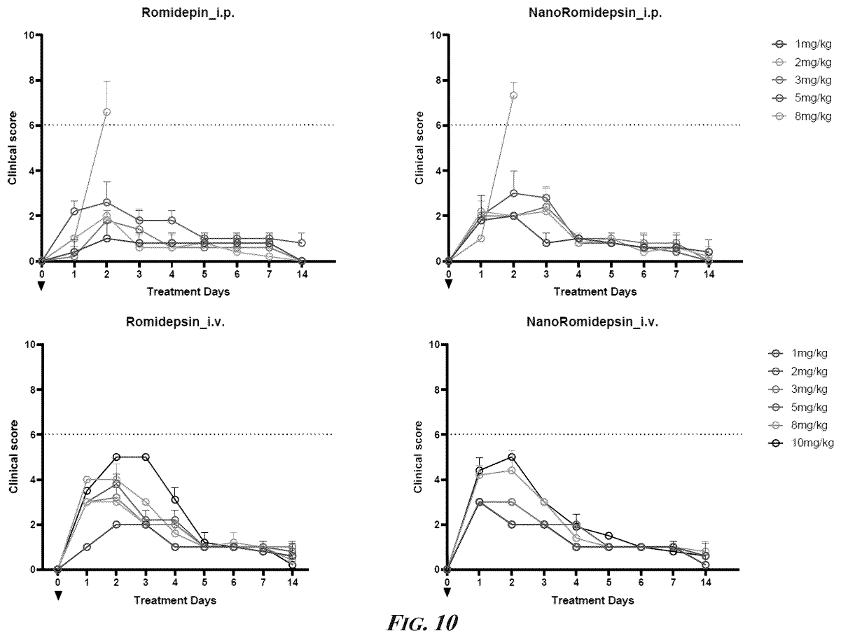

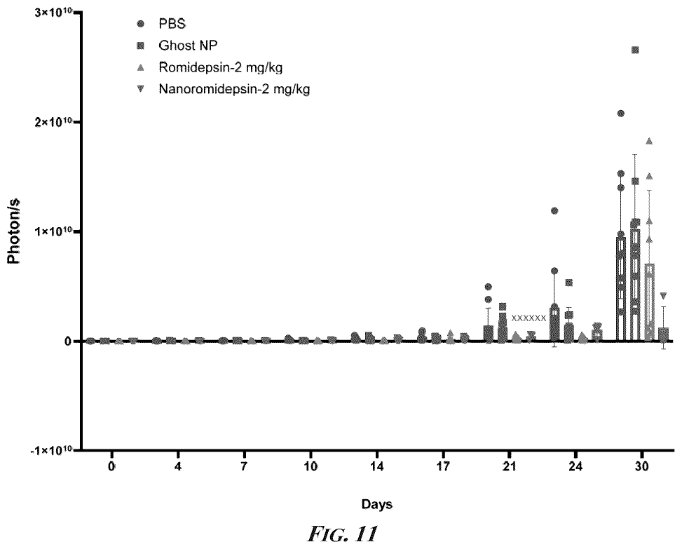

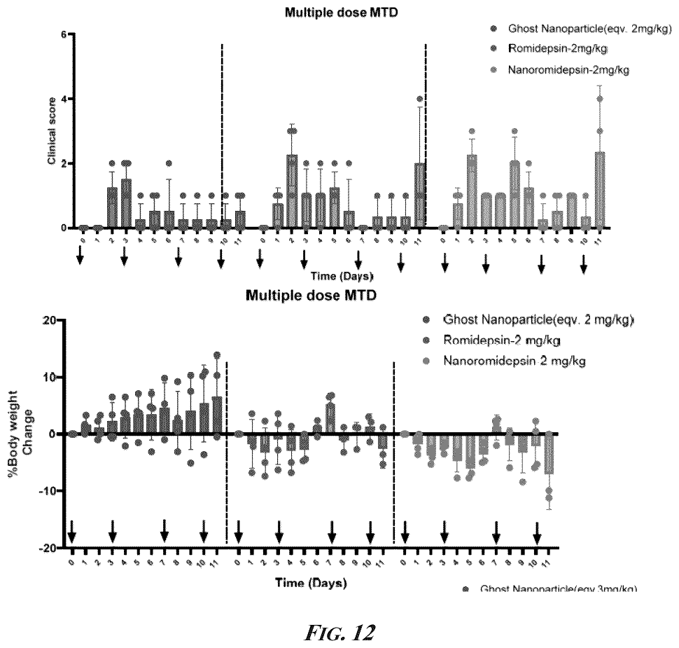

The concentration-response relationship for each of the PNP was determined and compared to free romidepsin across a panel of T-cell lymphoma and LGL leukemia cell lines, as well as a melanoma cell line to understand activity in a solid tumor malignancy (FIG. 1E). The IC₅₀ for each PNP is shown in FIG. 1F. Ghost PNPs were diluted to match the volume of the polymer shell in the Nanoromidepsin derivatives (FIG. 8), and consistently revealed minimal to no toxicity in the in vitro assays. All three PNPs of romidepsin inhibited cell growth of all cancer cell lines in a concentration dependent manner (FIG. 1E), though the IC₅₀ values for the three different PNPs varied across the cell lines tested. At 60 hours, most cell lines were consistently sensitive to Nanoromidepsin m-PEG PDLLA H₂O (IC₅₀=0.7-1.9 nM) which was very similar to romidepsin (IC₅₀=0.6-1.9 nM; FIG. 1F). Both Nanoromidepsin m-PEG PDLLA PBS (IC₅₀=1.3-7.5 nM) and Nanoromidepsin m-PEG PLGA H₂O (IC₅₀=1.1-5.5) were slightly less potent compared to romidepsin and Nanoromidepsin m-PEG PDLLA H₂O. There was no inhibition of growth in any cell line with the corresponding ghost PNP. (FIG. 8). Flow cytometry and western blotting demonstrated that treatment with all three romidepsin PNPs induced apoptosis similar to romidepsin as shown by an increase in the expression of cleaved PARP (FIGS. 1H and 1I). However, Nanoromidepsin m-PEG-PDLLA PBS and Nanoromidepsin m-PEG PDLLA H₂O demonstrated higher PARP cleavage compared to romidepsin and Nanoromidepsin m-PEG-PLGA H₂O.

Levels of H3 and H4 acetylation were measured following treatment with increasing concentrations of romidepsin and the three PNPs of romidepsin. A concentration dependent increase in acetylation of both H3 and H4 was observed by flow cytometry when cancer cells were treated with either romidepsin or one of the three romidepsin PNPs (FIG. 1G). Although a similar degree of increased histone acetylation was observed after treatment with all three PNPs, Nanoromidepsin PDLLA H₂O was comparable to free romidepsin in its pattern of histone acetylation. Given that among the three PNPs, Nanoromidepsin PDLLA H₂O exhibited the lowest IC₅₀ and comparable histone acetylation and PARP cleavage compared to free romidepsin, we further validated the flow cytometry data and western blotting analysis with Nanoromidepsin PDLLA H₂O. The levels of acetylation of H3 lysine 27 and H4 lysine 16 were measured following treatment with increasing concentrations of romidepsin and Nanoromidepsin PDLLA H₂O. Western blot analysis demonstrated an increase in H3/H4 acetylation following treatment with 0.3 to 30 nM of romidepsin or Nanoromidepsin PDLLA H₂O at 24 hours (FIG. 1I and IJ). The increased acetylation of both H3 and H4 proteins was higher in cells treated with Nanoromidepsin compared to romidepsin.

Between the mPEG-PLGA and mPEG-PDLLA Nanoromidepsin, the PDLLA Nanoromidepsin showed the best physicochemical properties (size, PDI, and encapsulation) and biological activities, prompting further optimization, scale up, and characterization (data not shown) of the PDLLA Nanoromidepsin.

### Example 2

### Scale-up of Romidepsin PNP Synthesis, Physicochemical Characterization, and In Vitro Activity

The scale up workflow and resulting PNP physicochemical properties are shown in FIG. 2A-B. Several key factors influencing the batch production of romidepsin PNP were identified and optimized for bulk synthesis. These factors include: the polymer type, drug-to-polymer ratio, drug-to-surfactant ratios, diffusion coefficients of solvents, solvent-to-anti-solvent ratio, rate of addition of reagents and processing (evaporation, centrifugation, and reconstitution) of nanoparticles (FIGS. 2A and 2B). These factors play a critical role in the efficiency of drug encapsulation, scalability, nanoparticle characteristics such as size, PDI, drug release, and the nature of discrete biological interactions. The PDLLA H2O Nanoromidepsin revealed particle stability for 22 months at 4° C. (FIG. 2C). Co-loading of romidepsin with DiO produced particles of similar size, indicating this method can potentially accommodate efficient co-packaging of multiple drugs (FIG. 2D).

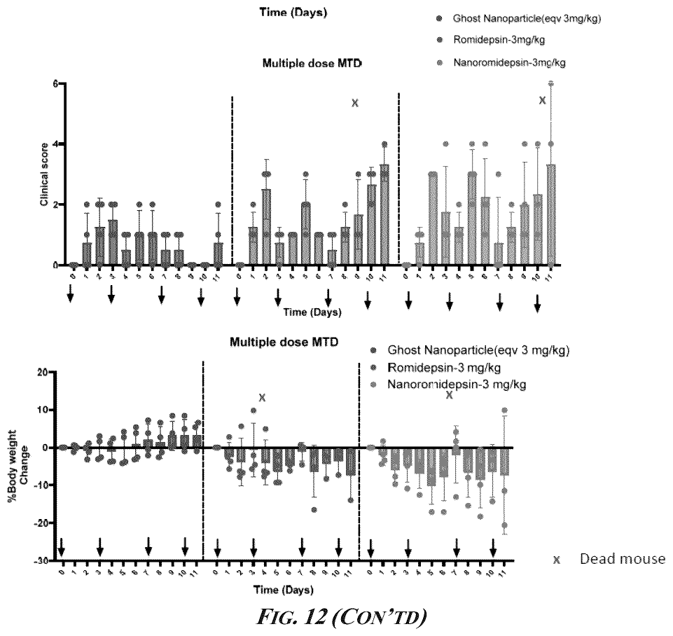

To verify that the scaled-up version of Nanoromidepsin PDLLA H₂O and Nanoromidepsin PDLLA PBS retained similar cell-killing properties without compromising the mechanism of action, we repeated cytotoxicity studies to assess the cytotoxicity of the PNPs across the panel of PTCL and LGL leukemia cell lines (HH, H9, SUP-M2, SUDHL1, TL1, and NKL). All six TCL cell lines showed varying degrees of sensitivity to romidepsin and Nanoromidepsin PNPs after 48 hours of treatment (FIG. 2E). The IC₅₀ of Nanoromidepsin PNPs were ˜2-fold lower (IC₅₀ between 1.5-5 nM) compared to free romidepsin (IC₅₀ value 4-10 nM; FIG. 9.

Next, we performed western blot analysis on four different TCL cell lines to compare the effect of the scaled up Nanoromidepsin PDLLA H2O and PDLLA PBS PNPs on histone acetylation (FIG. 2F). After 48 hours of treatment, Nanoromidepsin PNPs demonstrated superior or comparable H3/H4 acetylation compared to free romidepsin across all cell lines studied. HH, H9, and SUPM2 cell lines demonstrated a substantially greater increase in histone acetylation after treatment with Nanoromidepsin PNPs compared to free romidepsin consistent with the data in FIG. 2E, affirming a relatively lower IC₅₀ value for Nanoromidepsin PNPs FIG. 2F).

### Example 3

### Nanoromidepsin Exhibited Superior Cytotoxicity Against Primary LGL Leukemia Samples

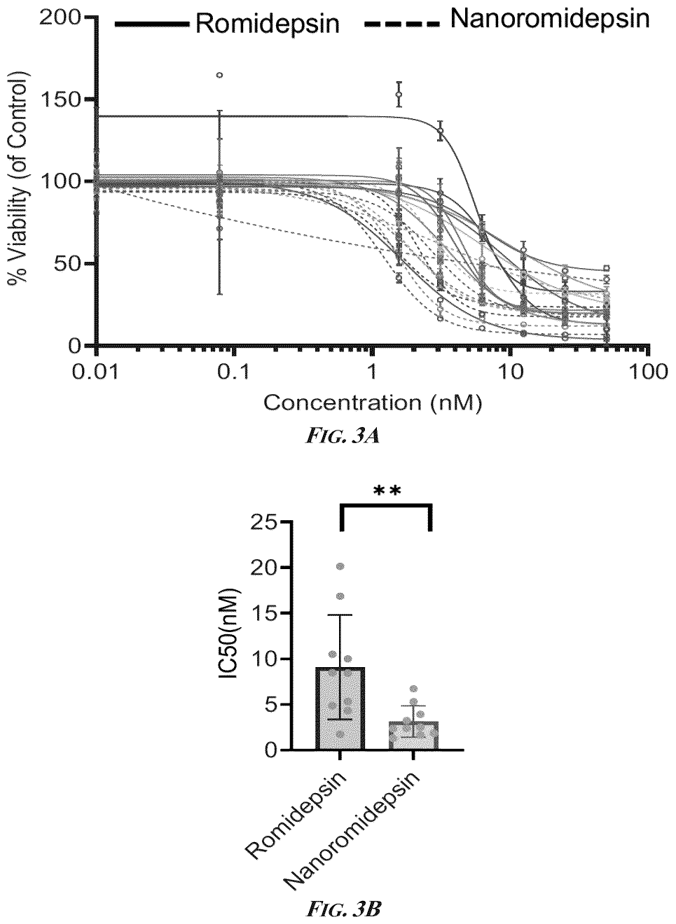

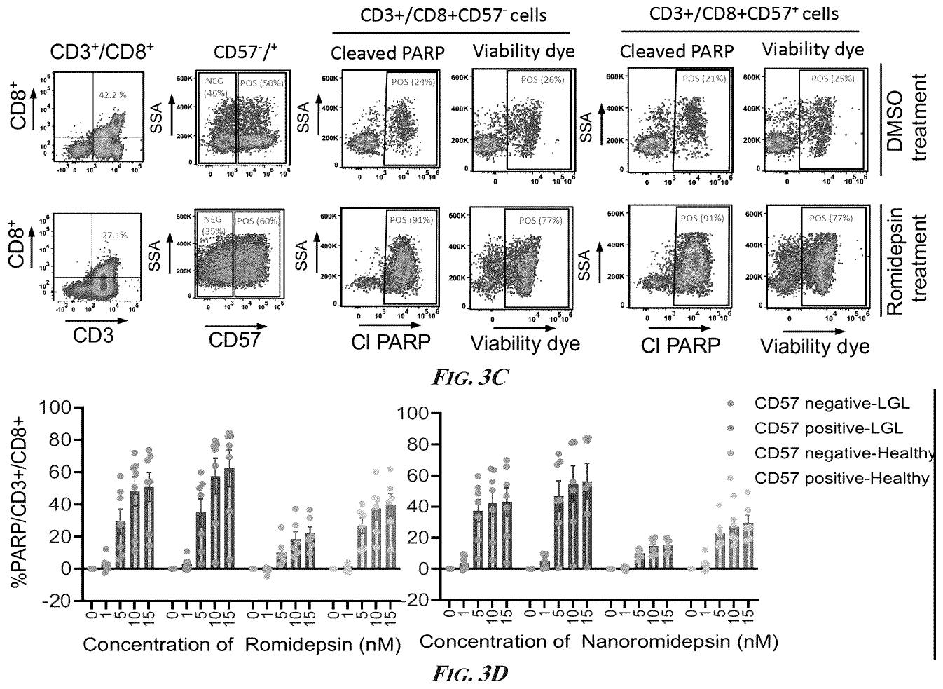

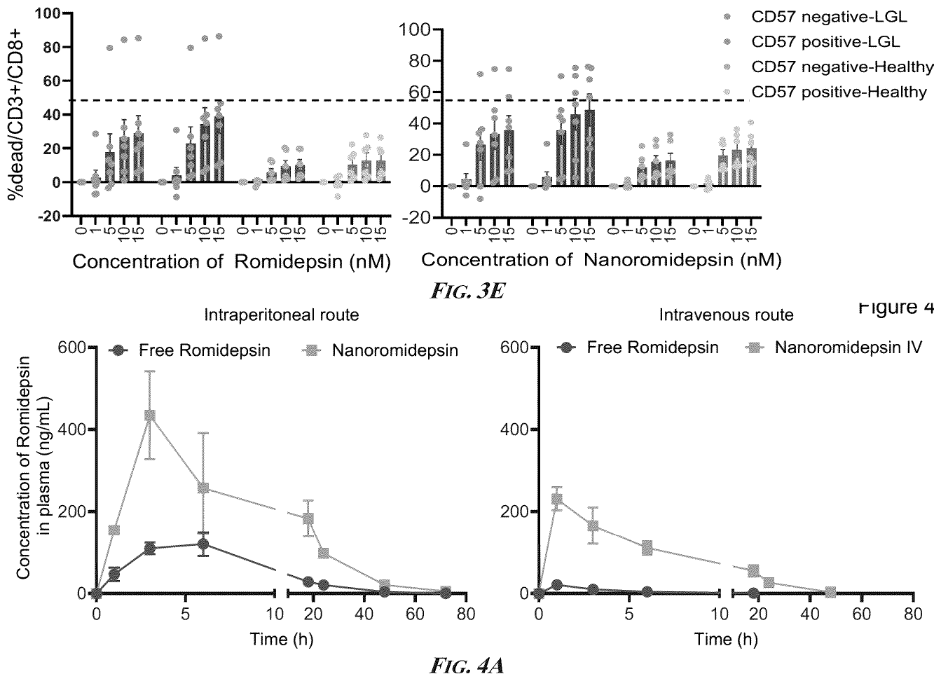

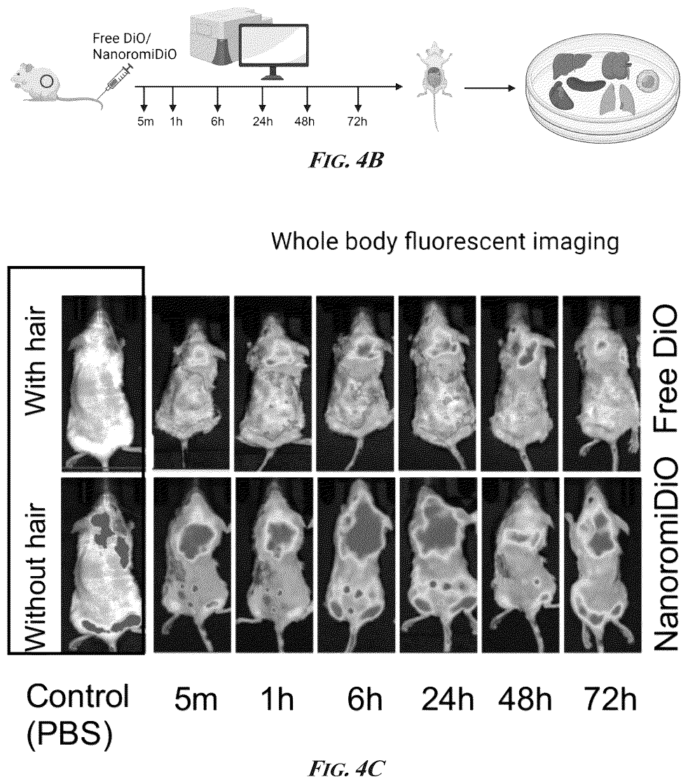

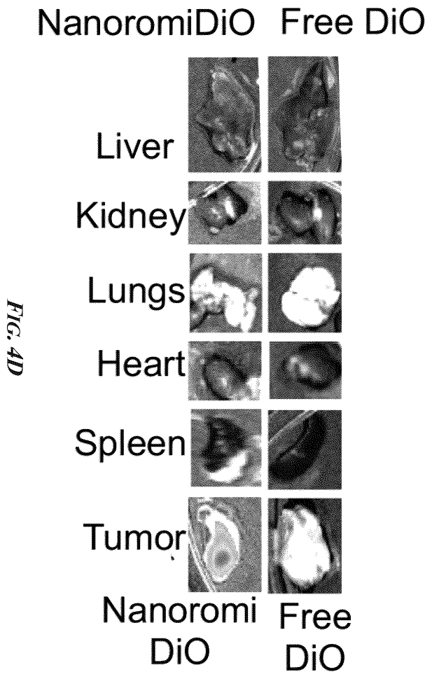

The anti-proliferative effect of free romidepsin and Nanoromidepsin were compared on PBMC isolated from LGL leukemia patients. LGL-leukemia is a rare chronic mature lymphoproliferative disorder that is distributed into two subtypes: T-LGL leukemia and NK-LGL leukemia (Lamy et al., 2017). T-LGL leukemia typically exhibits a CD3+, TCR αβ+, CD4−, CD5dim, CD8+, CD16+, CD27−, CD28+, CD45RO−, CD45RA+, and CD57+ phenotype, which represents a constitutively activated T-cell phenotype (Bigouret et al., 2003; Lundell et al., 2005; O'Malley, 2007). Nanoromidepsin demonstrated superior cytotoxicity in TL1 (a T-cell LGL) and NKL (a NK cell LGL) cell lines, exhibiting a lower IC₅₀ compared to free romidepsin (FIGS. 2A and 2B). An ex vivo cytotoxicity assay performed on PBMC from the LGL-leukemia patients demonstrated that Nanoromidepsin produced a statistically significant improvement in potency, with an IC₅₀ of 3.1±1.7 nM, compared to free romidepsin, which exhibited an IC₅₀ of 9.06±5.7 nM. (p=0.0057; FIGS. 3A and 3B). As whole PBMC samples also contain a small proportion of non-leukemic cells, we designed a multi-color flow cytometry-based functional assay (Jayappa et al., 2021) to quantify apoptosis in CD3+CD8+CD57+ or CD3+CD8+CD57− cell populations of LGL-leukemia patients (FIG. 3C). PBMCs from healthy individuals were used as control. These data revealed that the percentage of CD3+CD8+CD57− and CD3+CD8+CD57+ cells positive for the cleaved PARP apoptosis marker was similar for Nanoromidepsin and free romidepsin treated PBMC samples, though the percentage of dead cells (viability dye+) in CD3+CD8+CD57+ and CD3+CD8+CD57− cells was comparatively higher in the Nanoromidepsin treated PBMC samples although the results were not statistically significant (p<0.5873 and 0.4603 respectively; FIGS. 3C and 3D).

### Example 4

### Nanoromidepsin Demonstrates Superior Pharmacokinetic Parameters and Biodistribution Compared to Free Romidepsin

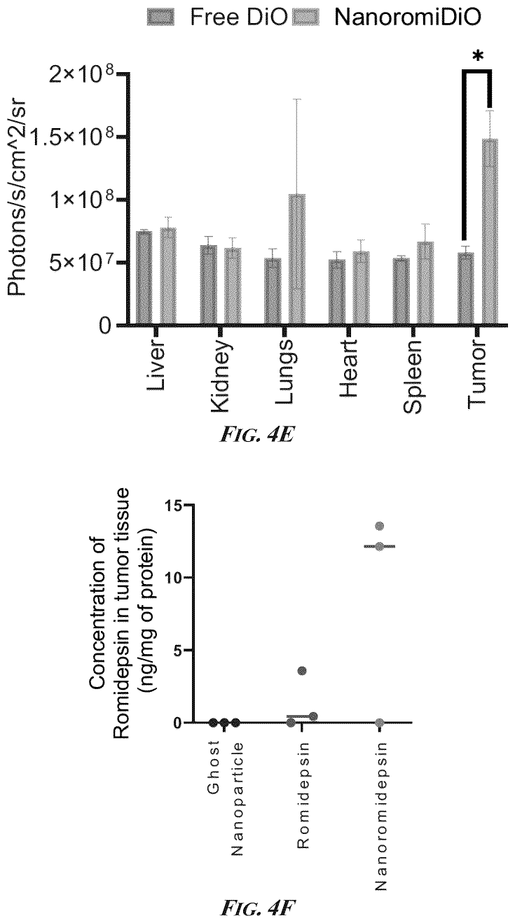

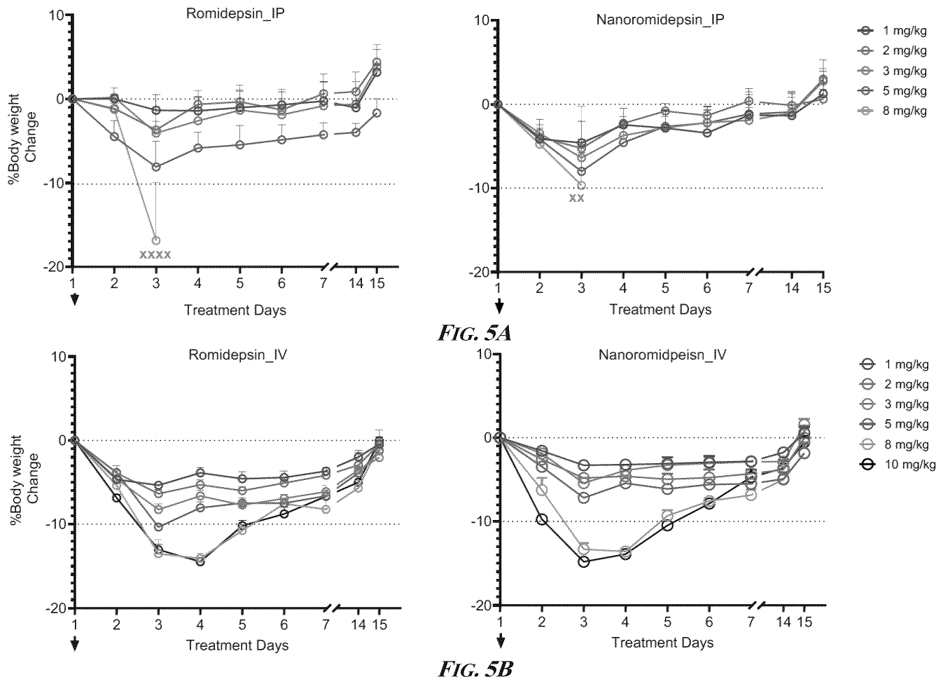

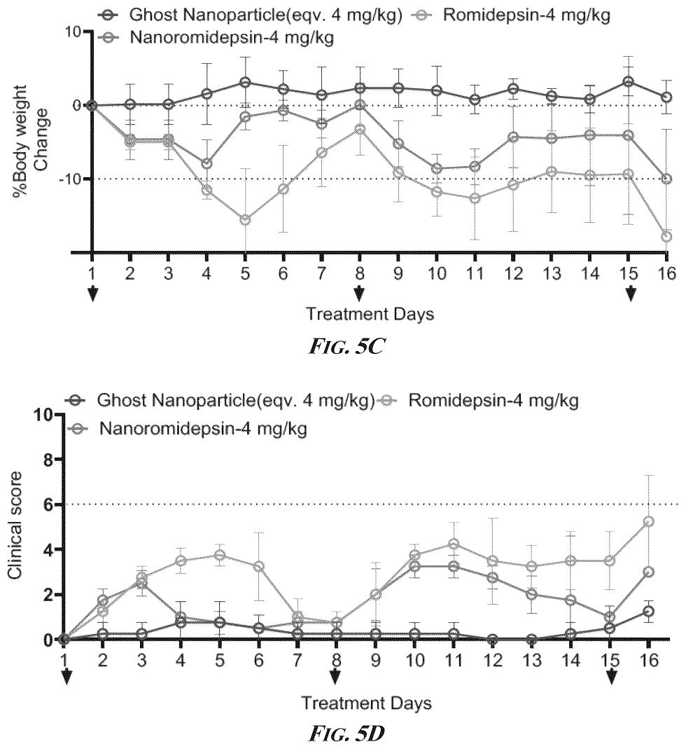

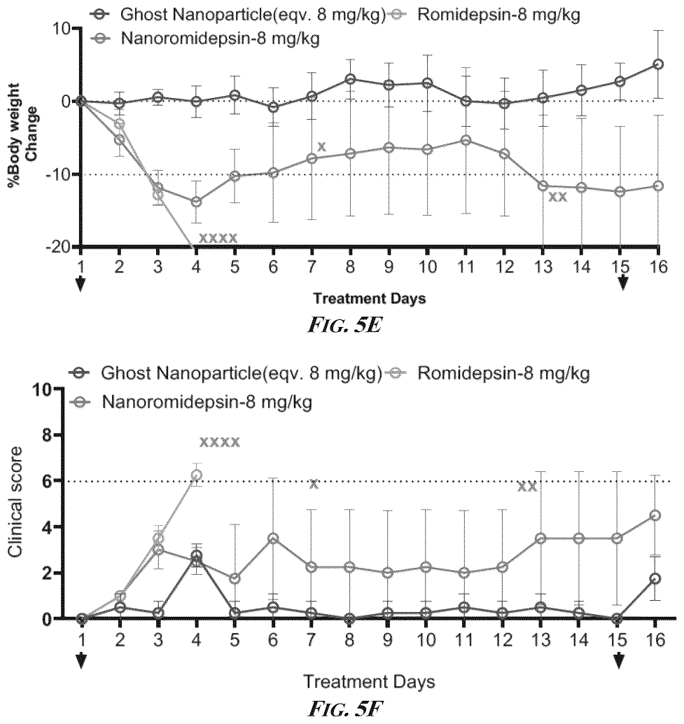

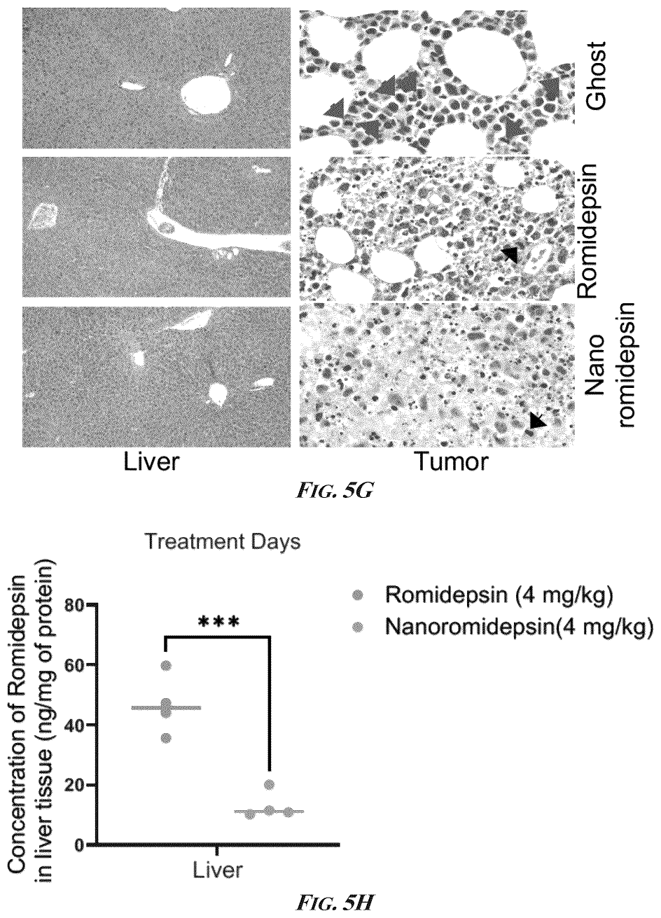

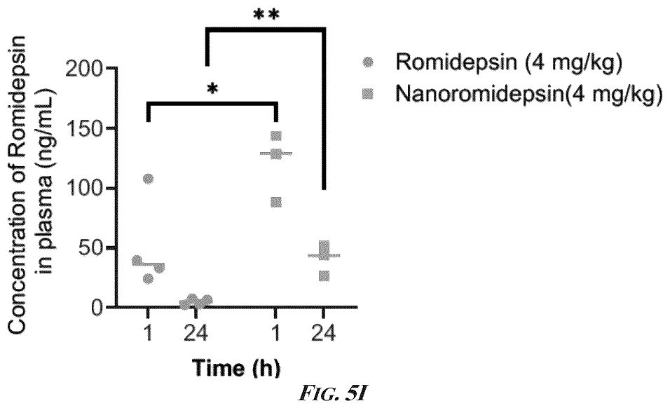

To evaluate the pharmacokinetic profile of the Nanoromidepsin PDLLA PBS (hereafter referred to herein as Nanoromidepsin), BALB/c mice were injected with the indicated drugs by IV or IP administration. The plasma concentration of romidepsin was quantified using LC-MS/MS. Irrespective of the route of administration, the plasma romidepsin concentration of free romidepsin rapidly declined after 6 hours. (FIG. 4A). In contrast, Nanoromidepsin exhibited a higher area under the curve (AUC) of exposure 48 hours post-treatment, irrespective of the route of administration. After IV administration, Nanoromidepsin attained peak drug concentrations (Tₘₐₓ) at 6 hours, while free romidepsin attained a Tₘₐₓ of 3 hours. The peak concentration (Cₘₐₓ) and AUC for Nanoromidepsin by the IV route were 10-fold and 25-fold higher compared to free romidepsin respectively (Table 1). The pharmacokinetic (PK) analyses also indicated that the clearance of romidepsin from the plasma by the IV route of administration was faster compared to the IP route of administration. The peak concentration of romidepsin achieved after IP administration of Nanoromidepsin and romidepsin were 804 nM and 218 nM, respectively. After IV administration, the peak concentration of Nanoromidepsin and free romidepsin were 425 nM and 38 nM, respectively. Based on the in vitro data across the TCL cell lines studied, the IC₅₀ of Nanoromidepsin PDLLA was around 2 to 8 nM. These data suggested that Nanoromidepsin can achieve a concentration 80-400-fold greater than the IC₅₀ of romidepsin with a dose that was only one-half of the MTD dose of Nanoromidepsin (described below in FIG. 5A).

To characterize the biodistribution of Nanoromidepsin, time-dependent tissue and tumor uptake studies were performed. Mice engrafted with the H9-dTomato-luc xenograft were administered Nanoromidepsin co-encapsulated with DiO fluorescent dye by the IV route of administration, in parallel with a control group administered free DiO. Whole body fluorescent images were taken at different time points as illustrated in (FIG. 4B). The organs were harvested from the mice after 72 hours and ex vivo fluorescent images were taken. The whole-body imaging of the mice showed that the fluorescence signal of Nanoromidepsin-DiO treated mice was greater compared to the free DiO treated mice even 5 minutes post-administration and throughout the time course (FIG. 4C). Ex vivo analysis of the organs showed that Nanoromidepsin selectively accumulated in the tumor at 72 hours post-administration, with modest uptake in the liver, which was observed only in free DiO treated mice. (FIG. 4D). The quantification of fluorescent signal in all harvested organs showed a significant (p<0.05) accumulation of Nanoromidepsin in the tumor compared to the free DiO (FIG. 4E). In a similar approach, H9-dTomato-luc engrafted mice were injected with 4 mg/kg romidepsin and Nanoromidepsin. Quantitation of romidepsin by LC-MS/MS in the tumor 24 hours post-administration of Nanoromidepsin and free romidepsin (IV) revealed an intertumoral concentration of romidepsin in the free romidepsin and Nanoromidepsin treated groups of 1.34 and 8.57 ng/mg of protein respectively, suggesting a substantially greater accumulation of drug in animal treated with the Nanoromidepsin. (FIG. 4F).

### Example 5

### Nanoromidepsin Exhibited Superior Tolerability Compared to Free Romidepsin In Vivo

The safety and tolerability of Nanoromidepsin was determined in a single dose toxicity study in female BALB/c mice. Mice were treated with incremental doses of Nanoromidepsin or free romidepsin (IP and IV route) to identify the MTD after single treatment. Changes in body weight and clinical score were assessed as a function of time and dose. While mice in both treatment cohorts experienced weight loss post treatment, the weight returned to pre-treatment levels in most animals after 15 days (FIGS. 5A, 5B, and 10). Mice treated with 8 mg/kg of either free romidepsin or Nanoromidepsin by the IP route met criteria for euthanasia three days post-treatment. At this dose level, about 80% of the mice (4 of 5 mice) treated with romidepsin were found dead three days post-treatment, while only about 40% Nanoromidepsin treated mice (2 of 5 mice) were found dead on the same day. The MTD for both drugs when administered by the IP route was 5 mg/kg. By the IV route of administration, the highest dose studied with each drug was 10 mg/kg. Mice lost approximately 15% body weight within three days after treatment with both 10 mg/kg free romidepsin and Nanoromidepsin, although all mice in both treatment groups recovered after 15 days. Escalation beyond 10 mg/kg was technically not feasible given the volume of the intravenous dose required. Thus, the MTD for both drugs when administered by IV was determined to be 10 mg/kg.

Although the AUC and Cmax of Nanoromidepsin were considerably higher when drug was administered intraperitoneally compared to the intravenous route of administration, a study in H9 xenograft engrafted NSG mice confirmed that the IP route for Nanoromidepsin exhibited unacceptably toxicity (FIG. 11). These findings were consistent with the literature suggesting that many nanoparticles cannot be administered safely by IP owing to the association with peritonitis likely due to the physical features of the particle (Petros & DeSimone, 2010). For these reasons, all in vivo studies hereafter exclusively utilized the intravenous route.

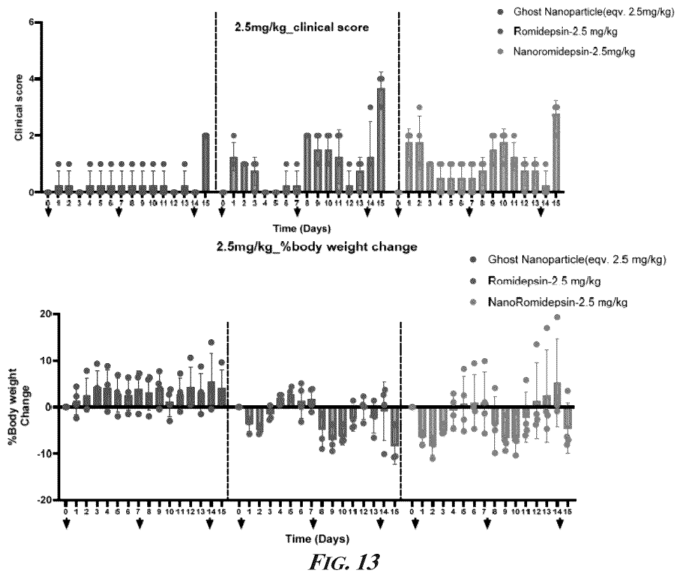

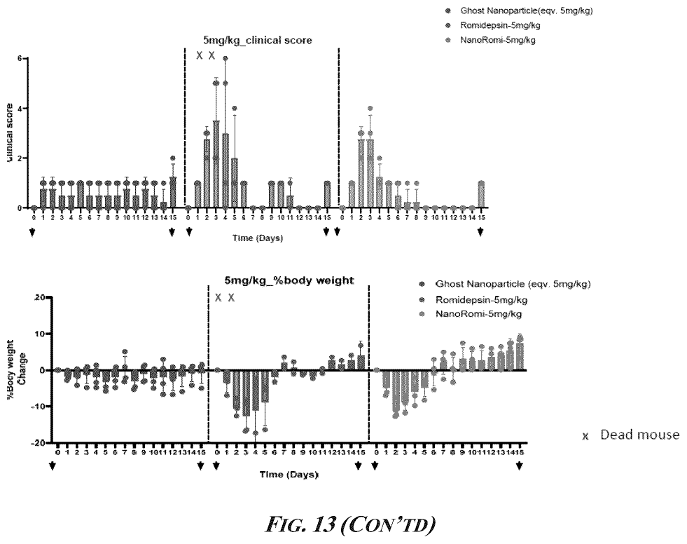

Multiple dose and schedule studies were conducted to assess repeated dosing toxicity in H9-dTomato-luc xenograft-containing mice (Table 2 and FIGS. 12 and 13). The optimum dose and schedule for Nanoromidepsin was identified to be 4 mg/kg weekly for three weeks followed by a one-week rest (FIGS. 5C and 5D). Repeat dosing studies revealed that free romidepsin produced a higher degree of weight loss (>10%) and clinical score (>3) compared to Nanoromidepsin, demonstrating the superior tolerability of the PNP. Free romidepsin at a dose of 8 mg/kg in H9-dTomato-luc xenograft-containing NSG mice demonstrated acute toxicity leading to death of all mice (thus LD₅₀ is significantly less than 8 mg/kg) within four days, while 8 mg/kg Nanoromidepsin was lethal in only 50% of mice, representing the LD₅₀ of Nanoromidepsin. (FIGS. 5E and 5F).

To assess organ-specific toxicity, mice were treated with 4 mg/kg of free romidepsin, ghost PNP or Nanoromidepsin as detailed in FIGS. 5C and 5D (same experiment). Twenty-four hours after the last treatment, liver and tumor were harvested and assessed for pathological findings (FIG. 5G). Liver sections from all mouse cohorts showed normal microarchitecture without any indication of inflammation or necrosis. Tumor sections from the mice treated with the ghost PNP revealed sheet-like infiltrates of large atypical lymphocytes with pleomorphic nuclei, distinct nucleoli and amphophilic cytoplasm, consistent with viable tumor. The romidepsin and Nanoromidepsin treated tumor sections showed varying degrees of treatment related necrosis, with no substantial difference in the histopathology between the two treatment groups. Although there were no signs of drug induced toxicity in the liver sections of either treatment cohort, the LC-MS confirmed that the concentrations of romidepsin in the liver tissue after the administration of free and Nanoromidepsin were 46.68 and 13.18 ng/mg of protein, respectively (p<0.0009; FIG. 5H). The mean plasma concentrations of romidepsin after 1 hour and 24 hours following three consecutive treatments of romidepsin (weekly doses for three weeks) were 51 and 4.9 ng/ml (FIG. 5I). These data suggest a rapid decline in mean plasma concentration 24 hours after the third dose, implying a rapid clearance of free romidepsin from the blood. In contrast, the mean plasma concentrations of romidepsin in the plasma collected at 1 and 24 hours at the same dose of Nanoromidepsin were 120.3 and 40.7 ng/m, (2.3 and 8.3-fold greater than the free drug).

### Example 6

### Nanoromidepsin Showed Superior Activity and a Survival Advantage in Murine Xenograft Models

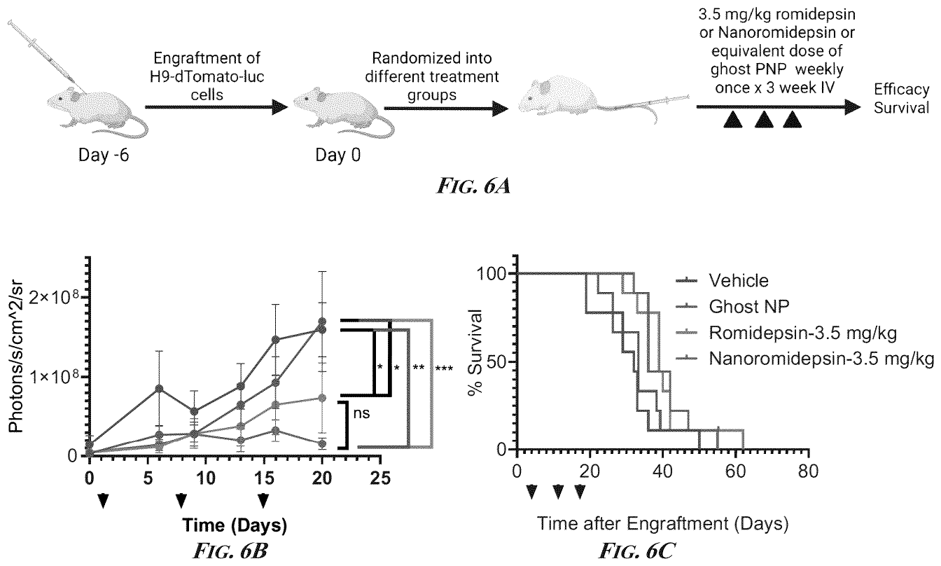

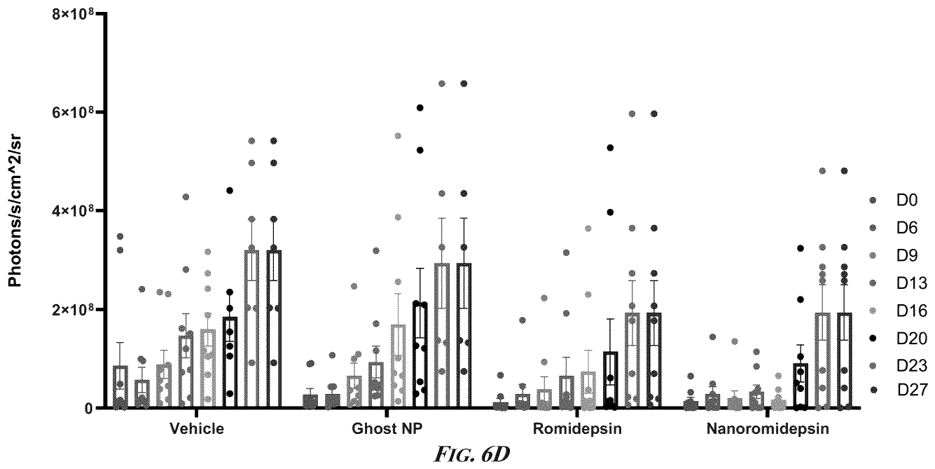

To determine efficacy and differences in overall survival (OS), dTomato-luc expressing H9 cells were engrafted subcutaneously into the right flank of NSG mice. Mice were treated when the tumor luminescence reached 10⁶ bioluminescence intensity (BLI; p/s/cm²/sr), usually 6-7 days after the engraftment. Mice were treated with 3.5 mg/kg weekly for 3 weeks with free romidepsin or Nanoromidepsin (FIG. 6A). After three treatments, the cohort receiving free romidepsin exhibited moderate antitumor activity with tumor growth inhibition of 54% and 57% compared to the vehicle and ghost PNP cohorts respectively (p=0.0315 vs. vehicle; p=0.04 vs. ghost PNP). Treatment with Nanoromidepsin showed inhibition of 90% and 91% compared to the vehicle and ghost PNP cohorts respectively (p=0.0003 vs. vehicle; p=0.0019 vs. ghost PNP), while there was no statistically significant difference in the growth delay observed between romidepsin and Nanoromidepsin (p=0.6665), although Nanoromidepsin showed greater tumor reduction compared to free romidepsin after 3 weeks of treatment (FIG. 6B). The tumor BLI signal was reduced one week after the first treatment which held constant for the next three weeks for both treatment cohorts (FIGS. 6B, 6D, and 6E). Interestingly, the Nanoromidepsin treated cohort showed delayed tumor growth compared to romidepsin until the third week of treatment. However, mice treated with Nanoromidepsin or free romidepsin did not show any statistically significant improvement in survival benefit at this dose or schedule due to cytokinetic failures, which was attributed in part to the fact mice had to receive a lower dose of drug and only one cycle of therapy (FIG. 6C).

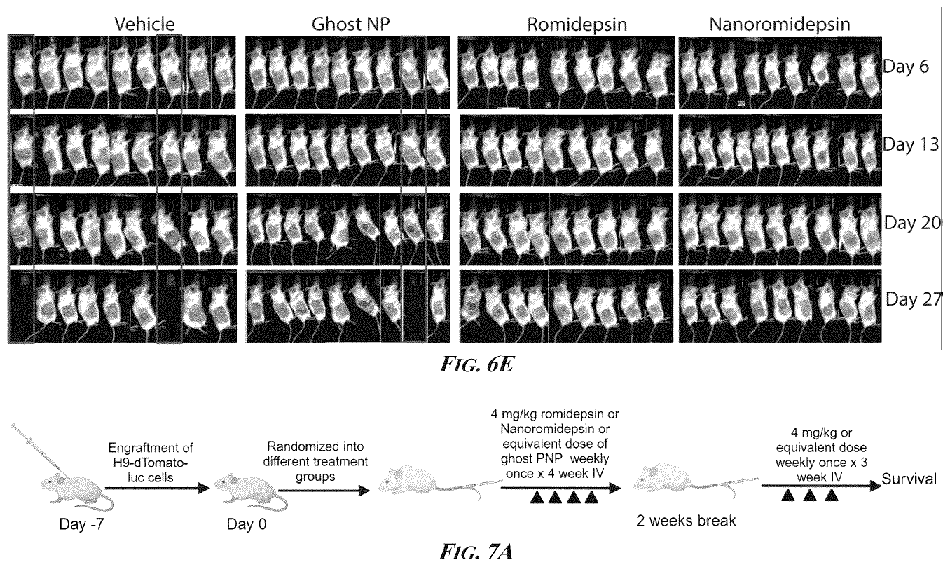

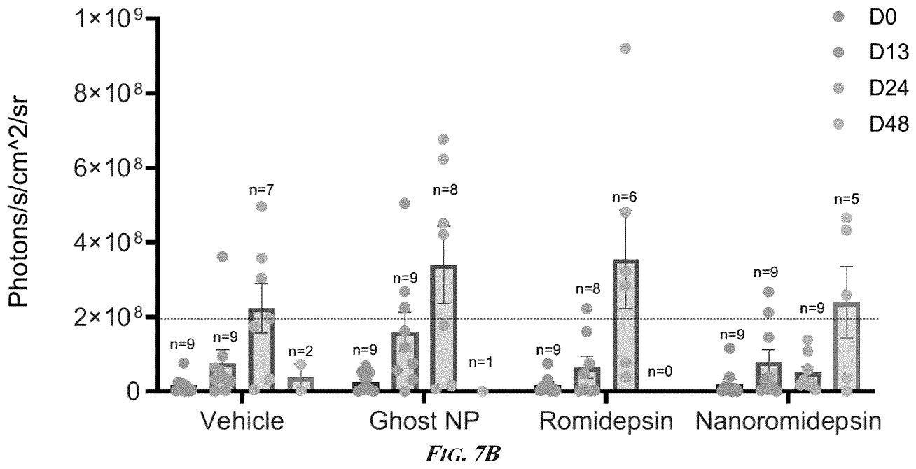

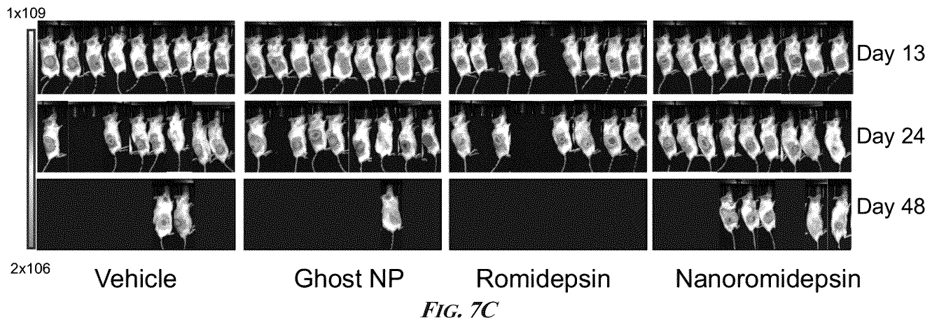

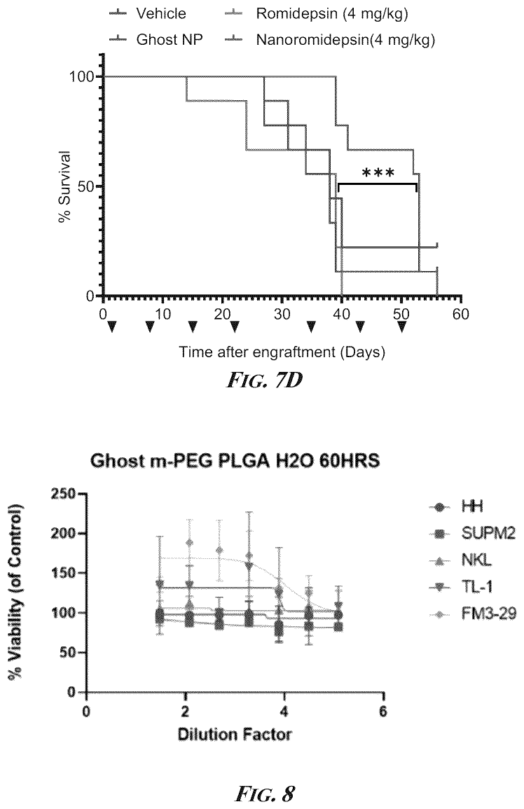

Given the issue of cytokinetic failure, we explored further modifying the dose and schedule, administering both drugs at a higher dose of 4 mg/kg weekly for four consecutive weeks, followed by a two-week break (FIG. 7A). Significant toxicity was noted after one treatment with free romidepsin. As shown in FIG. 7B and C, a consistent increase in the BL1 intensity which is proportional to tumor growth was observed in the PBS, ghost PNP and free romidepsin treated mice cohort until day 24. However, a growth delay was observed in Nanoromidepsin treated mice in the same time frame. Moreover, as shown FIGS. 7C, 33% of mice died after three weeks of treatment with free romidepsin, while treatment with Nanoromidepsin resulted no deaths. Consistent with the bioluminescence imaging data, Nanoromidepsin treatment resulted in a highly statistically significant prolongation in OS compared to the free romidepsin treated mice. The overall survival in the control, ghost PNP and romidepsin treated mice was 38 days (for all three groups). In contrast, the median OS of Nanoromidepsin administered mice was 53 days (p<0.001), which was highly statistically significant in comparison to free romidepsin.

### Discussion of the Examples

The dwindling options to treat patients with relapsed or refractory PTCL have created an urgent need to change the paradigm in how we think about and develop new drugs for challenging orphan diseases. In the U.S., pralatrexate and the HDACi belinostat are the only drugs still approved for patients with R/R PTCL, albeit they have only a dangling accelerated approval. Brentuximab vedotin (Bv), an antibody drug conjugate which targets CD30, has been approved for the treatment of R/R anaplastic large cell lymphoma (ALCL), and in combination with cyclophosphamide, doxorubicin and prednisone (Bv-CHP) for the front-line treatment of CD30-positive PTCL (Horwitz et al., 2019). Most physicians who treat patients with R/R PTCL agree that romidepsin is among one of the more important drugs they prescribe to manage these diseases. Loss of the romidepsin indication in R/R PTCL has put physicians and patients in a challenging position. Couple this with the reality that there are not many new drugs on the horizon, it becomes obvious that new strategies to improve the agents at our disposal, or to create new ones based on promising combinatorial datasets, represent one relatively derisked approach to advance care.

Romidepsin in combination with other epigenetically targeted drugs like the DNMT3 inhibitor 5-azacytidine appears to produce the best overall response rate (ORR) and progression free survival (PFS) data of any ‘drug’ or drug combination to date in this population (O'Connor et al., 2019; Falchi et al., 2021). Nonetheless, these modest clinical and preclinical experiences suggest that combinations with an HDACi, with romidepsin being among the most potent in the class, may represent one relatively straight-forward path to create new treatment platforms for this challenging population. How these combinations get selected may have an impact on the clinical experiences, as those doublets with robust preclinical datasets and a demonstration of drug: drug synergy appear to have generally more favorable outcomes (Jain et al., 2015; Scotto et al., 2021). While romidepsin has been shown to be consistently more potent than other HDACi in the laboratory, in the clinic it produces an ORR of 25%, with a PFS of about 3 to 4 months, and a median duration of response of more than a year (Piekarz et al., 2011; Coiffier et al., 2012). These discordant findings between pre-clinical and clinical experiences remain to be explained. Pharmacokinetic analysis suggests that romidepsin is highly protein-bound (92%-94%), with al-acid-glycoprotein (AAG) being the principal binding protein. Also, an in vitro study showed that romidepsin accumulates in human hepatocytes via an unknown active uptake process (ISTODAX package insert), a finding we validated in the murine model system. After administration of 4 mg/kg free romidepsin and Nanoromidepsin weekly for three weeks in mice, LC-MS based quantification of romidepsin in liver demonstrated greater accumulation of free romidepsin, while mice treated with Nanoromidepsin exhibited substantially less accumulation of romidepsin in the liver (FIG. 5H). One possible explanation for why all HDAC inhibitors produce similar clinical data despite vast differences in potency may relate to the sub-optimized pharmacologic features of the drug, and the fact that the short half-life (estimated to be about 3.8 hours in humans), coupled to a high degree of protein binding and a smaller volume of distribution (Vd) may not maximize romidepsin's effects on transcriptional activation, the primary mechanism of action for the drug. Strategies that optimize the on-target effect, transcription, offer some prospect of improving the effect of any epigenetic based drug. Here, we exploited the unique physicochemical properties of a tailored PNP, including optimal size and surface properties, enhanced volume of distribution (Vd), and augmented tumor bioavailability, in an effort to improve on the fundamental mechanism of action of the epigenetic effect. All of these factors are essential to overcome those factors that might compromise drug activity. The convergence of the drugs' mechanism of action and the features of an PNP offer the prospect of optimizing the anti-tumor effects of the HDACi.

Polymer-based nanoparticles are receiving increasing attention because of their unique properties that dramatically improve many of the liabilities associated with sub-optimized drugs (Kamaly et al., 2012). Amphiphilic block co-polymers offer many advantages over traditional nanoliposomal based technologies, largely because they can accommodate a broader range of drugs with variable solubility features, that is, both lipophilic and hydrophilic molecules. Polymer nanoparticles typically have a size of less than 100 nm, about the size of a virus, which aids in improving the circulation time of the drug and the volume of distribution, allowing for a bioconcentration of drug in tissue, particularly tumor tissue. The bioluminescent in vivo assay which deployed a PNP containing both romidepsin and DIO clearly establishes a predilection for the PNP to bioaccumulate in the tumor microenvironment. Although there is some debate about the precise mechanisms by which polymeric nanoparticles accumulate in the tumor microenvironment, porous and leaking vasculature have been advanced as one possible explanation (Fang et al., 2011). Irrespective of the underlying mechanics, we have clearly shown a penchant for drug to bioaccumulate in tumor. Generally, a variety of polymers have been deployed for the synthesis of PNPs, including polylactides (PLA; Edlund & Albertsson, 2002; Musumeci et al., 2006), poly (lactide co-glycolides) (PLGA; Demento et al., 2009; Danhier et al., 2012), and poly(D,L-Lactic Acid; Deng et al., 2007). These polymers are considered biocompatible, biodegradable and non-toxic, which enhances their elimination, improves their tolerability, and reduces their immunogenicity (Ben-Akiva et al., 2018). Another important component of the PNP is the inclusion of the PEG chain which has been shown to reduce the elimination of the particles via the host immune system and thereby maximizes the circulation time (Cheng et al., 2015). An attractive feature of this platform is that hydrophobic drugs can be readily incorporated and even conjugated to the polymer (Ibrahim et al., 2022; Ji et al., 2022; Ye et al., 2022). Surface conjugation of cell specific targeting ligands to PEG can also be performed. (Haim Zada et al., 2022; Matthew et al., 2022; Pulingam et al., 2022).

Based upon the clear liabilities associated with the present use of romidepsin, and at least the theoretical advantages of leveraging a nanochemistry platform, we initiated a program to synthesize a polymer nanoparticle of romidepsin to overcome some of these well-established liabilities of the drug, while intently focused on trying to improve romidepsin's fundamental epigenetic mechanisms of action to alter gene expression (Fessi et al., 1989; Barichello et al., 1999). PNPs of romidepsin were synthesized using FDA approved GRAS components. We systematically investigated the parameter effects on the physicochemical properties of the nanoparticles particularly the encapsulation efficiency of the drug. We optimized a precise and scalable method of nanoparticle synthesis by using widely accessible GRAS materials, a crucial step for translational therapeutic development (Bhatia et al., 2022).

In general, the physicochemical data suggested that the nanoprecipitation technique optimized the desired parameters of the nanoparticle. DLS data showed the unimodal distribution of the particles, with a PDI of 0.145 with z-average of 46.25 nm in size. We designed these particles to be approximately 50 nm, which has been suggested to be a size feature that facilitates bioconcentration in the tumor microenvironment (Tang et al., 2014). Particles in this size range are thought to extravasate into neovascularized tumor tissue. By optimizing the pharmacologic features of the romidepsin PNP, we demonstrated superior potency in vitro compared to free romidepsin in different subtypes of T-cell lymphoma and LGL leukemia cell lines. In addition, Nanoromidepsin induced apoptosis as evidenced by the increase in histone acetylation followed by cleaved acetylation similar to free romidepsin, aligned with prior evidence.

In patients with T-cell lymphomas, administration of romidepsin at a dose of 14 mg/m2 intravenously over 4-hours on days 1, 8, and 15 of a 28-day cycle results in geometric mean values of maximum plasma concentration (Cₘₐₓ) and area under the plasma concentration-time curve (AUC-∞) of 377 ng/ml and 1549 ng*hr/mL, respectively. Romidepsin administered as a slow IV bolus dose to rats at 0.33 and 0.67 mg/kg (Report 501650; Istodax package insert) achieved a mean AUCo of 10.3 and 18.1 ng*hr/mL, respectively, after a single dose. (Istodax package insert). Recognizing all the cross-species differences, these data suggest that Nanoromidepsin in these murine models approximated or dramatically exceeded those PK parameters established in humans. Following a single intravenous dose of romidepsin and Nanoromidepsin, the Cₘₐₓ was 21.31 and 231 ng/ml respectively. Another major difference was seen in the AUC, which was 231 and 2532 ng/ml for free and Nanoromidepsin respectively. Nanoromidepsin also exhibited a 1.5-fold increase in half-life (t_{1/2}) compared to free romidepsin, indicating prolonged availability in plasma. These PK features of Nanoromidepsin were confirmed in the biodistribution study where Nanoromidepsin was shown to preferentially bioaccumulate in the tumor over other vital organs. This is noteworthy as some conventional polymeric nanoparticles have been shown to accumulate in organs like the spleen, liver, and kidneys, potentially limiting their therapeutic potential (Cheng et al., 2015). These findings are concordant with previous studies indicating that a PNP tailored for the active pharmaceutical ingredient (API) can improve bioavailability and solubility issues, thereby optimizing their mechanism of action (Keck & Müller, 2006; Mugheirbi et al., 2014), a factor that may be especially important for drugs targeting the epigenome.

The improvement in the PK parameters supporting improved drug exposure of course raises concerns about incrementally worse tolerability. As shown in a series of comprehensive single and multiple dose toxicity assays, Nanoromidepsin was found to be substantially safer than free romidepsin, even at the highest doses studied. These data have established a sound basis to identify the MTD, optimal route of administration, and ideal dosing schedule prior to the efficacy studies. The in vivo toxicity assays affirmed that Nanoromidepsin was safer compared to free romidepsin, exhibiting less accumulation in the liver as observed in the biodistribution studies, and as supported by the histopathology and quantitative LC-MS-based quantification of drugs in organs. In a TCL xenograft model, Nanoromidepsin exhibited an LD₅₀ value of 8 mg/kg, while free romidepsin exhibited an LD₅₀ value of 5 mg/kg. Clinically, the most commonly reported hematologic adverse effects of romidepsin include thrombocytopenia, anemia and neutropenia, while the major non-hematologic toxicities include asthenia, infection, and GI disturbance (Foss et al., 2014). The direct comparison of body weight loss and clinical toxicity scores in mice confirmed the superior safety profile of Nanoromidepsin at every dose and schedule studied.

Across all efficacy studies performed, Nanoromidepsin produced substantially superior growth delay, and an overall survival advantage in contrast to free romidepsin. Generally, multiple cycles of drug administration are uncommonly explored in in vivo murine studies, despite it being the standard of care in human patients. This difference makes it difficult to see overall survival advantages in murine models. An overall survival advantage is based on the depth of a complete remission (CR), which in normal clinical practice is usually achieved with multiple cycles of combination therapy. The improved tolerability and efficacy of Nanoromidepsin would suggest that combinations of drugs with Nanoromidepsin will further deepen the CR, likely translating into improved outcomes for patients with PTCL.

In summary, we have pioneered the development of a unique romidepsin polymer nanoparticle, which exhibits superior pharmacokinetic features, tolerability, and efficacy compared to the historically approved drug. The improved effects on transcription may explain the improvement in efficacy and is supported by the biodistribution data confirming marked bioaccumulation in the tumor microenvironment. This study represents the first to interrogate the merits of a polymer nanoparticle platform on the pharmacology and activity of an epigenetically targeted drug in these diseases. Future studies will address the mechanisms that account for the bioaccumulation of the romidepsin PNP in the tumor microenvironment, as well as the differences in gene expression and how this might explain the potent efficacy advantage for Nanoromidepsin. We believe the platform has potentially created an opportunity to reconfigure the traditional treatment paradigms for patients with peripheral T-cell lymphoma, as we now poise this drug for future clinical studies.

### REFERENCES

All references listed in the instant disclosure, including but not limited to all patents, patent applications and publications thereof, scientific journal articles, and database entries (including but not limited to GENBANK® biosequence database entries and including all annotations available therein) are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
Bachy et al. (2022) Romidepsin Plus CHOP Versus CHOP in Patients with Previously Untreated Peripheral T-Cell Lymphoma: Results of the Ro-CHOP Phase III Study (Conducted by LYSA). J Clin Oncol 40:242-251.
Barichello et al. (1999) Encapsulation of hydrophilic and lipophilic drugs in PLGA nanoparticles by the nanoprecipitation method. Drug Dev Ind Pharm 25:471-476.
Ben-Akiva et al. (2018) Polymeric micro-and nanoparticles for immune modulation. Biomater Sci 7:14-30.
Bhatia et al. (2022) Cancer Nanomedicine. Nat Rev Cancer 22:550-556.
Bigouret et al. (2003) Monoclonal T-cell expansions in asymptomatic individuals and in patients with large granular leukemia consist of cytotoxic effector T cells expressing the activating CD94:NKG2C/E and NKD2D killer cell receptors. Blood 101:3198-3204 (2003).
Bourquin et al. (2018) Biodistribution, Clearance, and Long-Term Fate of Clinically Relevant Nanomaterials. Advanced Materials 30:1704307.
Bozzuto & Molinari (2015) Liposomes as nanomedical devices. Int J Nanomedicine 10:975-999.
Bradner et al. (2010) Chemical phylogenetics of histone deacetylases. Nat Chem Biol 6:238-243.
Cheng et al. (2015) A holistic approach to targeting disease with polymeric nanoparticles. Nat Rev Drug Discov 14:239-247.
Coiffier et al. (2012) Results from a pivotal, open-label, phase II study of romidepsin in relapsed or refractory peripheral T-cell lymphoma after prior systemic therapy. J Clin Oncol 30:631-636.
Danhier et al. (2012) PLGA-based nanoparticles: an overview of biomedical applications. J Control Release 161:505-522.
Demento et al. (2009) Inflammasome-activating nanoparticles as modular systems for optimizing vaccine efficacy. Vaccine 27, 3013-3021 (2009).
Deng et al. (2007) FG020326-loaded nanoparticle with PEG and PDLLA improved pharmacodynamics of reversing multidrug resistance in vitro and in vivo. Acta Pharmacol Sin 28:913-920.
Edlund & Albertsson (2002) in Degradable Aliphatic Polyesters. Springer Berlin Heidelberg, Berlin, Heidelberg, pp. 67-112.
Falchi et al. (2021) Combined oral 5-azacytidine and romidepsin are highly effective in patients with PTCL: a multicenter phase 2 study. Blood 137:2161-2170.
Fang et al. (2011) The EPR effect: Unique features of tumor blood vessels for drug delivery, factors involved, and limitations and augmentation of the effect. Adv Drug Deliv Rev 63:136-151.
Fessi et al. (1989) Nanocapsule formation by interfacial polymer deposition following solvent displacement. International Journal of Pharmaceutics 55:1-4.
Foss et al. (2014) Tolerability to romidepsin in patients with relapsed/refractory T-cell lymphoma. Biomark Res 2:16.
Fujiwara et al. (2021) CSF1/CSF1R Signaling Inhibitor Pexidartinib (PLX3397) Reprograms Tumor-Associated Macrophages and Stimulates T-cell Infiltration in the Sarcoma Microenvironment. Mol Cancer Ther 20:1388-1399.
Gagliardi et al. (2021) Biodegradable Polymeric Nanoparticles for Drug Delivery to Solid Tumors. Front Pharmacol 12:601626.
Glozak et al. (2005) Acetylation and deacetylation of non-histone proteins. Gene 363:15-23.
Haim Zada et al. (2022) Peptide loaded polymeric nanoparticles by non-aqueous nanoprecipitation. J Colloid Interface Sci 622:904-913.
Horwitz et al. (2019) Brentuximab vedotin with chemotherapy for CD30-positive peripheral T-cell lymphoma (ECHELON-2): a global, double-blind, randomised, phase 3 trial. Lancet 393:229-240.
Ibrahim et al. (2022) Lyophilized tablets of felodipine-loaded polymeric nanocapsules to enhance aqueous solubility: Formulation and optimization. Journal of Drug Delivery Science and Technology 70:103172.
Jain et al. (2015) Preclinical pharmacologic evaluation of pralatrexate and romidepsin confirms potent synergy of the combination in a murine model of human T-cell lymphoma. Clin Cancer Res 21:2096-2106.
Jayappa et al. (2017) Microenvironmental agonists generate de novo phenotypic resistance to combined ibrutinib plus venetoclax in CLL and MCL. Blood Adv 1:933-946.
Jayappa et al. (2021) Extrinsic interactions in the microenvironment in vivo activate an antiapoptotic multidrug-resistant phenotype in CLL. Blood Adv 5:3497-3510.
Ji et al. (2022) The targeting imaging and treatment capacity of gelsolin-targeted and paclitaxel-loaded PLGA nanoparticles in vitro and in vivo. Front Bioeng Biotechnol 10:933856.
Jiang et al. (2019) Tucidinostat plus exemestane for postmenopausal patients with advanced, hormone receptor-positive breast cancer (ACE): a randomised, double-blind, placebo-controlled, phase 3 trial. Lancet Oncol 20:806-815.
Kamaly et al. (2012) Targeted polymeric therapeutic nanoparticles: design, development and clinical translation. Chem Soc Rev 41:2971-3010.
Keck & Müller (2006) Drug nanocrystals of poorly soluble drugs produced by high pressure homogenisation. Eur J Pharm Biopharm 62:3-16.
Lamy et al. (2017) LGL leukemia: from pathogenesis to treatment. Blood 129:1082-1094.
LeBlanc et al. (2020) Sphingosine kinase-2 is overexpressed in large granular lymphocyte leukaemia and promotes survival through Mcl-1. Br J Haematol 190:405-417.
Lundell et al. (2005) T-cell large granular lymphocyte leukemias have multiple phenotypic abnormalities involving pan-T-cell antigens and receptors for MHC molecules. Am J Clin Pathol 124:937-946.
Matthew et al. (2022) Mixing and flow-induced nanoprecipitation for morphology control of silk fibroin self-assembly. RSC Adv 12:7357-7373.
Mugheirbi et al. (2014) Heat induced evaporative antisolvent nanoprecipitation (HIEAN) of itraconazole. Int J Pharm 471:400-411.
Musumeci et al. (2006) PLA/PLGA nanoparticles for sustained release of docetaxel. Int J Pharm 325:172-179.
O'Connor et al. (2006) Clinical experience with intravenous and oral formulations of the novel histone deacetylase inhibitor suberoylanilide hydroxamic acid in patients with advanced hematologic malignancies. J Clin Oncol 24:166-173.
O'Connor et al. (2015) Belinostat in Patients With Relapsed or Refractory Peripheral T-Cell Lymphoma: Results of the Pivotal Phase II BELIEF (CLN-19) Study. J Clin Oncol 33:2492-2499.
O'Connor et al. (2019) Oral 5-azacytidine and romidepsin exhibit marked activity in patients with PTCL: a multicenter phase 1 study. Blood 134:1395-1405.
O'Malley (2007) T-cell large granular leukemia and related proliferations. Am J Clin Pathol 127:850-859.
Olsen et al. (2007) Phase IIb multicenter trial of vorinostat in patients with persistent, progressive, or treatment refractory cutaneous T-cell lymphoma. J Clin Oncol 25:3109-3115.
PCT International Patent Application Publication No. WO 2023/06463
Petros & DeSimone (2010) Strategies in the design of nanoparticles for therapeutic applications. Nat Rev Drug Discov 9:615-627.
Piekarz et al. (2009) Phase II multi-institutional trial of the histone deacetylase inhibitor romidepsin as monotherapy for patients with cutaneous T-cell lymphoma. J Clin Oncol 27:5410-5417.
Piekarz et al. (2011) Phase 2 trial of romidepsin in patients with peripheral T-cell lymphoma. Blood 117:5827-5834.
Prince et al. (2009) Panobinostat (LBH589): a potent pan-deacetylase inhibitor with promising activity against hematologic and solid tumors. Future Oncol 5:601-612.
Pulingam et al. (2022) Exploring Various Techniques for the Chemical and Biological Synthesis of Polymeric Nanoparticles. Nanomaterials (Basel) 12(3):576.
Ren et al. (2013) Developing an in vitro model of T cell type of large granular lymphocyte leukemia. Leuk Res 37:1737-1743.
Saksouk et al. (2015) Constitutive heterochromatin formation and transcription in mammals. Epigenetics Chromatin 8:3.
Scotto et al. (2021) Targeting the T-Cell Lymphoma Epigenome Induces Cell Death, Cancer Testes Antigens, Immune-Modulatory Signaling Pathways. Molecular Cancer Therapeutics 20:1422-1430.
Seto & Yoshida (2014) Erasers of histone acetylation: the histone deacetylase enzymes. Cold Spring Harb Perspect Biol 6:a018713.
Shi et al. (2015) Results from a multicenter, open-label, pivotal phase II study of chidamide in relapsed or refractory peripheral T-cell lymphoma. Ann Oncol 26:1766-1771.
Shi et al. (2017) Cancer nanomedicine: progress, challenges and opportunities. Nature Reviews Cancer 17:20-37.
Tang et al. (2014) Investigating the optimal size of anticancer nanomedicine. Proc Natl Acad Sci U S A 111:15344-15349.
Tao et al. (2023) Romidepsin-induced durable remission for relapsed nodal peripheral T-cell lymphoma with T follicular helper phenotype after allogeneic hematopoietic cell transplantation. Int J Hematol 118:292-298.
Tenchov et al. (2021) Lipid Nanoparticles-From Liposomes to mRNA Vaccine Delivery, a Landscape of Research Diversity and Advancement. ACS Nano 15:16982-17015.
U.S. Pat. No. 10,092,584.
Whittaker et al. (2010) Final results from a multicenter, international, pivotal study of romidepsin in refractory cutaneous T-cell lymphoma. J Clin Oncol 28:4485-4491.
Yao et al. (2020) Nanoparticle-Based Drug Delivery in Cancer Therapy and Its Role in Overcoming Drug Resistance. Front Mol Biosci 7:193.
Ye et al. (2022) Improved paclitaxel delivery with PEG-b-PLA/zein nanoparticles prepared via flash nanoprecipitation. Int J Biol Macromol 221:486-495.

It will be understood that various details of the presently disclosed subject matter can be changed without departing from the scope of the presently disclosed subject matter. Furthermore, the foregoing description is for the purpose of illustration only, and not for the purpose of limitation.

1. A composition comprising a histone deacetylase inhibitor (HDACi) encapsulated in and/or otherwise associated with a nanoparticle, wherein the nanoparticle further comprises a detectable agent associated therewith, an additional therapeutic agent associated therewith, or a combination thereof.

2. The composition of claim 1, wherein the HDACi is selected from the group consisting of vorinostat, romidepsin, belinostat, and panobinostat, or any combination thereof, optionally wherein the HDACi is romidepsin.

3. The composition of claim 1, wherein the detectable agent comprises a fluorescent moiety, optionally wherein the detectable agent is selected from the group consisting of 3,3′-dioctadecyloxacarbocyanine perchlorate (DiO), rhodamine, indocyanine green, DiD, and PI.

4. The composition of claim 1, comprising one or more polymers and/or one or more surfactants, optionally wherein the one or more surfactants comprise a poloxamer, optionally poloxamer 188.

5. The composition of claim 4, wherein the one or more polymers are selected from the group consisting of poly-d,1-lactic acid (PDLLA), poly(lactic acid; PLA), pegylated derivatives thereof, copolymers thereof, and blends thereof.

6. The composition of claim 4, wherein the polymer comprises a polymer selected from the group consisting of a synthetic polymer; a biodegradable polymer; a biocompatible polymer; an amphiphilic polymer; a diblock co-polymer; and blends thereof.

7. The composition of claim 4, wherein the polymer comprises a hydrophilic PEG chain, optionally methoxy PEG, PEG-carboxylic acid, PEG-hydroxyl, and/or PEG amine as an end cap, and/or a PEG chain length of 0 -10 kilodaltons (kDa).

8. The composition of claim 4, wherein the polymer is a hydrophobic core-forming polymer, optionally a hydrophobic core-forming polymer selected from the group consisting of PDLLA, PLA, and pegylated derivatives thereof.

9. The composition of-any claim 1, wherein the nanoparticle comprises a methyl ether-PEG polylactide-co-glycolide (mPEG-PLGA), a methyl ether-PEG poly-d,1-lactic acid (mPEG-PDLLA), or any combination thereof.

10. The composition of claim 1, wherein one or more parameters selected from a group consisting of mode of phase addition, HDACi/polymer ratio, HDACi/surfactant ratio, solvent/anti-solvent ratio, rate of addition, and combinations thereof are optimized.

11. The composition of claim 10, wherein:
(a) the HDACi/polymer ratio ranges from about 1:10 to about 1:100 W/W, optionally 1:10 to about 1:50 W/W;
(b) the HDACi/surfactant ratio ranges from about 1:0.05 to about 1:0.2 W/W;
(c) the solvent/anti-solvent ratio ranges from about 1:10 to about 1:1, optionally wherein the anti-solvent is selected from the group consisting of water, PBS, or another ionic buffer solution; and/or
(d) the rate of addition ranges from about 10 to about 500 mL/hour, optionally about 10 to about 50 mL/hour.

12. A method for treating a disease, disorder, or condition associated with sensitivity to a histone deacetylase inhibitor (HDACi), the method comprising administering to a subject in need thereof an effective amount of the composition of claim 1.

13. The method of claim 12, wherein the disease, disorder, or condition associated with sensitivity to an HDACi is a tumor and/or a cancer, an inflammatory disease, disorder, or condition; an autimmune disease, disorder, or condition; or any combination thereof.

14. The method of claim 13, wherein the tumor and/or the cancer is selected from the group consisting of cutaneous T cell lymphoma (CTCL), peripheral T cell lymphoma (PTCL), multiple myeloma, large granular lymphocytic leukemia (LGLL), and adult T cell leukemia/lymphoma.

15. A method for inhibiting the growth, proliferation, and/or metastasis of a tumor and/or a cancer associated with sensitivity to a histone deacetylase inhibitor (HDACi), the method comprising administering to a subject in need thereof an effective amount of the composition of claim 1.

16. The method of claim 15, wherein the tumor and/or the cancer is selected from the group consisting of cutaneous T cell lymphoma (CTCL), peripheral T cell lymphoma (PTCL), multiple myeloma, large granular lymphocytic leukemia (LGLL), and adult T cell leukemia/lymphoma.

17. The method of claim 15, further comprising administering to the subject at least one additional treatment, optionally comprising at least one additional therapeutically active agent.

18. The method of claim 17, wherein the at least one additional treatment comprises allogeneic stem cell transplant (allo-SCT).

19. The method of claim 17, wherein the at least one additional therapeutically active agent is a chemotherapeutic agent, optionally wherein the chemotherapeutic agent is selected from the group consisting of acitretin, bexarotene, an interferon, optionally an alpha and/or a gamma interferon, cyclosporine A, methotrexate, romidepsin, vorinostat, cyclophosphamide, doxorubicin, vincristine, prednisone, dexamethasone, etoposide, vincristine, brentuximab vedotin, a histone lysine methyltransferase (HMT) enhancer of zeste homolog 1 (EZH1) inhibitor, a histone lysine methyltransferase (HMT) enhancer of zeste homolog 2 (EZH2) inhibitor, a JAK/STAT pathway inhibitor, optionally ruxolitinib, tofacitinib, baricitinib, fedratinib, upadacitinib, abrocitinib, and/or deucravacitinib; a hypomethylating agent, an immunotherapeutic agent, optionally a monoclonal antibody, further optionally alemtuzumab; ranimustine, vindesine, carboplatin, and combinations thereof.

20. (canceled)

21. The method of claim 19, wherein the combination thereof is selected from the group consisting of cyclophosphamide/doxorubicin/vincristine/prednisone (CHOP); etoposide/vincristine/doxorubicin/cyclophosphamide/prednisone (CHOEP); cyclophosphamide/doxorubicin/prednisone/brentuximab vedotin (CHP-BV); brentuximab vedotin/cyclophosphamide/doxorubicin/prednisone (CHP); and vincristine/cyclophosphamide/doxorubicin/prednisone (VCAP) plus doxorubicin/ranimustine/prednisone (AMP), and vindesine/etoposide/carboplatin/prednisone (VECP; VCAP-AMP-VECP).

22-35. (canceled)
